SEQUENCE LISTING (110 sequences)

SEQ ID NO: 1 - RNA, 23 nt, Homo sapiens
uaauacugccugguaaugaugac

SEQ ID NO: 2 - RNA, 22 nt, Homo sapiens
uaauacugccggguaaugaugg

SEQ ID NO: 3 - RNA, 22 nt, Homo sapiens
uaacacugucugguaacgaugu

SEQ ID NO: 4 - RNA, 22 nt, Homo sapiens
caucuuaccggacagugcugga

SEQ ID NO: 5 - RNA, 22 nt, Homo sapiens
uaauacugucugguaaugccgu

SEQ ID NO: 6 - RNA, 23 nt, Homo sapiens
uaauacugucagguaaagauguc

SEQ ID NO: 7 - RNA, 23 nt, Homo sapiens
uaacacugucugguaacgauguu

SEQ ID NO: 8 - RNA, 22 nt, Homo sapiens
uaacacugucugguaacgaugu

SEQ ID NO: 9 - RNA, 22 nt, Homo sapiens
uaacacugucugguaacgaugu

SEQ ID NO: 10 - RNA, 23 nt, Homo sapiens
uaauacugucagguaaagauguc

SEQ ID NO: 11 - RNA, 22 nt, Homo sapiens
uaauacugucugguaaaaccgu

SEQ ID NO: 12 - RNA, 22 nt, Homo sapiens
uaauacugccugguaaugauga

SEQ ID NO: 13 - RNA, 23 nt, Homo sapiens
uaauacugccugguaaugaugac

SEQ ID NO: 14 - RNA, 22 nt, Homo sapiens
uaacacugucugguaaagaugg

SEQ ID NO: 15 - RNA, 22 nt, Homo sapiens
uaauacugucugguaaugccgu

SEQ ID NO: 16 - RNA, 22 nt, Homo sapiens
uaacacugucugguaaagaugc

SEQ ID NO: 17 - RNA, 22 nt, Homo sapiens
aauacugccggguaaugaugga

SEQ ID NO: 18 - RNA, 23 nt, Homo sapiens
uaauacugccggguaaugaugga

SEQ ID NO: 19 - RNA, 21 nt, Homo sapiens
aacacugucugguaaagaugg

SEQ ID NO: 20 - RNA, 21 nt, Homo sapiens
uaauacugucugguaaugccg

SEQ ID NO: 21 - RNA, 23 nt, Homo sapiens
uaauacugucagguaaagauguc

SEQ ID NO: 22 - RNA, 22 nt, Homo sapiens
uaauacugucugguaaugccgu

SEQ ID NO: 23 - RNA, 23 nt, Homo sapiens
uaauacugucagguaaagauguc

SEQ ID NO: 24 - RNA, 21 nt, Homo sapiens
aacacugucugguaaagaugg

SEQ ID NO: 25 - RNA, 22 nt, Homo sapiens
uaacacugucugguaacgaugc

SEQ ID NO: 26 - RNA, 23 nt, Homo sapiens
uaauacugucagguaaagauguc

SEQ ID NO: 27 - RNA, 22 nt, Homo sapiens
caucuuacuagacagugcugga

SEQ ID NO: 28 - RNA, 22 nt, Homo sapiens
uaacacugucugguaaagaugg

SEQ ID NO: 29 - RNA, 21 nt, Homo sapiens
aacacugucugguaaagaugc

SEQ ID NO: 30 - RNA, 22 nt, Homo sapiens
uaauacugccggguaaugaugg

SEQ ID NO: 31 - RNA, 22 nt, Homo sapiens
uaacacugucugguaacgaugu

SEQ ID NO: 32 - RNA, 22 nt, Homo sapiens
uaacacugucugguaacgaugu

SEQ ID NO: 33 - RNA, 22 nt, Homo sapiens
uaacacugucugguaacgaugu

SEQ ID NO: 34 - RNA, 22 nt, Homo sapiens
uaauacugccugguaaugauga

SEQ ID NO: 35 - RNA, 21 nt, Homo sapiens
aacacugucugguaaagaugg

SEQ ID NO: 36 - RNA, 22 nt, Homo sapiens
uaauacugucugguaaugccgu

SEQ ID NO: 37 - RNA, 22 nt, Homo sapiens
aauacugccggguaaugaugga

SEQ ID NO: 38 - RNA, 22 nt, Homo sapiens
uaacacugucugguaacgaugu

SEQ ID NO: 39 - RNA, 22 nt, Homo sapiens
uaauacugucugguaaugccgu

SEQ ID NO: 40 - RNA, 23 nt, Homo sapiens
uaauacugccugguaaugaugac

SEQ ID NO: 41 - RNA, 22 nt, Homo sapiens
uaauacugucugguaaugccgu

SEQ ID NO: 42 - RNA, 22 nt, Homo sapiens
aauacugccggguaaugaugga

SEQ ID NO: 43 - RNA, 22 nt, Homo sapiens
uaacacugucugguaacgaugu

SEQ ID NO: 44 - RNA, 22 nt, Homo sapiens
uaauacugccugguaaugauga

SEQ ID NO: 45 - RNA, 22 nt, Homo sapiens
uaauacugccugguaaugaugc

SEQ ID NO: 46 - RNA, 22 nt, Homo sapiens
uaacacugucugguaaagaugg

SEQ ID NO: 47 - RNA, 22 nt, Homo sapiens
uaauacugucugguaaugccgu

SEQ ID NO: 48 - RNA, 23 nt, Homo sapiens
uaauacugccugguaaugaugau

SEQ ID NO: 49 - RNA, 22 nt, Homo sapiens
uaauacugccugguaaugauga

SEQ ID NO: 50 - RNA, 21 nt, Homo sapiens
uaauacugccugguaaugaug

SEQ ID NO: 51 - RNA, 22 nt, Homo sapiens
uaauacugucugguaaugccgu

SEQ ID NO: 52 - RNA, 22 nt, Homo sapiens
uaauacugccggguaaugaugg

SEQ ID NO: 53 - RNA, 23 nt, Homo sapiens
uaauacugccugguaaugaugau

SEQ ID NO: 54 - RNA, 22 nt, Homo sapiens
uaacacugucugguaacgaugu

SEQ ID NO: 55 - RNA, 22 nt, Homo sapiens
uaauacugccggguaaugaugg

SEQ ID NO: 56 - RNA, 95 nt, Homo sapiens
ccagcucgggcagccguggccaucuuacugggcagcauuggauggagucaggucucuaauacugccugguaaugaugacggcggagcccugcacg

SEQ ID NO: 57 - RNA, 68 nt, Homo sapiens
cccucgucuuacccagcaguguuugggugcgguugggagucucuaauacugccggguaaugauggagg

SEQ ID NO: 58 - RNA, 90 nt, Homo sapiens
ccgggccccugugagcaucuuaccggacagugcuggauuucccagcuugacucuaacacugucugguaacgauguucaaaggugacccgc

SEQ ID NO: 59 - RNA, 89 nt, Homo sapiens
cuguggcgcuauugccaucuuacugggcagcauuggauuuugucuauguuucuaauacugccugguaaugaugauuauggcgccccaca

SEQ ID NO: 60 - RNA, 95 nt, Homo sapiens
ccaacuugggcagccguggccaucuuacugggcagcauuggauagugucugaucucuaauacugccugguaaugaugacggcggagcccugcacg

SEQ ID NO: 61 - RNA, 87 nt, Homo sapiens
gguccucugugggcaucuuacuagacagugcuggauuucuuggaucuauucuaacacugucugguaacgauguuuaaagggugaacc

SEQ ID NO: 62 - RNA, 87 nt, Homo sapiens
aaggacaucuguucacaucuuaccgggcagcauuagauccuuuagauaccucuaauacugucagguaaagaugucguccguguccuu

SEQ ID NO: 63 - RNA, 68 nt, Homo sapiens; identical to SEQ ID NO: 57

SEQ ID NO: 64 - RNA, 69 nt, Homo sapiens
cccucgucuuacccagcaguguuugggugcugguugggagucucuaauacugccggguaaugauggagg

SEQ ID NO: 65 - RNA, 68 nt, Homo sapiens; identical to SEQ ID NO: 57

SEQ ID NO: 66 - RNA, 68 nt, Homo sapiens
uggaugucuuaccagacaugguuagaucuggaugcaucugucuaauacugucugguaaugccguccau

SEQ ID NO: 67 - RNA, 85 nt, Homo sapiens
gccauuaccaucuuacugggcagcauuggauguucucuguuuuucuaauacugccugguaaugaugauugugguguuucgugcac

SEQ ID NO: 68 - RNA, 85 nt, Homo sapiens
ugccugcugauggaugucuuaccagacaugguuagaucuggauguaucugucuaauacugucugguaaugccguccauccauggc

SEQ ID NO: 69 - RNA, 76 nt, Homo sapiens
ccuguugauaggcgucuuaccagacaugguuagauguaauuauuguugucuaauacugucugguaaugccguccau

SEQ ID NO: 70 - RNA, 83 nt, Homo sapiens
ucucaggauccaucuuacccgacagugcuggauuguacuacuguuguucuaacacugucugguaacgauguuuucugggugac

SEQ ID NO: 71 - RNA, 67 nt, Homo sapiens
ccucgucuuacccagcaguguuugggugcgguugggagucucuaauacugccggguaaugauggagg

SEQ ID NO: 72 - RNA, 86 nt, Homo sapiens
ggcacuuagcagccaucuuaccggacagugcuggacuguauaacuguuuucuaacacugucugguaacgauguuuguugggugacc

SEQ ID NO: 73 - RNA, 85 nt, Homo sapiens
uggaugccuggcuccaucuuacaaggcaguuuuggauguuauaucuucucuaauacugccugguaaugaugcagauggucaucua

SEQ ID NO: 74 - RNA, 95 nt, Homo sapiens
uggccggcccuggguccaucuuccaguacaguguuggauggucuaauugugaagcuccuaacacugucugguaaagauggcccccgggucgguuu

SEQ ID NO: 75 - RNA, 64 nt, Homo sapiens
uggggccaucuuccaguacagugguggauggugaagcuucuaacacugucugguaaagaugccc

SEQ ID NO: 76 - RNA, 82 nt, Homo sapiens
cuuguugauggacgucuuaccagacaugguuagauguaauaacuugugucuaauacugucugguaaugccguccaucacaug

SEQ ID NO: 77 - RNA, 77 nt, Homo sapiens
ccaucuuacgaggcagcauuggauagcaucacuuuuucuaauacugccugguaaugaugaugaucgucgucugcagg

SEQ ID NO: 78 - RNA, 62 nt, Homo sapiens
caucuuaccugacagugcuggauuauacuacuguuguucuaacacugucugguaacgauguu

SEQ ID NO: 79 - RNA, 86 nt, Homo sapiens
gggugucuguucacaucuuaccgggcagcauuagauauguuaucggauauuucuaauacugucagguaaagaugucguccgagccc

SEQ ID NO: 80 - RNA, 69 nt, Homo sapiens; identical to SEQ ID NO: 64

SEQ ID NO: 81 - RNA, 95 nt, Homo sapiens
uggccggcccuggguccaucuuccaguacaguguuggauggucuaauugugaagcuccuaacacugucugguaaagaugcccccgggguggguuc

SEQ ID NO: 82 - RNA, 86 nt, Homo sapiens
gggccucuguggacaucuuaccggacagugcuggauuucucggcucgacucuaacacugucugguaacgauguucaaaggugaccc

SEQ ID NO: 83 - RNA, 95 nt, Homo sapiens
cggccggcccuggguccaucuuccaguacaguguuggauggucuaauugugaagcuccuaacacugucugguaaagauggcucccggguggguuc

SEQ ID NO: 84 - RNA, 85 nt, Homo sapiens
ggaguaucuguucacaucuuaccgggcagcauuagauugaaguugaccuucuaauacugucagguaaagaugucgucaggauucc

SEQ ID NO: 85 - RNA, 68 nt, Homo sapiens
ccaucuuacugggcagcauuggauggugucuguguuucuaauacugccugguaaugaugaugaugggg

SEQ ID NO: 86 - RNA, 83 nt, Homo sapiens
gucucuaggguacaucuuaccugacagugcuuggcuguucacugauguucuaacacugucugguaacgaugcacucuggugac

SEQ ID NO: 87 - RNA, 83 nt, Homo sapiens
cgccggccgaugggcgucuuaccagacaugguuagaccuggcccucugucuaauacugucugguaaaaccguccauccgcugc

SEQ ID NO: 88 - RNA, 68 nt, Homo sapiens
ccccaucuuacccagcaguguuugggugccgcucgggagucucuaauacugccggguaaugauggagg

SEQ ID NO: 89 - RNA, 90 nt, Homo sapiens
cugggccucugugggcaucuuaccggacagugcuggauuucuuggcuugacucuaacacugucugguaacgauguucaaaggugacccac

SEQ ID NO: 90 - RNA, 83 nt, Homo sapiens
ccugcugauggaugucuuaccagacaugguuagaucuggaugcaucugucuaauacugucugguaaugccguccauccacggc

SEQ ID NO: 91 - RNA, 82 nt, Homo sapiens
gguagucgucuccaucuuacgaggcagcauuggauuucauuacuuuuucuaauacugccugguaaugaugaugauugcugcc

SEQ ID NO: 92 - RNA, 59 nt, Homo sapiens
ccaucuuacugggcagcauuggauggugucuggucucuaauacugccugguaaugauga

SEQ ID NO: 93 - RNA, 89 nt, Homo sapiens
ugguccucuauggacaucuuacuagacagugcuggauuuauuuuaucuuuucuaacacugucugguaacgauguuuaaagagugagcca

SEQ ID NO: 94 - RNA, 94 nt, Homo sapiens
ggcugacucugaguccaucuuccagugcaguguuggaugguugaaguacgaagcuccuaacacugucugguaaagauggcccccgggucaguuc

SEQ ID NO: 95 - RNA, 65 nt, Homo sapiens
cgucuuacccagcaguguuugggugcugguugggagucucuaauacugccggguaaugauggagg

SEQ ID NO: 96 - RNA, 86 nt, Homo sapiens
gggccucugugggcaucuuacuagacagugcuggauuuuuggauguacucuaacacugucugguaacgauguuuaaagagggaacc

SEQ ID NO: 97 - RNA, 70 nt, Homo sapiens
gccguggccaucuuacugggcagcauuggauagugucugaucucuaauacugccugguaaugaugacggc

SEQ ID NO: 98 - RNA, 95 nt, Homo sapiens
cggccggcccuggguccaucuuccaguacaguguuggauggucuaauugugaagcuccuaacacugucugguaaagauggcccccggguggguuc

SEQ ID NO: 99 - RNA, 84 nt, Homo sapiens
gccugcugauugcugucuuaccaggcaaaguuagaucuagcuauuucugucuaauacugucugguaaugccgucaaucgcaugg

SEQ ID NO: 100 - RNA, 72 nt, Homo sapiens
ggguccaucuuccagugcaguguuggaugguugaaguaugaagcuccuaacacugucugguaaagauggccc

SEQ ID NO: 101 - RNA, 89 nt, Homo sapiens
cugggccucugugggcaucuuaccggacagugcuggauuucuuggcuugacucuaacacugucugguaacgauguucaaaggugaccca

SEQ ID NO: 102 - RNA, 95 nt, Homo sapiens
uggccggcccuggguucaucuuccaguacaguguuggauggucuaauugugaagcuccuaacacugucugguaaagauggcccccggguggguuc

SEQ ID NO: 103 - RNA, 87 nt, Homo sapiens
aaggacaucuguucacaucuuaccgggcagcauuagauccuuuuuauaacucuaauacugucagguaaagaugucguccguguccuu

SEQ ID NO: 104 - RNA, 77 nt, Homo sapiens
ggugauuaucuccaucuuacgaggcagcauuggauaucaucacuuucucuaauacugccugguaaugaugaugaucg

SEQ ID NO: 105 - RNA, 87 nt, Homo sapiens
ugccuguugaccaaugucuuaccagacaagguuagaucuaguuacucucgucuaauacugucugguaaugccguuggucacauuggc

SEQ ID NO: 106 - RNA, 85 nt, Homo sapiens
agccugcugaugggcgucuuaccagacacgguuagaucuggguucuggugucuaauacugucugguaaugccguucauccauggc

SEQ ID NO: 107 - RNA, 102 nt, Homo sapiens
cacgacggaguaacgguucgcaucuuaccgggcagcauuagaguccugucuauauuuucuaauacugucagguaaagaugucguccgcgcuccacguucguc

SEQ ID NO: 108 - RNA, 87 nt, Homo sapiens
agccuguugauaggcgucuuaccagacaugguuagauguaauuauuguugucuaauacugucugguaaugccguccauuaaauggca

SEQ ID NO: 109 - RNA, 21 nt, Homo sapiens
  misc_feature (21)..(21): n is a, c, g, or u
mawmwurwwdgrwarhgmwgn

SEQ ID NO: 110 - RNA, 21 nt, Homo sapiens
  misc_feature (21)..(21): n is a, c, g, or u
aawacugwcugguaawgaugn

(19) United States
(12) Patent Application Publication
(10) Pub. No.: US 2009/0163435 A1
(43) Pub. Date: Jun. 25, 2009

(54) miR-200 REGULATED GENES AND PATHWAYS AS TARGETS FOR THERAPEUTIC INTERVENTION

(76) Inventors: Andreas G. Bader, Austin, TX (US); Mike W. Byrom, Austin, TX (US); Charles D. Johnson, Austin, TX (US); David Brown, Austin, TX (US)

Correspondence Address: Fullbright & Jaworski L.L.P., 600 Congress Avenue, Suite 2400, Austin, TX, 78701, US

(21) Appl. No.: 12/124,394

(22) Filed: May 21, 2008

Related U.S. Application Data
(60) Provisional application No. 60/939,309, filed on May 21, 2007.

(51) Int. Cl. A61K 31/7088; C12N 5/06; C12Q 1/68

(52) U.S. Cl. 514/44; 435/375; 435/6

(57) ABSTRACT

The present invention concerns methods and compositions for identifying genes or genetic pathways modulated by miR-200, using miR-200 to modulate a gene or gene pathway, using this profile in assessing the condition of a patient and/or treating the patient with an appropriate miRNA.

This application claims Priority to U.S. Provisional Patent Application Ser. No. 60/939,309, filed May 21, 2007 and PCT application No. PCT/US07/78894 filed Sep. 19, 2007, each of which are hereby incorporated by reference in their entirety.

### FIELD OF THE INVENTION

The present invention relates to the fields of molecular biology and medicine. More specifically, the invention relates to methods and compositions for the treatment of diseases or conditions that are affected by miR-200 microRNAs, microRNA expression, and genes and cellular pathways directly and indirectly modulated by such.

### BACKGROUND

In 2001, several groups used a cloning method to isolate and identify a large group of “microRNAs” (miRNAs) from C. elegans, Drosophila, and humans (Lagos-Quintana et al., 2001; Lau et al., 2001; Lee and Ambros, 2001). Several hundreds of miRNAs have been identified in plants and animals—including humans—which do not appear to have endogenous siRNAs. Thus, while similar to siRNAs, miRNAs are distinct.

miRNAs thus far observed have been approximately 21-22 nucleotides in length, and they arise from longer precursors, which are transcribed from non-protein-encoding genes (Carrington and Ambros, 2003). The precursors form structures that fold back on themselves in self-complementary regions; they are then processed by the nuclease Dicer (in animals) or DCL1 (in plants) to generate the short double-stranded miRNA. One of the miRNA strands is incorporated into a complex of proteins and miRNA called the RNA-induced silencing complex (RISC). The miRNA guides the RISC complex to a target mRNA, which is then cleaved or translationally silenced, depending on the degree of sequence complementarity of the miRNA to its target mRNA. Currently, it is believed that perfect or nearly perfect complementarity leads to mRNA degradation, as is most commonly observed in plants. In contrast, imperfect base pairing, as is primarily found in animals, leads to translational silencing. However, recent data suggest additional complexity (Bagga et al., 2005; Lim et al., 2005), and mechanisms of gene silencing by miRNAs remain under intense study.

Recent studies have shown that changes in the expression levels of numerous miRNAs are associated with various cancers (reviewed in Esquela-Kerscher and Slack, 2006; Calin and Croce, 2006). miRNAs have also been implicated in regulating cell growth and cell and tissue differentiation—cellular processes that are associated with the development of cancer.

The inventors previously demonstrated that hsa-miR-200 is involved with the regulation of numerous cell activities that represent intervention points for cancer therapy and for therapy of other diseases and disorders (U.S. patent application Ser. No. 11/141,707 filed May 31, 2005 and Ser. No. 11/273,640 filed Nov. 14, 2005, each of which is incorporated by reference in its entirety). Hsa-miR-200b was found to be overexpressed (at least 50% higher expression) in at least eighty percent of human colon, lung, thyroid, bladder, and breast cancer tumor samples when compared with expression in adjacent normal samples from those organs in the same patients. The inventors also observed that an inhibitor of hsa-miR-200b increased proliferation of normal human breast epithelial cells (MCF12A) by almost 200% when compared with negative controls. Others have observed miR-200b to be over-expressed in cancerous liver cells (Meng et al., 2006).

Bioinformatics analyses suggest that any given miRNA may bind to and alter the expression of up to several hundred different genes. In addition, a single gene may be regulated by several miRNAs. Thus, each miRNA may regulate a complex interaction among genes, gene pathways, and gene networks. Mis-regulation or alteration of these regulatory pathways and networks, involving miRNAs, are likely to contribute to the development of disorders and diseases such as cancer. Although bioinformatics tools are helpful in predicting miRNA binding targets, all have limitations. Because of the imperfect complementarity with their target binding sites, it is difficult to accurately predict the mRNA targets of miRNAs with bioinformatics tools alone. Furthermore, the complicated interactive regulatory networks among miRNAs and target genes make it difficult to accurately predict which genes will actually be mis-regulated in response to a given miRNA.

Correcting gene expression errors by manipulating miRNA expression or by repairing miRNA mis-regulation represent promising methods to repair genetic disorders and cure diseases like cancer. A current, disabling limitation of this approach is that, as mentioned above, the details of the regulatory pathways and networks that are affected by any given miRNA, including miR-200, remain largely unknown. This represents a significant limitation for treatment of cancers in which miR-200 may play a role. A need exists to identify the genes, genetic pathways, and genetic networks that are regulated by or that may regulate hsa-miR-200 expression.

### SUMMARY OF THE INVENTION

The present invention provides additional compositions and methods by identifying genes that are direct targets for miR-200 regulation or that are indirect or downstream targets of regulation following the miR-200-mediated modification of another gene(s) expression. Furthermore, the invention describes gene, disease, and/or physiologic pathways and networks that are influenced by miR-200 and its family members. In certain aspects, compositions of the invention are administered to a subject having, suspected of having, or at risk of developing a metabolic, an immunologic, an infectious, a cardiovascular, a digestive, an endocrine, an ocular, a genitourinary, a blood, a musculoskeletal, a nervous system, a congenital, a respiratory, a skin, or a cancerous disease or condition.

In particular aspects, a subject or patient may be selected for treatment based on expression and/or aberrant expression of one or more miRNA or mRNA. In a further aspect, a subject or patient may be selected for treatment based on aberrations in one or more biologic or physiologic pathway(s), including aberrant expression of one or more gene associated with a pathway, or the aberrant expression of one or more protein encoded by one or more gene associated with a pathway. In still a further aspect, a subject or patient may be selected based on aberrations in miRNA expression, or biologic and/or physiologic pathway(s). A subject may be assessed for sensitivity, resistance, and/or efficacy of a therapy or treatment regime based on the evaluation and/or analysis of miRNA or mRNA expression or lack thereof. A subject may be evaluated for amenability to certain therapy prior to, during, or after administration of one or therapy to a subject or patient. Typically, evaluation or assessment may be done by analysis of miRNA and/or mRNA, as well as combination of other assessment methods that include but are not limited to histology, immunohistochemistry, blood work, etc.

In some embodiments, an infectious disease or condition includes a bacterial, viral, parasite, or fungal infection. Many of these genes and pathways are associated with various cancers and other diseases. Cancerous conditions include, but are not limited to anaplastic large cell lymphoma, breast carcinoma, B-cell lymphoma, cervical carcinoma, chronic lymphoblastic leukemia, colorectal carcinoma, glioma, glioblastoma, gastric carcinoma, hepatocellular carcinoma, leukemia, lung carcinoma, lipoma, multiple myeloma, mesothelioma, non-small cell lung carcinoma, ovarian carcinoma, oesophageal carcinoma, osteosarcoma, pancreatic carcinoma, prostate carcinoma, rhabdomyosarcoma, squamous cell carcinoma of the head and neck, thyroid carcinoma, or testicular tumor, wherein the modulation of one or more gene is sufficient for a therapeutic response. Typically a cancerous condition is an aberrant hyperproliferative condition associated with the uncontrolled growth or inability to undergo cell death, including apoptosis.

The present invention provides methods and compositions for identifying genes that are direct targets for miR-200 regulation or that are downstream targets of regulation following the miR-200-mediated modification of upstream gene expression. Furthermore, the invention describes gene pathways and networks that are influenced by miR-200 expression in biological samples. Many of these genes and pathways are associated with various cancers and other diseases. The altered expression or function of miR-200 in cells would lead to changes in the expression of these key genes and contribute to the development of disease or other conditions. Introducing miR-200 (for diseases where the miRNA is down-regulated) or a miR-200 inhibitor (for diseases where the miRNA is up-regulated) into disease cells or tissues or subjects would result in a therapeutic response. The identities of key genes that are regulated directly or indirectly by miR-200 and the disease with which they are associated are provided herein. In certain aspects a cell may be an epithelial, stromal, or mucosal cell. The cell can be, but is not limited to brain, a glial, a neuronal, a blood, a cervical, a colorectal, an endometrial, a meninges, a lymphoid, a connective tissue, a retinal, an esophageal, a lung, a cardiovascular, a liver, a breast, a bone, a thyroid, a glandular, an adrenal, a pancreatic, a stomach, a intestinal, a kidney, a bladder, a prostate, a uterus, an ovarian, a testicular, a splenic, a skin, a fat, a smooth muscle, a cardiac muscle, or a striated muscle cell. In certain aspects, the cell, tissue, or target may not be defective in miRNA expression yet may still respond therapeutically to expression or over expression of a miRNA. miR-200 could be used as a therapeutic target for any of these diseases. In certain embodiments miR-200 can be used to modulate the activity of miR-200 in a subject, organ, tissue, or cell.

A cell, tissue, or subject may be a cancer cell, a cancerous tissue, harbor cancerous tissue, or be a subject or patient diagnosed or at risk of developing a disease or condition. In certain aspects a cancer cell is a neuronal, glial, lung, liver, brain, breast, bladder, blood, leukemic, lymphoid, colon, endometrial, stomach, skin, ovarian, fat, bone, cervical, esophageal, pancreatic, prostate, kidney, testicular, muscle, or thyroid cell. In still a further aspect cancer includes, but is not limited to anaplastic large cell lymphoma, breast carcinoma, B-cell lymphoma, cervical carcinoma, chronic lymphoblastic leukemia, colorectal carcinoma, glioma, glioblastoma, gastric carcinoma, hepatocellular carcinoma, leukemia, lung carcinoma, lipoma, multiple myeloma, mesothelioma, non-small cell lung carcinoma, ovarian carcinoma, oesophageal carcinoma, osteosarcoma, pancreatic carcinoma, prostate carcinoma, rhabdomyosarcoma, squamous cell carcinoma of the head and neck, thyroid carcinoma, or testicular tumor.

Embodiments of the invention include methods of modulating gene expression, or biologic or physiologic pathways in a cell, a tissue, or a subject comprising administering to the cell, tissue, or subject an amount of an isolated nucleic acid or mimetic thereof comprising a miR-200 nucleic acid, mimetic, or inhibitor in an amount sufficient to modulate the expression of a gene positively or negatively modulated by a miR-200 miRNA. A “miR-200 nucleic acid sequence” or “miR-200 inhibitor” includes the full length precursor of miR-200, or complement thereof or processed (i.e., mature) sequence of miR-200 and related sequences set forth herein, as well as 5, 6, 7, 8, 9, 10, 11, 12, 13, 14, 15, 16, 17, 18, 19, 20, 21, 22, 23, 24, 25, 26, 27, 28, 29 or more nucleotides of a precursor miRNA or its processed sequence, or complement thereof, including all ranges and integers there between. In certain embodiments, the miR-200 nucleic acid sequence or miR-200 inhibitor contains the full-length processed miRNA sequence or complement thereof and is referred to as the “miR-200 full-length processed nucleic acid sequence” or “miR-200 full-length processed inhibitor sequence.” In still further aspects, the miR-200 nucleic acid comprises at least 5, 6, 7, 8, 9, 10, 11, 12, 13, 14, 15, 16, 17, 18, 19, 20, 21, 22, 23, 24, 25, 50 nucleotide (including all ranges and integers there between) segment or complementary segment of a miR-200 that is at least 75, 80, 85, 90, 95, 98, 99 or 100% identical to SEQ ID NO:1 to SEQ ID NO:108. The general term miR-200 includes all members of the miR-200 family that share at least part of a mature miR-200 sequence. Mature miR-200 sequences include hsa-miR-200b UAAUACUGCCUGGUAAUGAUGAC (MIMAT0000318, SEQ ID NO:1); hsa-miR-200c UAAUACUGCCGGGUAAUGAUGG (MIMAT0000617, SEQ ID NO:2); hsa-miR-200a UAACACUGUCUGGUAACGAUGU (MIMAT0000682, SEQ ID NO:3); hsa-miR-200a* CAUCUUACCGGACAGUGCUGGA (MIMAT0001620, SEQ ID NO:4); fru-miR-429 UAAUACUGUCUGGUAAUGCCGU (MIMAT0002979, SEQ ID NO:5); dps-miR-8 UAAUACUGUCAGGUAAAGAUGUC (MIMAT0001210, SEQ ID NO:6); bta-miR-200a UAACACUGUCUGGUAACGAUGUU (MIMAT0003822, SEQ ID NO:7); xtr-miR-200a UAACACUGUCUGGUAACGAUGU (MIMAT0003693, SEQ ID NO:8); mmu-miR-200a UAACACUGUCUGGUAACGAUGU (MIMAT0000519, SEQ ID NO:9); ame-miR-8 UAAUACUGUCAGGUAAAGAUGUC (MIMAT0001490, SEQ ID NO:10); hsa-miR-429 UAAUACUGUCUGGUAAAACCGU (MIMAT0001536, SEQ ID NO: 11); fru-miR-200b UAAUACUGCCUGGUAAUGAUGA (MIMAT0002983, SEQ ID NO:12); mmu-miR-200b UAAUACUGCCUGGUAAUGAUGAC (MIMAT0000233, SEQ ID NO:13); hsa-miR-141 UAACACUGUCUGGUAAAGAUGG (MIMAT0000432, SEQ ID NO:14); cfa-miR-429 UAAUACUGUCUGGUAAUGCCGU (MIMAT0001539, SEQ ID NO:15); mdo-miR-141 UAACACUGUCUG GUAAAGAUGC (MIMAT0004151, SEQ ID NO:16); mml-miR-200c AAUACUGCCGGGUAAUGAUGGA (MIMAT0002195, SEQ ID NO:17); bta-miR-200c UAAUACUGCCGGGUAAUGAUGGA (MIMAT0003823, SEQ ID NO:18); ggo-miR-141 AACACUGUCUGGUAAAGAUGG (MIMAT0002198, SEQ ID NO:19); xla-miR-429 UAAUACUGUCUGGUAAUGCCG (MIMAT0001346, SEQ ID NO:20); bmo-miR-8 UAAUACUGUCAGGUAAAGAUGUC (MIMAT0004193, SEQ ID NO:21); xtr-miR-429 UAAUACUGUCUGGUAAUGCCGU (MIMAT0003703, SEQ ID NO:22); aga-miR-8 UAAUACUGUCAGGUAAAGAUGUC (MIMAT0001525, SEQ ID NO:23); ppy-miR-141 AACACUGUCUGGUAAAGAUGG (MIMAT0002200, SEQ ID NO:24); dre-miR-141 UAACACUGUCUGGUAACGAUGC (MIMAT0001837, SEQ ID NO:25); dme-miR-8 UAAUACUGUCAGGUAAAGAUGUC (MIMAT0000113, SEQ ID NO:26); mdo-miR-200a* CAUCUUACUAGACAGUGCUGGA (MIMAT0004157, SEQ ID NO:27); rno-miR-141 UAACACUGUCUGGUAAAGAUGG (MIMAT0000846, SEQ ID NO:28); ppa-miR-141 AACACUGUCUGGUAAAGAUGC (MIMAT0002201, SEQ ID NO:29); mdo-miR-200c UAAUACUGCCGGGUAAUGAUGG (MIMAT0004150, SEQ ID NO:30); gga-miR-200a UAACACUGUCUGGUAACGAUGU (MIMAT0001171, SEQ ID NO:31); fru-miR-200a UAACACUGUC UGGUAACGAUGU (MIMAT0002981, SEQ ID NO:32); dre-miR-200a UAACACUGUCUGGUAACGAUGU (MIMAT0001861, SEQ ID NO:33); tni-miR-200b UAAUACUGCCUGGUAAUGAUGA (MIMAT0002984, SEQ ID NO:34); mml-miR-141 AACACUGUCUGGUAAAGAUGG (MIMAT0002196, SEQ ID NO:35); mmu-miR-429 UAAUACUGUCUGGUAAUGCCGU (MIMAT0001537, SEQ ID NO:36); ppy-miR-200c AAUACUGCCGGGUAAUGAUGGA (MIMAT0002199, SEQ ID NO:37); mdo-miR-200a UAACACUGUCUGGUAACGAUGU (MIMAT0004158, SEQ ID NO:38); dre-miR-429 UAAUACUGUCUGGUAAUGCCGU (MIMAT0001624, SEQ ID NO:39); rno-miR-200b UAAUACUGCCUGGUAAUGAUGAC (MIMAT0000875, SEQ ID NO:40); gga-miR-429 UAAUACUGUCUGG UAAUGCCGU (MIMAT0003371, SEQ ID NO:41); ggo-miR-200c AAUACUGCCGGGUAAUGAUGGA (MIMAT0002197, SEQ ID NO:42); tni-miR-200a UAACACUGUCUGGUAACGAUGU (MIMAT0002982, SEQ ID NO:43); mdo-miR-200b UAAUACUGCCUGGUAAUGAUGA (MIMAT0004156, SEQ ID NO:44); dre-miR-200c UAAUACUGCCUGGUAAUGAUGC (MIMAT0001863, SEQ ID NO:45); mmu-miR-141 UAACACUGUCUGGUAAAGAUGG (MIMAT0000153, SEQ ID NO:46); rno-miR-429 UAAUACUGUCUGGUAAUGCCGU (MIMAT0001538, SEQ ID NO:47); xtr-miR-200b UAAUACUGCCUGGUAAUGAUGAU (MIMAT0003694, SEQ ID NO:48); dre-miR-200b UAAUACUGCCUGGUAAUGAUGA (MIMAT0001862, SEQ ID NO:49); bta-miR-200b UAAUACUGCCUGGUAAUGAUG (MIMAT0003842, SEQ ID NO:50); tni-miR-429 UAAUACUGUCUGGUAAUGCCGU (MIMAT0002980, SEQ ID NO:51); rno-miR-200c UAAUACUGCCGGGU AAUGAUGG (MIMAT0000873, SEQ ID NO:52); gga-miR-200b UAAUACUGCCUGGUAAUGAUGAU (MIMAT0001172, SEQ ID NO:53); rno-miR-200a UAACACUGUCUGGUAACGAUGU (MIMAT0000874, SEQ ID NO:54); mmu-miR-200c UAAUACUGCCGGGUAAUGAUGG (MIMAT0000657, SEQ ID NO:55) or a complement thereof. In certain aspects, a subset of these miRNAs will be used that include some but not all of the listed miR-200 family members. In one aspect, miR-200 sequences have a consensus sequence of SEQ ID NO:109. In one embodiment only sequences comprising the consensus sequence of AAWACUGWCUGGUAAWGAUGN (SEQ ID NO:110) will be included with all other miRNAs excluded. The term miR-200 includes all members of the mirR-200 family.

A “miR-200 nucleic acid sequence” includes all or a segment of the full length precursor of miR-200 family members. Stem-loop sequences of miR-200 family members include hsa-mir-200b CCAGCUCGGGCAGCCGUGGCCAUCUUACUGGGC AGCAUUGGAUGGAGUCAGGUCUCUAAUACUGCCUGGUAAUGAUGACGGCG GAGCCCUGCACG (MI0000342, SEQ ID NO:56); hsa-mir-200c CCCUCGUCUUACC CAGCAGUGUUUGGGUGCGGUUGGGAGUCUCUAAUACUGCCGGGUAAUGAU GGAGG (MI0000650, SEQ ID NO:57); hsa-mir-200a CCGGGCCCCUGUG AGCAUCUUACCGGACAGUGCUGGAUUUCCCAGCUUGACUCUAACACUGUC UGGUAACGAUGUUCAAAGGUGACCCGC (MI0000737, SEQ ID NO:58); xtr-mir-200b CUGUGGCGCUAUUGCCAUCUUACUGGGCAGCAUUGGAUUUUGU CUAUGUUUCUAAUACUGCCUGGUAAUGAUGAUUAUGGCGCCCCACA (MI0004946, SEQ ID NO:59); rno-mir-200b CCAACUUGGGCAGCCG UGGCCAUCUUACUGGGCAGCAUUGGAUAGUGUCUGAUCUCUAAUACUGCC UGGUAAUGAUGACGGCGGAGCCCUGCACG (MI0000944, SEQ ID NO:60); gga-mir-200a GGUCCUCUGUGGGCAUCUUACUAGACAGUGCUGGAUUUCUUGGA UCUAUUCUAACACUGUCUGGUAACGAUGUUUAAAGGGUGAACC (MI0001249, SEQ ID NO:61); dps-mir-8 AAGGACAUCUGUUCACAUCUU ACCGGGCAGCAUUAGAUCCUUUAGAUACCUCUAAUACUGUCAGGUAAAGA UGUCGUCCGUGUCCUU (MI0001303, SEQ ID NO:62); mml-mir-200c CCCUCGUCUUACCCAGCAGUGUUUGGGUGCGGUUGGGAGUCUCUAAUACU GCCGGGUAAUGAUGGAGG (MI0002484, SEQ ID NO:63); mmu-mir-200c CCCUCGUCUUACCCAGCAGUGUUUGGGUGCUGGUUGGGAGUCUCUAAU ACUGCCGGGUAAUGAUGGAGG (MI0000694, SEQ ID NO:64); ppy-mir-200c CCCUCGUCUUACCCAGCAGUGUUUGGGUGCGGUUGGGAGUCUCUAAUACU GCCGGGUAAUGAUGGAGG (MI0002488, SEQ ID NO:65); xla-mir-429 UGGAUGUCUUACCAGACAUGGUUAGAUCUGGAUGCAUCUGUCUAAUACUG UCUGGUAAUGCCGUCCAU (MI0001451, SEQ ID NO:66); gga-mir-200b GCCAUUACCAUCUUACUGGGCAGCAUUGGAUGUUCUCUGUUUUUCUAAUA CUGCCUGGUAAUGAUGAUUGUGGUGUUUCGUGCAC (MI0001250, SEQ ID NO:67); rno-mir-429 UGCCUGCUGAUGGAUGUCUUACCAGACAUGGUUAGA UCUGGAUGUAUCUGUCUAAUACUGUCUGGUAAUGCCGUCCAUCCAUGGC (MI0001643, SEQ ID NO:68); fru-mir-429 CCUGUUGAUAGGCGUCUUACCAG ACAUGGUUAGAUGUAAUUAUUGUUGUCUAAUACUGUCUGGUAAUGCCGUC CAU (MI0003301, SEQ ID NO:69); fru-mir-200a UCUCAGGAUCCAUCUUACCCGA CAGUGCUGGAUUGUACUACUGUUGUUCUAACACUGUCUGGUAACGAUGUU UUCUGGGUGAC (MI0003303, SEQ ID NO:70); ggo-mir-200c CCUCGUCUUAC CCAGCAGUGUUUGGGUGCGGUUGGGAGUCUCUAAUACUGCCGGGUAAUGA UGGAGG (MI0002486, SEQ ID NO:71); dre-mir-200a GGCACUUAGCAGCCAUCUUACCGGACAGUGCUGGACUGUAUAACUGUUUU CUAACACUGUCUGGUAACGAUGUUUGUUGGGUGACC (MI0002037, SEQ ID NO:72); dre-mir-200c UGGAUGCCUGGCUCCAUCUUACAAGGCAGUUUUGGAU GUUAUAUCUUCUCUAAUACUGCCUGGUAAUGAUGCAGAUGGUCAUCUA (MI0002039, SEQ ID NO:73); mml-mir-141 UGGCCGGCCCUG GGUCCAUCUUCCAGUACAGUGUUGGAUGGUCUAAUUGUGAAGCUCCUAAC ACUGUCUGGUAAAGAUGGCCCCCGGGUCGGUUU (MI0002485, SEQ ID NO:74); mdo-mir-141 UGGGGCCAUCUUCCAGUACAGUGGUGGAUGGUGAAG CUUCUAACACUGUCUGGUAAAGAUGCCC (MI0005340, SEQ ID NO:75); dre-mir-429 CUUGUUGAUGGACGUCUUACCAGACAUGGUUAGAUGUAAUAAC UUGUGUCUAAUACUGUCUGGUAAUGCCGUCCAUCACAUG (MI0001720, SEQ ID NO:76); tni-mir-200b CCAUCUUACGAGGCAGCAUUGGAUAGCAUCAC UUUUUCUAAUACUGCCUGGUAAUGAUGAUGAUCGUCGUCUGCAGG (MI0003306, SEQ ID NO:77); tni-mir-200a CAUCUUACCUGACAGUGCUGGAUUA UACUACUGUUGUUCUAACACUGUCUGGUAACGAUGUU (MI0003304, SEQ ID NO:78); aga-mir-8 GGGUGUCUGUUCACAUCUUACCGGGCAGCAUUA GAUAUGUUAUCGGAUAUUUCUAAUACUGUCAGGUAAAGAUGUCGUCCGAG CCC (MI0001630, SEQ ID NO:79); rno-mir-200c CCCUCGUCUUACC CAGCAGUGUUUGGGUGCUGGUUGGGAGUCUCUAAUACUGCCGGGUAAUGA UGGAGG (MI0000942, SEQ ID NO:80); ppa-mir-141 UGGCCGGCCCUGGGUCCA UCUUCCAGUACAGUGUUGGAUGGUCUAAUUGUGAAGCUCCUAACACUGUC UGGUAAAGAUGCCCCCGGGGUGGGUUC (MI0002490, SEQ ID NO:81); bta-mir-200a GGGCCUCUGUGGACAUCUUACCGGACAGUGCUGGAUUUCUCGG CUCGACUCUAACACUGUCUGGUAACGAUGUUCAAAGGUGACCC (MI0005037, SEQ ID NO:82); hsa-mir-141 CGGCCGGCCCUGGGUCCAUCU UCCAGUACAGUGUUGGAUGGUCUAAUUGUGAAGCUCCUAACACUGUCUGG UAAAGAUGGCUCCCGGGUGGGUUC (MI0000457, SEQ ID NO:83); ame-mir-8 GGAGUAUCUGUUCACAUCUUACCGGGCAGCAUUAGAUUGAAGUUGACCUU CUAAUACUGUCAGGUAAAGAUGUCGUCAGGAUUCC (MI0001595, SEQ ID NO:84); mdo-mir-200b CCAUCUUACUGGGCAGCAUUGGAUGGUGUCU GUGUUUCUAAUACUGCCUGGUAAUGAUGAUGAUGGGG (MI0005345, SEQ ID NO:85); dre-mir-141 GUCUCUAGGGUACAUCUUACCUGACAGUGCUUGGC UGUUCACUGAUGUUCUAACACUGUCUGGUAACGAUGCACUCUGGUGAC (MI0002004, SEQ ID NO:86); hsa-mir-429 CGCCGGCCGA UGGGCGUCUUACCAGACAUGGUUAGACCUGGCCCUCUGUCUAAUACUGUC UGGUAAAACCGUCCAUCCGCUGC (MI0001641, SEQ ID NO:87); mdo-mir-200c CCCCAUCUUACCCAGCAGUGUUUGGGUGCCGCUCGGGAGUCUCUAAUACUG CCGGGUAAUGAUGGAGG (MI0005339, SEQ ID NO:88); mmu-mir-200a CUGGGCCUCUGUGGGCAUCUUACCGGACAGUGCUGGAUUUCUUGGCUUGA CUCUAACACUGUCUGGUAACGAUGUUCAAAGGUGACCCAC (MI0000554, SEQ ID NO:89); mmu-mir-429 CCUGCUGAUGGAUGUCUUACCAGACAUGGUUA GAUCUGGAUGCAUCUGUCUAAUACUGUCUGGUAAUGCCGUCCAUCCACGG C (MI0001642, SEQ ID NO:90); dre-mir-200b GGUAGUCGUCUCCAUCUUACGAGGCAGCAUUGGAUUUCAUUACUUUUUCU AAUACUGCCUGGUAAUGAUGAUGAUUGCUGCC (MI0002038, SEQ ID NO:91); bta-mir-200b CCAUCUUACUGGGCAGCAUUGGAUGGUGUCUGGUCUCUAAUA CUGCCUGGUAAUGAUGA (MI0005055, SEQ ID NO:92); xtr-mir-200a UGGUCCUCUAUGGACAUCUUACUAGACAGUGCUGGAUUUAUUUUAUCUUU UCUAACACUGUCUGGUAACGAUGUUUAAAGAGUGAGCCA (MI0004945, SEQ ID NO:93); rno-mir-141 GGCUGACUCUGAGUCCAUCUUCCAGUGCAGUGU UGGAUGGUUGAAGUACGAAGCUCCUAACACUGUCUGGUAAAGAUGGCCCC CGGGUCAGUUC (MI0000914, SEQ ID NO:94); bta-mir-200c CGUCUUACCCAGCAGUGUUUGGGUGCUGGUUGGGAGUCUCUAAUACUGCC GGGUAAUGAUGGAGG (MI0005038, SEQ ID NO:95); mdo-mir-200a GGGCCUCUGUGGGCAUCUUACUAGACAGUGCUGGAUUUUUGGAUGUACUC UAACACUGUCUGGUAACGAUGUUUAAAGAGGGAACC (MI0005346, SEQ ID NO:96); mmu-mir-200b GCCGUGGCCAUCUUACUGGGCAGCAUUGGAU AGUGUCUGAUCUCUAAUACUGCCUGGUAAUGAUGACGGC (MI0000243, SEQ ID NO:97); ggo-mir-141 CGGCCGGCCCUGGGUCCAUCUUCCAGUACAGUGU UGGAUGGUCUAAUUGUGAAGCUCCUAACACUGUCUGGUAAAGAUGGCCCC CGGGUGGGUUC (MI0002487, SEQ ID NO:98); gga-mir-429 GCCUGCUGAUUGCUGUCUUACCAGGCAAAGUUAGAUCUAGCUAUUUCUGU CUAAUACUGUCUGGUAAUGCCGUCAAUCGCAUGG (MI0003714, SEQ ID NO:99); mmu-mir-141 GGGUCCAUCUUCCAGUGCAGUGUUGGAUGGUU GAAGUAUGAAGCUCCUAACACUGUCUGGUAAAGAUGGCCC (MI0000166, SEQ ID NO:100); rno-mir-200a CUGGGCCUCUGUGGGCAU CUUACCGGACAGUGCUGGAUUUCUUGGCUUGACUCUAACACUGUCUGGUA ACGAUGUUCAAAGGUGACCCA (MI0000943, SEQ ID NO:101); ppy-mir-141 UGGCCGGCCCUGGGUUCAUCUUCCAGUACAGUGUUGGAUGGUCUAAUUGU GAAGCUCCUAACACUGUCUGGUAAAGAUGGCCCCCGGGUGGGUUC (MI0002489, SEQ ID NO:102); dme-mir-8 AAGGACAUCUGUUCACAUCUUAC CGGGCAGCAUUAGAUCCUUUUUAUAACUCUAAUACUGUCAGGUAAAGAUG UCGUCCGUGUCCUU (MI0000128, SEQ ID NO:103); fru-mir-200b GGUGAUUAUCUCCAUCUUACGAGGCAGCAUUGGAUAUCAUCACUUUCUCU AAUACUGCCUGGUAAUGAUGAUGAUCG (MI0003305, SEQ ID NO:104); xtr-mir-429 UGCCUGUUGACCAAUGUCUUACCAGACAAGGUUAGAUCUAGUUA CUCUCGUCUAAUACUGUCUGGUAAUGCCGUUGGUCACAUUGGC (MI0004956, SEQ ID NO:105); cfa-mir-429 AGCCUGCUGAUGGGCGUCUUACCAG ACACGGUUAGAUCUGGGUUCUGGUGUCUAAUACUGUCUGGUAAUGCCGUU CAUCCAUGGC (MI0001644, SEQ ID NO:106); bmo-mir-8 CACGACGGAGUAACGGUUCGCAUCUUACCGGGCAGCAUUAGAGUCCUGUC UAUAUUUUCUAAUACUGUCAGGUAAAGAUGUCGUCCGCGCUCCACGUUCG UC (MI0004971, SEQ ID NO:107); and tni-mir-429 AGCC UGUUGAUAGGCGUCUUACCAGACAUGGUUAGAUGUAAUUAUUGUUGUCUA AUACUGUCUGGUAAUGCCGUCCAUUAAAUGGCA (MI0003302, SEQ ID NO:108).

In certain aspects, a nucleic acid miR-200 nucleic acid, or a segment or a mimetic thereof, will comprise 5, 6, 7, 8, 9, 10, 11, 12, 13, 14, 15, 16, 17, 18, 19, 20, 21, 22, 23, 24, 25, 26, 27, 28, 29 or more nucleotides of the precursor miRNA or its processed sequence, including all ranges and integers there between. In certain embodiments, the miR-200 nucleic acid sequence contains the full-length processed miRNA sequence and is referred to as the “miR-200 full-length processed nucleic acid sequence.” In still further aspects, a miR-200 comprises at least one 5, 6, 7, 8, 9, 10, 11, 12, 13, 14, 15, 16, 17, 18, 19, 20, 21, 22, 23, 24, 25, 50 nucleotide (including all ranges and integers there between) segment of miR-200 that is at least 75, 80, 85, 90, 95, 98, 99 or 100% identical to SEQ ID NOs provided herein.

In specific embodiments, a miR-200 or miR-200 inhibitor containing nucleic acid is hsa-miR-200 or hsa-miR-200 inhibitor, or a variation thereof. miR-200 can be hsa-miR-200a or hsa-miR-200b or hsa-miR-200c or hsa-miR-200a*. In a further aspect, a miR-200 nucleic acid or miR-200 inhibitor can be administered with 1, 2, 3, 4, 5, 6, 7, 8, 9, 10 or more miRNAs or miRNA inhibitors. miRNAs or their complements can be administer concurrently, in sequence or in an ordered progression. In certain aspects, a miR-200 or miR-200 inhibitor can be administered in combination with one or more of let-7, miR-15, miR-16, miR-20, miR-21, miR-26a, miR-34a, miR-126, miR-143, miR-147, miR-188, miR-215, miR-216, miR-292-3p, and/or miR-331. All or combinations of miRNAs or inhibitors thereof may be administered in a single formulation. Administration may be before, during or after a second therapy.

miR-200 nucleic acids or complement thereof may also include various heterologous nucleic acid sequence, i.e., those sequences not typically found operatively coupled with miR-200 in nature, such as promoters, enhancers, and the like. The miR-200 nucleic acid is a recombinant nucleic acid, and can be a ribonucleic acid or a deoxyribonucleic acid. The recombinant nucleic acid may comprise a miR-200 or miR-200 inhibitor expression cassette, i.e., a nucleic acid segment that expresses a nucleic acid when introduce into an environment containing components for nucleic acid synthesis. In a further aspect, the expression cassette is comprised in a viral vector, or plasmid DNA vector or other therapeutic nucleic acid vector or delivery vehicle, including liposomes and the like. In a particular aspect, the miR-200 nucleic acid is a synthetic nucleic acid. Moreover, nucleic acids of the invention may be fully or partially synthetic. In certain aspects, viral vectors can be administered at 1×10², 1×10³, 1×10⁴ 1×10⁵, 1×10⁶, 1×10⁷, 1×10⁸, 1×10⁹, 1×10¹⁰, 1×10¹¹, 1×10¹², 1×10¹³, 1×10¹⁴ pfu or viral particle (vp).

In a particular aspect, the miR-200 nucleic acid or miR-200 inhibitor is a synthetic nucleic acid. Moreover, nucleic acids of the invention may be fully or partially synthetic. In still further aspects, a nucleic acid of the invention or a DNA encoding such a nucleic acid of the invention can be administered at 0.001, 0.01, 0.1, 1, 10, 20, 30, 40, 50, 100, 200, 400, 600, 800, 1000, 2000, to 4000 μg or mg, including all values and ranges there between. In yet a further aspect, nucleic acids of the invention, including synthetic nucleic acid, can be administered at 0.001, 0.01, 0.1, 1, 10, 20, 30, 40, 50, 100, to 200 μg or mg per kilogram (kg) of body weight. Each of the amounts described herein may be administered over a period of time, including 0.5, 1, 2, 3, 4, 5, 6, 7, 8, 9, 10, minutes, hours, days, weeks, months or years, including all values and ranges there between.

In certain embodiments, administration of the composition(s) can be enteral or parenteral. In certain aspects, enteral administration is oral. In further aspects, parenteral administration is intralesional, intravascular, intracranial, intrapleural, intratumoral, intraperitoneal, intramuscular, intralymphatic, intraglandular, subcutaneous, topical, intrabronchial, intratracheal, intranasal, inhaled, or instilled. Compositions of the invention may be administered regionally or locally and not necessarily directly into a lesion.

In certain aspects, the gene or genes modulated comprises 1, 2, 3, 4, 5, 6, 7, 8, 9, 10, 11, 12, 13, 14, 15, 20, 25, 30, 35, 40, 45, 50, 100, 150, 200 or more genes or combinations of genes identified in Tables 1, 3, 4, and/or 5. In still further aspects, the gene or genes modulated may exclude 1, 2, 3, 4, 5, 6, 7, 8, 9, 10, 11, 12, 13, 14, 15, 20, 25, 30, 35, 40, 45, 50, 100, 150, 175 or more genes or combinations of genes identified in Tables 1, 3, 4, and/or 5. Modulation includes modulating transcription, mRNA levels, mRNA translation, and/or protein levels in a cell, tissue, or organ. In certain aspects the expression of a gene or level of a gene product, such as mRNA or encoded protein, is down-regulated or up-regulated. In a particular aspect the gene modulated comprises or is selected from (and may even exclude) 1, 2, 3, 4, 5, 6, 7, 8, 9, 10, 11, 12, 13, 14, 15, 16, 17, 18, 19, 20, 21, 22, 23, 24, 25, 26, 27, 28, or all of the genes identified in Tables 1, 3, 4, and/or 5, or any combinations thereof. In certain embodiments a gene modulated or selected to be modulated is from Table 1. In further embodiments a gene modulated or selected to be modulated is from Table 3. In still further embodiments a gene modulated or selected to be modulated is from Table 4. In yet further embodiments a gene modulated or selected to be modulated is from Table 5. Embodiments of the invention may also include obtaining or assessing a gene expression profile or miRNA profile of a target cell prior to selecting the mode of treatment, e.g., administration of a miR-200 nucleic acid, inhibitor of miR-200, or mimetics thereof. The database content related to all nucleic acids and genes designated by an accession number or a database submission are incorporated herein by reference as of the filing date of this application. In certain aspects of the invention one or more miRNA or miRNA inhibitor may modulate a single gene. In a further aspect, one or more genes in one or more genetic, cellular, or physiologic pathways can be modulated by one or more miRNAs or complements thereof, including miR-200 nucleic acids and miR-200 inhibitors in combination with other miRNAs.

miR-200 nucleic acids may also include various heterologous nucleic acid sequence, i.e., those sequences not typically found operatively coupled with miR-200 in nature, such as promoters, enhancers, and the like. The miR-200 nucleic acid is a recombinant nucleic acid, and can be a ribonucleic acid or a deoxyribonucleic acid. The recombinant nucleic acid may comprise a miR-200 expression cassette. In a further aspect, the expression cassette is comprised in a viral, or plasmid DNA vector or other therapeutic nucleic acid vector or delivery vehicle, including liposomes and the like. In a particular aspect, the miR-200 nucleic acid is a synthetic nucleic acid. Moreover, nucleic acids of the invention may be fully or partially synthetic.

A further embodiment of the invention is directed to methods of modulating a cellular pathway comprising administering to the cell an amount of an isolated nucleic acid comprising a miR-200 nucleic acid sequence in an amount sufficient to modulate the expression, function, status, or state of a cellular pathway, in particular those pathways described in Table 2 or the pathways known to include one or more genes from Table 1, 3, 4, and/or 5. Modulation of a cellular pathway includes, but is not limited to modulating the expression of one or more gene. Modulation of a gene can include inhibiting the function of an endogenous miRNA or providing a functional miRNA to a cell, tissue, or subject. Modulation refers to the expression levels or activities of a gene or its related gene product or protein, e.g., the mRNA levels may be modulated or the translation of an mRNA may be modulated, etc. Modulation may increase or up regulate a gene or gene product or it may decrease or down regulate a gene or gene product.

Still a further embodiment includes methods of treating a patient with a pathological condition comprising one or more of step (a) administering to the patient an amount of an isolated nucleic acid comprising a miR-200 nucleic acid sequence in an amount sufficient to modulate the expression of a cellular pathway; and (b) administering a second therapy, wherein the modulation of the cellular pathway sensitizes the patient to the second therapy. A cellular pathway may include, but is not limited to one or more pathway described in Table 2 below or a pathway that is know to include one or more genes of Tables 1, 3, 4, and/or 5. A second therapy can include administration of a second miRNA or therapeutic nucleic acid, or may include various standard therapies, such as chemotherapy, radiation therapy, drug therapy, immunotherapy, and the like. Embodiments of the invention may also include the determination or assessment of a gene expression profile for the selection of an appropriate therapy.

Embodiments of the invention include methods of treating a subject with a pathological condition comprising one or more of the steps of (a) determining an expression profile of one or more genes selected from Table 1, 3, 4, and/or 5; (b) assessing the sensitivity of the subject to therapy based on the expression profile; (c) selecting a therapy based on the assessed sensitivity; and (d) treating the subject using selected therapy. Typically, the pathological condition will have as a component, indicator, or result the mis-regulation of one or more gene of Table 1, 3, 4, and/or 5.

Further embodiments include the identification and assessment of an expression profile indicative of miR-200 status in a cell or tissue comprising expression assessment of one or more gene from Table 1, 3, 4, and/or 5, or any combination thereof.

The term “miRNA” is used according to its ordinary and plain meaning and refers to a microRNA molecule found in eukaryotes that is involved in RNA-based gene regulation. See, e.g., Carrington et al., 2003, which is hereby incorporated by reference. The term can be used to refer to the single-stranded RNA molecule processed from a precursor or in certain instances the precursor itself.

In some embodiments, it may be useful to know whether a cell expresses a particular miRNA endogenously or whether such expression is affected under particular conditions or when it is in a particular disease state. Thus, in some embodiments of the invention, methods include assaying a cell or a sample containing a cell for the presence of one or more marker gene or mRNA or other analyte indicative of the expression level of a gene of interest. Consequently, in some embodiments, methods include a step of generating an RNA profile for a sample. The term “RNA profile” or “gene expression profile” refers to a set of data regarding the expression pattern for one or more gene or genetic marker in the sample (e.g., a plurality of nucleic acid probes that identify one or more markers from Tables 1, 3, 4, and/or 5); it is contemplated that the nucleic acid profile can be obtained using a set of RNAs, using for example nucleic acid amplification or hybridization techniques well know to one of ordinary skill in the art. The difference in the expression profile in the sample from the patient and a reference expression profile, such as an expression profile from a normal or non-pathologic sample, is indicative of a pathologic, disease, or cancerous condition. A nucleic acid or probe set comprising or identifying a segment of a corresponding mRNA can include all or part of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 100, 200, 500, or more nucleotides, including any integer or range derivable there between, of a gene, genetic marker, nucleic acid, mRNA or a probe representative thereof that is listed in Tables 1, 3, 4, and/or 5 or identified by the methods described herein.

Certain embodiments of the invention are directed to compositions and methods for assessing, prognosing, or treating a pathological condition in a patient comprising measuring or determining an expression profile of one or more marker(s) in a sample from the patient, wherein a difference in the expression profile in the sample from the patient and an expression profile of a normal sample or reference expression profile is indicative of pathological condition and particularly cancer (e.g., In certain aspects of the invention, the cellular pathway, gene, or genetic marker is or is representative of one or more pathway or marker described in Table 1, 3, 4, and/or 5, including any combination thereof.

Aspects of the invention include diagnosing, assessing, or treating a pathologic condition or preventing a pathologic condition from manifesting. For example, the methods can be used to screen for a pathological condition; assess prognosis of a pathological condition; stage a pathological condition; assess response of a pathological condition to therapy; or to modulate the expression of a gene, genes, or related pathway as a first therapy or to render a subject sensitive or more responsive to a second therapy. In particular aspects, assessing the pathological condition of the patient can be assessing prognosis of the patient. Prognosis may include, but is not limited to an estimation of the time or expected time of survival, assessment of response to a therapy, and the like. In certain aspects, the altered expression of one or more gene or marker is prognostic for a patient having a pathologic condition, wherein the marker is one or more of Table 1, 3, 4, and/or 5, including any combination thereof.

TABLE 1
Genes with increased (positive values) or decreased (negative values)
expression following transfection of human cancer cells with pre-miR hsa-miR-200c

| Gene Symbol | RefSeq Transcript ID (Pruitt et al., 2005) | Δ log₂ |
|---|---|---|
| ABCC1 | NM_004996 /// NM_019862 /// NM_019898 | −0.706813556 |
| | /// NM_019899 /// NM_019900 /// NM_019901 | |
| ACSM3 | NM_005622 /// NM_202000 | −0.716948957 |
| AGR2 | NM_006408 | 1.226546732 |
| AKAP12 | NM_005100 /// NM_144497 | 0.829729605 |
| AP1S2 | NM_003916 | −0.926048874 |
| AREG | NM_001657 | 1.22064281 |
| ARF7 | NM_025047 | 1.710200384 |
| ARG2 | NM_001172 | 0.717825311 |
| ARHGAP8 /// | NM_001017526 /// NM_181334 /// | 0.853397013 |
| LOC553158 | NM_181335 | |
| ARHGDIB | NM_001175 | 1.05735295 |
| ASNS | NM_001673 /// NM_133436 /// NM_183356 | 0.868359418 |
| ATF3 | NM_001030287 /// NM_001674 /// | 1.759086651 |
| | NM_004024 | |
| ATP2A2 | NM_001681 /// NM_170665 | −1.067472852 |
| ATP6V0E | NM_003945 | 1.011503194 |
| AXL | NM_001699 /// NM_021913 | 0.922563085 |
| B3GNT3 | NM_014256 | 1.219829251 |
| B3GNT6 | NM_006876 | −1.716521904 |
| B4GALT6 | NM_004775 | −1.060570259 |
| BDKRB2 | NM_000623 | −1.376267776 |
| C10orf56 | NM_153367 | −1.650598054 |
| C1orf116 | NM_023938 | 1.312885916 |
| C1orf24 | NM_022083 /// NM_052966 | 1.28503906 |
| C8orf1 | NM_004337 | −0.808270443 |
| CA12 | NM_001218 /// NM_206925 | −0.913712636 |
| CA2 | NM_000067 | 1.089916815 |
| CACNA1G | NM_018896 /// NM_198376 /// NM_198377 | −1.276832883 |
| | /// | |
| | NM_198378 /// NM_198379 /// NM_198380 | |
| CASP7 | NM_001227 /// NM_033338 /// | 0.716947282 |
| | NM_033339 /// NM_033340 | |
| CCNG1 | NM_004060 /// NM_199246 | 0.895229961 |
| CDCP1 | NM_022842 /// NM_178181 | 1.340779747 |
| CDH1 | NM_004360 | 1.396526299 |
| CDS1 | NM_001263 | 2.316061732 |
| CEACAM6 | NM_002483 | 1.98336471 |
| CFH /// CFHL1 | NM_000186 /// NM_001014975 /// | −0.789194907 |
| | NM_002113 | |
| CGI-48 | NM_016001 | 0.782322175 |
| CLDN3 | NM_001306 | 1.073417052 |
| CRTAP | NM_006371 | −1.051122116 |
| CSPG2 | NM_004385 | −1.276229732 |
| CTGF | NM_001901 | 0.825095421 |
| CXCL1 | NM_001511 | 1.128627824 |
| CXCL2 | NM_002089 | 1.401048314 |
| CXCL3 | NM_002090 | 1.592782159 |
| CXCL5 | NM_002994 | 0.960535556 |
| CXX1 | NM_003928 | −1.128818951 |
| DAAM1 | NM_014992 | 1.031007263 |
| DAF | NM_000574 | 1.037132744 |
| DCAMKL1 | NM_004734 | 1.341038039 |
| DDAH1 | NM_012137 | 0.989731219 |
| DDC | NM_000790 | 1.259149649 |
| DICER1 | NM_030621 /// NM_177438 | 0.716895439 |
| DNAJB6 | NM_005494 /// NM_058246 | −0.794654919 |
| DNAJB9 | NM_012328 | 1.09578207 |
| DSC2 | NM_004949 /// NM_024422 | 1.690429678 |
| DSU | NM_018000 | 1.24874149 |
| DUSP5 | NM_004419 | 1.15862111 |
| DZIP1 | NM_014934 /// NM_198968 | −1.168010686 |
| EPLIN | NM_016357 | 1.238136451 |
| F11R | NM_016946 /// NM_144501 /// NM_144502 | 1.094438708 |
| | /// NM_144503 /// NM_144504 | |
| F5 | NM_000130 | 0.834127297 |
| FA2H | NM_024306 | 0.775822311 |
| FADS1 | NM_013402 | −1.42721961 |
| FAS | NM_000043 /// NM_152871 /// NM_152872 | 0.787212704 |
| | /// | |
| | NM_152873 /// NM_152874 /// NM_152875 | |
| FEZ2 | NM_005102 | −1.475084638 |
| FGB | NM_005141 | 1.093816564 |
| FGFBP1 | NM_005130 | 1.235082298 |
| FGFR4 | NM_002011 /// NM_022963 /// NM_213647 | −0.705326697 |
| FLJ11184 | NM_018352 | −1.220810548 |
| FLJ13910 | NM_022780 | 1.394622048 |
| FLJ20232 | NM_019008 | −1.07219661 |
| FN1 | NM_002026 /// NM_054034 /// NM_212474 | −1.359513905 |
| | /// | |
| | NM_212475 /// NM_212476 /// NM_212478 | |
| FNBP1 | NM_015033 | 1.001514783 |
| FSCN1 | NM_003088 | −0.725305455 |
| FSTL1 | NM_007085 | −0.78584492 |
| FXYD3 | NM_005971 /// NM_021910 | 1.654150293 |
| GALNT3 | NM_004482 | 2.249492952 |
| GATA6 | NM_005257 | 0.854525369 |
| GATM | NM_001482 | 0.820028622 |
| GCH1 | NM_000161 /// NM_001024024 /// | 1.202087236 |
| | NM_001024070 /// NM_001024071 | |
| GFPT1 | NM_002056 | 0.818168253 |
| GLI2 | NM_005270 /// NM_030379 /// NM_030380 | −1.278738148 |
| | /// NM_030381 | |
| GNA13 | NM_006572 | 1.011219061 |
| GNAS | NM_000516 /// NM_016592 /// NM_080425 | 1.138114266 |
| | /// NM_080426 | |
| GPR64 | NM_005756 | 0.889001537 |
| GREM1 | NM_013372 | 0.710816143 |
| H2AFY | NM_004893 /// NM_138609 /// NM_138610 | −1.352623135 |
| HIPK2 | NM_022740 | −1.053328106 |
| HMOX1 | NM_002133 | −0.749838973 |
| HPS5 | NM_007216 /// NM_181507 /// NM_181508 | −1.010452539 |
| HSPB8 | NM_014365 | 0.858706002 |
| HSPG2 | NM_005529 | −0.705327336 |
| IFIH1 | NM_022168 | 1.071093684 |
| IFRD1 | NM_001007245 /// NM_001550 | 1.016261255 |
| IGFBP1 | NM_000596 /// NM_001013029 | 0.951902406 |
| IGFBP4 | NM_001552 | −0.797667676 |
| IL11 | NM_000641 | −0.733031268 |
| IL32 | NM_001012631 /// NM_001012632 /// | 1.40247258 |
| | NM_001012633 | |
| | /// NM_001012634 /// NM_001012635 | |
| IL6 | NM_000600 | 0.773938846 |
| IL6R | NM_000565 /// NM_181359 | 1.218235824 |
| IL8 | NM_000584 | 1.216488232 |
| INHBC | NM_005538 | 0.754618311 |
| IPO7 | NM_006391 | −1.139922531 |
| ITGB4 | NM_000213 /// NM_001005619 /// | 0.724609877 |
| | NM_001005731 | |
| KCNK3 | NM_002246 | 1.055192637 |
| KCNMA1 | NM_001014797 /// NM_002247 | −0.887903486 |
| KCNS3 | NM_002252 | 1.190220199 |
| KDELC1 | NM_024089 | −1.861306446 |
| KIAA0485 | — | −0.819086376 |
| KIAA1164 | NM_019092 | −0.844281415 |
| KIAA1641 | NM_020970 | −0.949563346 |
| KLF4 | NM_004235 | 0.742260808 |
| KLHL24 | NM_017644 | 1.04021352 |
| KRT15 | NM_002275 | 1.371559465 |
| LAMB3 | NM_000228 /// NM_001017402 | 1.500692933 |
| LAMC2 | NM_005562 /// NM_018891 | 1.325222414 |
| LCN2 | NM_005564 | 1.501575887 |
| LEPR | NM_001003679 /// NM_001003680 /// | −1.167830731 |
| | NM_002303 | |
| LGALS8 | NM_006499 /// NM_201543 /// | 0.784434007 |
| | NM_201544 /// NM_201545 | |
| LHFP | NM_005780 | −1.198253378 |
| LISCH7 | NM_015925 /// NM_205834 /// NM_205835 | 1.750342418 |
| LOC153561 | NM_207331 | −0.814797607 |
| LOC348162 | XM_496132 | −1.180898446 |
| LOC440118 | XM_498554 | 1.153694936 |
| LUM | NM_002345 | 0.790696224 |
| MAFF | NM_012323 /// NM_152878 | 2.17862994 |
| MAP4K5 | NM_006575 /// NM_198794 | −0.804748402 |
| MARCKS | NM_002356 | −1.003360787 |
| MCFD2 | NM_139279 | −1.15440875 |
| MCL1 | NM_021960 /// NM_182763 | 1.157395536 |
| MCOLN3 | NM_018298 | 1.013954778 |
| ME1 | NM_002395 | −1.106251497 |
| MYO1D | NM_015194 | 1.649491344 |
| NCF2 | NM_000433 | 1.589521496 |
| NMU | NM_006681 | 0.944359891 |
| NPR3 | NM_000908 | 1.067325772 |
| NPTX1 | NM_002522 | −0.751618694 |
| NR5A2 | NM_003822 /// NM_205860 | −1.364250481 |
| NUCKS | NM_022731 | 1.011562834 |
| OLFML3 | NM_020190 | −0.730893288 |
| OSTM1 | NM_014028 | −2.503194824 |
| PCAF | NM_003884 | −0.954868141 |
| PCDH9 | NM_020403 /// NM_203487 | −0.752120619 |
| PDZK1 | NM_002614 | 1.976239117 |
| PGK1 | NM_000291 | 1.086525358 |
| PKP2 | NM_001005242 /// NM_004572 | 1.185009641 |
| PKP3 | NM_007183 | 0.954964696 |
| PLA2G12A | NM_030821 | 1.207835587 |
| PMCH | NM_002674 | 0.784044162 |
| PPIF | NM_005729 | 0.76107532 |
| PPL | NM_002705 | 1.322448059 |
| PPP1R15A | NM_014330 | 1.024293047 |
| PRSS16 | NM_005865 | 1.57042459 |
| PTGER4 | NM_000958 | 1.196243588 |
| QKI | NM_006775 /// NM_206853 /// | −2.723444139 |
| | NM_206854 /// NM_206855 | |
| RAB11FIP2 | NM_014904 | −1.313369214 |
| RAB2 | NM_002865 | 0.905943562 |
| RAFTLIN | NM_015150 | −0.877153315 |
| RAP140 | NM_015224 | −1.500817539 |
| RARRES1 | NM_002888 /// NM_206963 | −0.829191364 |
| RASGRP1 | NM_005739 | 1.531443906 |
| RBL1 | NM_002895 /// NM_183404 | −1.041533883 |
| RBM35A | NM_001034915 /// NM_017697 | 2.221571941 |
| RBP4 | NM_006744 | 0.77127411 |
| RECK | NM_021111 | −1.403541796 |
| RGC32 | NM_014059 | 1.30856501 |
| RHEB | NM_005614 | 0.832682259 |
| RHOB | NM_004040 | 0.940974974 |
| RLN2 | NM_005059 /// NM_134441 | 1.44357207 |
| RP2 | NM_006915 | 0.866057767 |
| RPL38 | NM_000999 | 0.910678855 |
| S100P | NM_005980 | 0.736600264 |
| SAMD4 | NM_015589 | 0.956912129 |
| SC4MOL | NM_001017369 /// NM_006745 | −0.709894592 |
| SCD | NM_005063 | −0.751403822 |
| SCEL | NM_003843 /// NM_144777 | 2.019902319 |
| SE57-1 | NM_025214 | 0.989125512 |
| SEC23A | NM_006364 | −1.276322792 |
| SELENBP1 | NM_003944 | −0.778818613 |
| SEPT6 | NM_015129 /// NM_145799 /// NM_145800 | −1.144884272 |
| | /// NM_145802 | |
| SFRP4 | NM_003014 | −1.133063935 |
| SHCBP1 | NM_024745 | −1.333766441 |
| SLC11A2 | NM_000617 | 0.778653795 |
| SLC1A4 | NM_003038 | 0.873687285 |
| SLC2A3 | NM_006931 | 1.43767315 |
| SLC2A3/SLC2A14 | NM_006931 /// NM_153449 | 1.535130121 |
| SMA4 | NM_021652 | −0.941969174 |
| SOCS2 | NM_003877 | 1.21852495 |
| SOD2 | NM_000636 /// NM_001024465 /// | 0.714304877 |
| | NM_001024466 | |
| SOX18 | NM_018419 | 2.396912781 |
| SPARC | NM_003118 | −0.844033461 |
| SPHAR | NM_006542 | −1.200461954 |
| SPINT1 | NM_001032367 /// NM_003710 /// | 2.044323684 |
| | NM_181642 | |
| SRD5A1 | NM_001047 | 0.850521639 |
| SRPX | NM_006307 | 0.776360306 |
| ST7 | NM_018412 /// NM_021908 | 0.80619458 |
| STC1 | NM_003155 | 0.904730168 |
| STC2 | NM_003714 | 1.474977835 |
| STX3A | NM_004177 | 1.367928944 |
| STYK1 | NM_018423 | 1.10910972 |
| SUMO2 | NM_001005849 /// NM_006937 | 0.746286447 |
| SWAP70 | NM_015055 | −1.130416982 |
| SYDE1 | NM_033025 | −1.03693344 |
| TACSTD1 | NM_002354 | 3.752570657 |
| TCF8 | NM_030751 | −1.772963376 |
| TDO2 | NM_005651 | −0.717287845 |
| TJP2 | NM_004817 /// NM_201629 | 1.435012945 |
| TMEM45A | NM_018004 | −1.18663334 |
| TNFAIP6 | NM_007115 | −1.243508842 |
| TNRC9 | XM_049037 | 1.108269071 |
| TRA1 | NM_003299 | 1.300897339 |
| TRIB3 | NM_021158 | 1.113526734 |
| TTC9 | XM_027236 | 1.165031136 |
| TTMP | NM_024616 | 1.133320077 |
| TUBB4 | NM_006087 | −0.704131434 |
| TXN | NM_003329 | 1.165870308 |
| UGT1A8/UGT1A9 | NM_019076 /// NM_021027 | −0.821829527 |
| VAMP8 | NM_003761 | 1.501535152 |
| VAV3 | NM_006113 | −0.701108757 |
| VIL1 | NM_007127 | 1.92182874 |
| VIL2 | NM_003379 | 0.717349426 |
| WASPIP | NM_003387 | −1.17434511 |
| ZBED2 | NM_024508 | 2.422626946 |
| ZFHX1B | NM_014795 | −1.221728077 |
| ZNF165 | NM_003447 | 3.308802789 |

A further embodiment of the invention is directed to methods of modulating a cellular pathway comprising administering to the cell an amount of an isolated nucleic acid comprising a miR-200 nucleic acid sequence or a miR-200 inhibitor. A cell, tissue, or subject may be a cancer cell, a cancerous tissue or harbor cancerous tissue, or a cancer patient. The database content related to all nucleic acids and genes designated by an accession number or a database submission are incorporated herein by reference as of the filing date of this application.

A further embodiment of the invention is directed to methods of modulating a cellular pathway comprising administering to the cell an amount of an isolated nucleic acid comprising a miR-200 nucleic acid sequence in an amount sufficient to modulate the expression, function, status, or state of a cellular pathway, in particular those pathways described in Table 2 or the pathways known to include one or more genes from Table 1, 3, 4, and/or 5. Modulation of a cellular pathway includes, but is not limited to modulating the expression of one or more gene(s). Modulation of a gene can include inhibiting the function of an endogenous miRNA or providing a functional miRNA to a cell, tissue, or subject. Modulation refers to the expression levels or activities of a gene or its related gene product (e.g., mRNA) or protein, e.g., the mRNA levels may be modulated or the translation of an mRNA may be modulated. Modulation may increase or up regulate a gene or gene product or it may decrease or down regulate a gene or gene product (e.g., protein levels or activity).

Still a further embodiment includes methods of administering an miRNA or mimic thereof, and/or treating a subject or patient having, suspected of having, or at risk of developing a pathological condition comprising one or more of step (a) administering to a patient or subject an amount of an isolated nucleic acid comprising a miR-200 nucleic acid sequence or a miR-200 inhibitor in an amount sufficient to modulate expression of a cellular pathway; and (b) administering a second therapy, wherein the modulation of the cellular pathway sensitizes the patient or subject, or increases the efficacy of a second therapy. An increase in efficacy can include a reduction in toxicity, a reduced dosage or duration of the second therapy, or an additive or synergistic effect. A cellular pathway may include, but is not limited to one or more pathway described in Table 2 below or a pathway that is know to include one or more genes of Tables 1, 3, 4, and/or 5. The second therapy may be administered before, during, and/or after the isolated nucleic acid or miRNA or inhibitor is administered

A second therapy can include administration of a second miRNA or therapeutic nucleic acid such as a siRNA or antisense oligonucleotide, or may include various standard therapies, such as pharmaceuticals, chemotherapy, radiation therapy, drug therapy, immunotherapy, and the like. Embodiments of the invention may also include the determination or assessment of gene expression or gene expression profile for the selection of an appropriate therapy. In a particular aspect, a second therapy is a chemotherapy. A chemotherapy can include, but is not limited to paclitaxel, cisplatin, carboplatin, doxorubicin, oxaliplatin, larotaxel, taxol, lapatinib, docetaxel, methotrexate, capecitabine, vinorelbine, cyclophosphamide, gemcitabine, amrubicin, cytarabine, etoposide, camptothecin, dexamethasone, dasatinib, tipifarnib, bevacizumab, sirolimus, temsirolimus, everolimus, lonafarnib, cetuximab, erlotinib, gefitinib, imatinib mesylate, rituximab, trastuzumab, nocodazole, sorafenib, sunitinib, bortezomib, alemtuzumab, gemtuzumab, tositumomab or ibritumomab.

Embodiments of the invention include methods of treating a subject with a disease or condition comprising one or more of the steps of (a) determining an expression profile of one or more genes selected from Table 1, 3, 4, and/or 5; (b) assessing the sensitivity of the subject to therapy based on the expression profile; (c) selecting a therapy based on the assessed sensitivity; and (d) treating the subject using a selected therapy. Typically, the disease or condition will have as a component, indicator, or resulting mis-regulation of one or more gene of Table 1, 3, 4, and/or 5.

In certain aspects, 2, 3, 4, 5, 6, 7, 8, 9, 10, or more miRNA may be used in sequence or in combination; for instance, any combination of miR-200 or a miR-200 inhibitor with another miRNA. Further embodiments include the identification and assessment of an expression profile indicative of miR-200 status in a cell or tissue comprising expression assessment of one or more gene from Table 1, 3, 4, and/or 5, or any combination thereof.

The term “miRNA” is used according to its ordinary and plain meaning and refers to a microRNA molecule found in eukaryotes that is involved in RNA-based gene regulation. See, e.g., Carrington et al., 2003, which is hereby incorporated by reference. The term can be used to refer to the single-stranded RNA molecule processed from a precursor or in certain instances the precursor itself.

In some embodiments, it may be useful to know whether a cell expresses a particular miRNA endogenously or whether such expression is affected under particular conditions or when it is in a particular disease state. Thus, in some embodiments of the invention, methods include assaying a cell or a sample containing a cell for the presence of one or more marker gene or mRNA or other analyte indicative of the expression level of a gene of interest. Consequently, in some embodiments, methods include a step of generating an RNA profile for a sample. The term “RNA profile” or “gene expression profile” refers to a set of data regarding the expression pattern for one or more gene or genetic marker or miRNA in the sample (e.g., a plurality of nucleic acid probes that identify one or more markers from Tables 1, 3, 4, and/or 5); it is contemplated that the nucleic acid profile can be obtained using a set of RNAs, using for example nucleic acid amplification or hybridization techniques well know to one of ordinary skill in the art. The difference in the expression profile in the sample from the patient and a reference expression profile, such as an expression profile of one or more genes or miRNAs, are indicative of which miRNAs to be administered.

In certain aspects, miR-200 or miR-200 inhibitor and let-7 can be administered to patients with breast carcinoma, cervical carcinoma, chronic lymphoblastic leukemia, colorectal carcinoma, glioma, glioblastoma, gastric carcinoma, hepatocellular carcinoma, leukemia, lung carcinoma, multiple myeloma, non-small cell lung carcinoma, ovarian carcinoma, oesophageal carcinoma, pancreatic carcinoma, prostate carcinoma, squamous cell carcinoma of the head and neck, thyroid carcinoma.

Further aspects include administering miR-200 or miR-200 inhibitor and miR-15 to patients with breast carcinoma, B-cell lymphoma, cervical carcinoma, colorectal carcinoma, glioma, glioblastoma, gastric carcinoma, hepatocellular carcinoma, lung carcinoma, multiple myeloma, non-small cell lung carcinoma, ovarian carcinoma, oesophageal carcinoma, pancreatic carcinoma, prostate carcinoma, rhabdomyosarcoma, squamous cell carcinoma of the head and neck, thyroid carcinoma.

In still further aspects, miR-200 or miR-200 inhibitor and miR-16 are administered to patients with breast carcinoma, B-cell lymphoma, colorectal carcinoma, glioblastoma, gastric carcinoma, hepatocellular carcinoma, multiple myeloma, non-small cell lung carcinoma, ovarian carcinoma, oesophageal carcinoma, pancreatic carcinoma, prostate carcinoma, rhabdomyosarcoma, squamous cell carcinoma of the head and neck, thyroid carcinoma.

In certain aspects, miR-200 or miR-200 inhibitor and miR-20 are administered to patients with breast carcinoma, cervical carcinoma, colorectal carcinoma, glioma, glioblastoma, gastric carcinoma, hepatocellular carcinoma leukemia, lipoma, multiple myeloma, non-small cell lung carcinoma, ovarian carcinoma, oesophageal carcinoma, osteosarcoma, pancreatic carcinoma, prostate carcinoma, squamous cell carcinoma of the head and neck, thyroid carcinoma.

Aspects of the invention include methods where miR-200 or miR-200 inhibitor and miR-21 are administered to patients with breast carcinoma, colorectal carcinoma, glioma, glioblastoma, gastric carcinoma, hepatocellular carcinoma, non-small cell lung carcinoma, ovarian carcinoma, oesophageal carcinoma, pancreatic carcinoma, prostate carcinoma, rhabdomyosarcoma, squamous cell carcinoma of the head and neck.

In still further aspects, miR-200 or miR-200 inhibitor and miR-26a are administered to patients with anaplastic large cell lymphoma, breast carcinoma, B-cell lymphoma, cervical carcinoma, chronic lymphoblastic leukemia, colorectal carcinoma, glioma, glioblastoma, gastric carcinoma, hepatocellular carcinoma, leukemia, lung carcinoma, multiple myeloma, non-small cell lung carcinoma, ovarian carcinoma, oesophageal carcinoma, osteosarcoma, pancreatic carcinoma, prostate carcinoma, rhabdomyosarcoma, testicular tumor.

In yet a further aspect, miR-200 or miR-200 inhibitor and miR-34a are administered to patients with anaplastic large cell lymphoma, breast carcinoma, B-cell lymphoma, cervical carcinoma, chronic lymphoblastic leukemia, colorectal carcinoma, glioma, glioblastoma, gastric carcinoma, hepatocellular carcinoma, leukemia, lung carcinoma, multiple myeloma, mesothelioma, non-small cell lung carcinoma, ovarian carcinoma, oesophageal carcinoma, osteosarcoma, pancreatic carcinoma, prostate carcinoma, rhabdomyosarcoma, squamous cell carcinoma of the head and neck, thyroid carcinoma, testicular tumor.

In yet further aspects, miR-200 or miR-200 inhibitor and miR-126 are administered to patients with breast carcinoma, cervical carcinoma, colorectal carcinoma, glioma, glioblastoma, gastric carcinoma, hepatocellular carcinoma, leukemia, lung carcinoma, mesothelioma, non-small cell lung carcinoma, ovarian carcinoma, oesophageal carcinoma, osteosarcoma, pancreatic carcinoma, prostate carcinoma, rhabdomyosarcoma, squamous cell carcinoma of the head and neck, thyroid carcinoma.

In a further aspect, miR-200 or miR-200 inhibitor and miR-143 are administered to patients with anaplastic large cell lymphoma, breast carcinoma, B-cell lymphoma, cervical carcinoma, chronic lymphoblastic leukemia, colorectal carcinoma, glioma, glioblastoma, gastric carcinoma, hepatocellular carcinoma, leukemia, lung carcinoma, multiple myeloma, non-small cell lung carcinoma, ovarian carcinoma, oesophageal carcinoma, osteosarcoma, pancreatic carcinoma, prostate carcinoma, squamous cell carcinoma of the head and neck, thyroid carcinoma, testicular tumor.

In still a further aspect, miR-200 or miR-200 inhibitor and miR-147 are administered to patients with breast carcinoma, cervical carcinoma, colorectal carcinoma, glioma, glioblastoma, gastric carcinoma, hepatocellular carcinoma, leukemia, lipoma, multiple myeloma, non-small cell lung carcinoma, ovarian carcinoma, oesophageal carcinoma, osteosarcoma, pancreatic carcinoma, prostate carcinoma, squamous cell carcinoma of the head and neck, thyroid carcinoma.

In yet another aspect, miR-200 or miR-200 inhibitor and miR-188 are administered to patients with anaplastic large cell lymphoma, breast carcinoma, B-cell lymphoma, cervical carcinoma, chronic lymphoblastic leukemia, colorectal carcinoma, glioma, glioblastoma, gastric carcinoma, hepatocellular carcinoma, leukemia, lung carcinoma, multiple myeloma, non-small cell lung carcinoma, ovarian carcinoma, oesophageal carcinoma, pancreatic carcinoma, prostate carcinoma, squamous cell carcinoma of the head and neck, thyroid carcinoma, testicular tumor.

In other aspects, miR-200 or miR-200 inhibitor and miR-215 are administered to patients with anaplastic large cell lymphoma, breast carcinoma, B-cell lymphoma, cervical carcinoma, chronic lymphoblastic leukemia, colorectal carcinoma, glioma, glioblastoma, gastric carcinoma, hepatocellular carcinoma, leukemia, lung carcinoma, lipoma, multiple myeloma, mesothelioma, non-small cell lung carcinoma, ovarian carcinoma, oesophageal carcinoma, osteosarcoma, pancreatic carcinoma, prostate carcinoma, rhabdomyosarcoma, squamous cell carcinoma of the head and neck, thyroid carcinoma, testicular tumor.

In certain aspects, miR-200 or miR-200 inhibitor and miR-216 are administered to patients with breast carcinoma, cervical carcinoma, colorectal carcinoma, glioma, glioblastoma, gastric carcinoma, hepatocellular carcinoma, leukemia, lung carcinoma, non-small cell lung carcinoma, ovarian carcinoma, oesophageal carcinoma, osteosarcoma, prostate carcinoma, squamous cell carcinoma of the head and neck, testicular tumor.

In a further aspect, miR-200 or miR-200 inhibitor and miR-292-3p are administered to patients with anaplastic large cell lymphoma, breast carcinoma, B-cell lymphoma, cervical carcinoma, colorectal carcinoma, glioma, glioblastoma, gastric carcinoma, hepatocellular carcinoma, leukemia, lung carcinoma, lipoma, multiple myeloma, non-small cell lung carcinoma, ovarian carcinoma, oesophageal carcinoma, osteosarcoma, pancreatic carcinoma, prostate carcinoma, rhabdomyosarcoma, squamous cell carcinoma of the head and neck, thyroid carcinoma, testicular tumor.

In still a further aspect, miR-200 or miR-200 inhibitor and miR-331 are administered to patients with anaplastic large cell lymphoma, breast carcinoma, B-cell lymphoma, cervical carcinoma, chronic lymphoblastic leukemia, colorectal carcinoma, glioma, glioblastoma, gastric carcinoma, hepatocellular carcinoma, leukemia, lung carcinoma, multiple myeloma, ovarian carcinoma, oesophageal carcinoma, osteosarcoma, pancreatic carcinoma, prostate carcinoma, rhabdomyosarcoma, squamous cell carcinoma of the head and neck, thyroid carcinoma, testicular tumor.

It is contemplated that when miR-200 or a miR-200 inhibitor is given in combination with one or more other miRNA molecules, the two different miRNAs or inhibitors may be given at the same time or sequentially. In some embodiments, therapy proceeds with one miRNA or inhibitor and that therapy is followed up with therapy with the other miRNA or inhibitor 1, 2, 3, 4, 5, 6, 7, 8, 9, 10, 15, 20, 25, 30, 35, 40, 45, 50, 55 minutes, 1, 2, 3, 4, 5, 6, 7, 8, 9, 10, 11, 12, 13, 14, 15, 16, 17, 18, 19, 20, 21, 22, 23, 24 hours, 1, 2, 3, 4, 5, 6, 7 days, 1, 2, 3, 4, 5 weeks, or 1, 2, 3, 4, 5, 6, 7, 8, 9, 10, 11, or 12 months or any such combination later.

Further embodiments include the identification and assessment of an expression profile indicative of miR-200 status in a cell or tissue comprising expression assessment of one or more gene from Table 1, 3, 4, and/or 5, or any combination thereof.

The term “miRNA” is used according to its ordinary and plain meaning and refers to a microRNA molecule found in eukaryotes that is involved in RNA-based gene regulation. See, e.g., Carrington et al., 2003, which is hereby incorporated by reference. The term can be used to refer to the single-stranded RNA molecule processed from a precursor or in certain instances the precursor itself or a mimetic thereof.

In some embodiments, it may be useful to know whether a cell expresses a particular miRNA endogenously or whether such expression is affected under particular conditions or when it is in a particular disease state. Thus, in some embodiments of the invention, methods include assaying a cell or a sample containing a cell for the presence of one or more miRNA marker gene or mRNA or other analyte indicative of the expression level of a gene of interest. Consequently, in some embodiments, methods include a step of generating an RNA profile for a sample. The term “RNA profile” or “gene expression profile” refers to a set of data regarding the expression pattern for one or more gene or genetic marker in the sample (e.g., a plurality of nucleic acid probes that identify one or more markers or genes from Tables 1, 3, 4, and/or 5); it is contemplated that the nucleic acid profile can be obtained using a set of RNAs, using for example nucleic acid amplification or hybridization techniques well know to one of ordinary skill in the art. The difference in the expression profile in the sample from a patient and a reference expression profile, such as an expression profile from a normal or non-pathologic sample, or a digitized reference, is indicative of a pathologic, disease, or cancerous condition. In certain aspects the expression profile is an indicator of a propensity to or probability of (i.e., risk factor for a disease or condition) developing such a condition(s). Such a risk or propensity may indicate a treatment, increased monitoring, prophylactic measures, and the like. A nucleic acid or probe set may comprise or identify a segment of a corresponding mRNA and may include all or part of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 100, 200, 500, or more segments, including any integer or range derivable there between, of a gene or genetic marker, or a nucleic acid, mRNA or a probe representative thereof that is listed in Tables 1, 3, 4, and/or 5 or identified by the methods described herein.

Certain embodiments of the invention are directed to compositions and methods for assessing, prognosing, or treating a pathological condition in a patient comprising measuring or determining an expression profile of one or more miRNA or marker(s) in a sample from the patient, wherein a difference in the expression profile in the sample from the patient and an expression profile of a normal sample or reference expression profile is indicative of pathological condition and particularly cancer (e.g., In certain aspects of the invention, the miRNAs, cellular pathway, gene, or genetic marker is or is representative of one or more pathway or marker described in Table 1, 2, 3, 4, and/or 5, including any combination thereof.

Aspects of the invention include diagnosing, assessing, or treating a pathologic condition or preventing a pathologic condition from manifesting. For example, the methods can be used to screen for a pathological condition; assess prognosis of a pathological condition; stage a pathological condition; assess response of a pathological condition to therapy; or to modulate the expression of a gene, genes, or related pathway as a first therapy or to render a subject sensitive or more responsive to a second therapy. In particular aspects, assessing the pathological condition of the patient can be assessing prognosis of the patient. Prognosis may include, but is not limited to an estimation of the time or expected time of survival, assessment of response to a therapy, and the like. In certain aspects, the altered expression of one or more gene or marker is prognostic for a patient having a pathologic condition, wherein the marker is one or more of Table 1, 3, 4, and/or 5, including any combination thereof.

TABLE 2
Significantly affected functional cellular pathways following
hsa-miR-200 over-expression in human cancer cells.

| Number | |
|---|---|
| of Genes | Pathway Functions |
| 18 | Dermatological Diseases and Conditions, Genetic Disorder, |
| | Cardiovascular Disease |
| 15 | Cellular Movement, Hematological System Development |
| | and Function, Immune Response |
| 13 | Cellular Movement, Cell Morphology, Cellular Development |
| 13 | Cellular Movement, Embryonic Development, Carbohydrate |
| | Metabolism |
| 13 | Cell-To-Cell Signaling and Interaction, Tissue Development, |
| | Cellular Movement |
| 11 | Cancer, Cellular Growth and Proliferation, Reproductive |
| | System Disease |
| 11 | Cellular Function and Maintenance, Cellular Assembly and |
| | Organization, Drug Metabolism |
| 4 | Cellular Growth and Proliferation, Hematological System |
| | Development and Function, Immune Response |
| 1 | Cell Morphology, Cellular Assembly and Organization, |
| | Psychological Disorders |
| 1 | Genetic Disorder, Hematological Disease, Post-Translational |
| | Modification |
| 1 | Cancer, Cellular Growth and Proliferation, Ophthalmic |
| | Disease |
| 1 | Genetic Disorder, Cellular Assembly and Organization |
| 1 | Molecular Transport, Protein Trafficking, Cell-To-Cell |
| | Signaling and Interaction |

TABLE 3
Predicted target genes of hsa-miR-200 for Ref Seq ID reference - Pruitt et al., 2005.

| | Ref Seq | |
|---|---|---|
| | (Pruitt et al., | |
| Gene Symbol | 2005) | Description |
| 2′-PDE | NM_177966 | 2′-phosphodiesterase |
| A1BG | NM_130786 | alpha 1B-glycoprotein |
| A2BP1 | NM_145891 | ataxin 2-binding protein 1 isoform 1 |
| AADACL1 | NM_020792 | arylacetamide deacetylase-like 1 |
| AASDHPPT | NM_015423 | aminoadipate-semialdehyde |
| ABAT | NM_000663 | 4-aminobutyrate aminotransferase precursor |
| ABCA13 | NM_152701 | ATP binding cassette, sub-family A (ABC1), |
| ABCA9 | NM_080283 | ATP-binding cassette, sub-family A, member 9 |
| ABCB10 | NM_012089 | ATP-binding cassette, sub-family B, member |
| | | 10 |
| ABCC13 | NM_138726 | ATP-binding cassette protein C13 isoform a |
| ABCD3 | NM_002858 | ATP-binding cassette, sub-family D, member 3 |
| ABI2 | NM_005759 | abl interactor 2 |
| ACACA | NM_198834 | acetyl-Coenzyme A carboxylase alpha isoform 1 |
| ACADSB | NM_001609 | acyl-Coenzyme A dehydrogenase, |
| | | short/branched |
| ACE2 | NM_021804 | angiotensin I converting enzyme 2 precursor |
| ACOT8 | NM_183385 | peroxisomal acyl-CoA thioesterase 1 isoform b |
| ACRC | NM_052957 | ACRC protein |
| ACSL5 | NM_016234 | acyl-CoA synthetase long-chain family |
| | | member 5 |
| ACTR3 | NM_005721 | ARP3 actin-related protein 3 homolog |
| ACVR1C | NM_145259 | activin A receptor, type IC |
| ACVR2A | NM_001616 | activin A receptor, type IIA precursor |
| ACY1L2 | NM_001010853 | hypothetical protein LOC135293 |
| ADAM12 | NM_003474 | ADAM metallopeptidase domain 12 isoform 1 |
| ADAMDEC1 | NM_014479 | ADAM-like, decysin 1 |
| ADAMTS3 | NM_014243 | ADAM metallopeptidase with |
| | | thrombospondin type 1 |
| ADAMTS5 | NM_007038 | ADAM metallopeptidase with |
| | | thrombospondin type 1 |
| ADAMTS9 | NM_182920 | ADAM metallopeptidase with |
| | | thrombospondin type 1 |
| ADARB1 | NM_001112 | RNA-specific adenosine deaminase B1 |
| | | isoform 1 |
| ADCY9 | NM_001116 | adenylate cyclase 9 |
| ADD3 | NM_001121 | adducin 3 (gamma) isoform b |
| ADH1B | NM_000668 | alcohol dehydrogenase 1B (class I), beta |
| ADIPOR2 | NM_024551 | adiponectin receptor 2 |
| ADRB2 | NM_000024 | adrenergic, beta-2-, receptor, surface |
| AES | NM_001130 | amino-terminal enhancer of split isoform b |
| AFF1 | NM_005935 | myeloid/lymphoid or mixed-lineage leukemia |
| AFF3 | NM_001025108 | AF4/FMR2 family, member 3 isoform 2 |
| AGBL3 | NM_178563 | ATP/GTP binding protein-like 3 |
| AKAP13 | NM_006738 | A-kinase anchor protein 13 isoform 1 |
| AKAP6 | NM_004274 | A-kinase anchor protein 6 |
| AKAP7 | NM_004842 | A-kinase anchor protein 7 isoform alpha |
| AKT3 | NM_005465 | v-akt murine thymoma viral oncogene |
| | | homolog 3 |
| ALCAM | NM_001627 | activated leukocyte cell adhesion molecule |
| ALDH1A3 | NM_000693 | aldehyde dehydrogenase 1A3 |
| ALG8 | NM_001007027 | asparagine-linked glycosylation 8 isoform b |
| ALS2CR14 | NM_178231 | amyotrophic lateral sclerosis 2 (juvenile) |
| ALS2CR15 | NM_138468 | Ica69-related protein |
| ALS2CR19 | NM_057177 | amyotrophic lateral sclerosis 2 (juvenile) |
| ALS2CR8 | NM_024744 | amyotrophic lateral sclerosis 2 (juvenile) |
| AMFR | NM_001144 | autocrine motility factor receptor |
| AMOTL1 | NM_130847 | angiomotin like 1 |
| AMOTL2 | NM_016201 | angiomotin like 2 |
| AMPD3 | NM_000480 | erythrocyte adenosine monophosphate |
| | | deaminase |
| ANGPTL1 | NM_004673 | angiopoietin-like 1 precursor |
| ANK3 | NM_001149 | ankyrin 3 isoform 2 |
| ANKFY1 | NM_020740 | ankyrin repeat and FYVE domain containing 1 |
| ANKH | NM_054027 | ankylosis, progressive homolog |
| ANKMY2 | NM_020319 | ankyrin repeat and MYND domain containing 2 |
| ANKRD19 | NM_001010925 | ankyrin repeat domain 19 |
| ANKRD25 | NM_015493 | ankyrin repeat domain 25 |
| ANKRD27 | NM_032139 | ankyrin repeat domain 27 (VPS9 domain) |
| ANKRD28 | NM_015199 | ankyrin repeat domain 28 |
| ANKRD40 | NM_052855 | hypothetical protein LOC91369 |
| ANKRD42 | NM_182603 | ankyrin repeat domain 42 |
| ANKRD44 | NM_153697 | hypothetical protein DKFZp434D2328 |
| ANKRD46 | NM_198401 | ankyrin repeat domain 46 |
| ANKZF1 | NM_018089 | ankyrin repeat and zinc finger domain |
| | | containing |
| ANLN | NM_018685 | anillin, actin binding protein (scraps homolog, |
| ANXA7 | NM_001156 | annexin VII isoform 1 |
| AOF1 | NM_153042 | amine oxidase (flavin containing) domain 1 |
| AP1GBP1 | NM_007247 | AP1 gamma subunit binding protein 1 isoform 1 |
| AP1M1 | NM_032493 | adaptor-related protein complex 1, mu 1 |
| | | subunit |
| AP1S2 | NM_003916 | adaptor-related protein complex 1 sigma 2 |
| AP4S1 | NM_007077 | adaptor-related protein complex 4, sigma 1 |
| APAF1 | NM_001160 | apoptotic protease activating factor isoform b |
| APLP2 | NM_001642 | amyloid beta (A4) precursor-like protein 2 |
| APOC3 | NM_000040 | apolipoprotein C-III precursor |
| APRIN | NM_015032 | androgen-induced prostate proliferative |
| | | shutoff |
| APXL | NM_001649 | apical protein of Xenopus-like |
| ARHGAP11A | NM_014783 | Rho GTPase activating protein 11A isoform 1 |
| ARHGAP18 | NM_033515 | Rho GTPase activating protein 18 |
| ARHGAP19 | NM_032900 | Rho GTPase activating protein 19 |
| ARHGAP20 | NM_020809 | Rho GTPase activating protein 20 |
| ARHGAP28 | NM_001010000 | Rho GTPase activating protein 28 isoform a |
| ARHGAP5 | NM_001030055 | Rho GTPase activating protein 5 isoform a |
| ARHGAP6 | NM_001174 | Rho GTPase activating protein 6 isoform 2 |
| ARHGDIA | NM_004309 | Rho GDP dissociation inhibitor (GDI) alpha |
| ARHGEF1 | NM_004706 | Rho guanine nucleotide exchange factor 1 |
| | | isoform |
| ARHGEF10 | NM_014629 | Rho guanine nucleotide exchange factor 10 |
| ARHGEF12 | NM_015313 | Rho guanine nucleotide exchange factor |
| | | (GEF) 12 |
| ARHGEF3 | NM_019555 | Rho guanine nucleotide exchange factor 3 |
| ARHGEF6 | NM_004840 | Rac/Cdc42 guanine nucleotide exchange factor 6 |
| ARHGEF7 | NM_145735 | Rho guanine nucleotide exchange factor 7 |
| | | isoform |
| ARHGEF9 | NM_015185 | Cdc42 guanine exchange factor 9 |
| ARID2 | NM_152641 | AT rich interactive domain 2 (ARID, RFX- |
| | | like) |
| ARIH2 | NM_006321 | ariadne homolog 2 |
| ARL1 | NM_001177 | ADP-ribosylation factor-like 1 |
| ARL10 | NM_173664 | ADP-ribosylation factor-like 10 |
| ARL8B | NM_018184 | ADP-ribosylation factor-like 10C |
| ARRDC4 | NM_183376 | arrestin domain containing 4 |
| ARSD | NM_009589 | arylsulfatase D isoform b precursor |
| ARSJ | NM_024590 | arylsulfatase J |
| ASB1 | NM_016114 | ankyrin repeat and SOCS box-containing |
| | | protein |
| ASB13 | NM_024701 | ankyrin repeat and SOCS box-containing |
| | | protein |
| ASB5 | NM_080874 | ankyrin repeat and SOCS box-containing |
| | | protein |
| ASCC3 | NM_006828 | activating signal cointegrator 1 complex |
| | | subunit |
| ASCIZ | NM_015251 | ATM/ATR-Substrate Chk2-Interacting Zn2+- |
| | | finger |
| ASF1A | NM_014034 | ASF1 anti-silencing function 1 homolog A |
| ASPN | NM_017680 | asporin (LRR class 1) |
| ASTN2 | NM_014010 | astrotactin 2 isoform a |
| ASXL1 | NM_015338 | additional sex combs like 1 |
| ATF7 | NM_006856 | activating transcription factor 7 |
| ATP10A | NM_024490 | ATPase, Class V, type 10A |
| ATP11A | NM_015205 | ATPase, Class VI, type 11A isoform a |
| ATP11B | NM_014616 | ATPase, Class VI, type 11B |
| ATP11C | NM_001010986 | ATPase, Class VI, type 11C isoform b |
| ATP2A2 | NM_170665 | ATPase, Ca++ transporting, cardiac muscle, |
| | | slow |
| ATP2C1 | NM_001001485 | calcium-transporting ATPase 2C1 isoform 1c |
| ATP6V1A | NM_001690 | ATPase, H+ transporting, lysosomal 70 kD, V1 |
| ATP6V1E1 | NM_001696 | vacuolar H+ ATPase E1 isoform a |
| ATPBD4 | NM_080650 | ATP binding domain 4 |
| ATRX | NM_000489 | transcriptional regulator ATRX isoform 1 |
| ATXN1 | NM_000332 | ataxin 1 |
| AXIN2 | NM_004655 | axin 2 |
| B3GALNT1 | NM_033167 | UDP-Gal:betaGlcNAc beta |
| B3GALTL | NM_194318 | beta 3-glycosyltransferase-like |
| B3GAT1 | NM_018644 | beta-1,3-glucuronyltransferase 1 |
| B3GNT1 | NM_006876 | UDP-GlcNAc:betaGal |
| B3GNT2 | NM_006577 | UDP-GlcNAc:betaGal |
| B4GALT6 | NM_004775 | UDP-Gal:betaGlcNAc beta 1,4- |
| bA16L21.2.1 | NM_001015882 | hypothetical protein LOC548645 |
| BAALC | NM_001024372 | brain and acute leukemia, cytoplasmic isoform 2 |
| BACH2 | NM_021813 | BTB and CNC homology 1, basic leucine |
| | | zipper |
| BAG4 | NM_004874 | BCL2-associated athanogene 4 |
| BAGE | NM_001187 | B melanoma antigen |
| BAP1 | NM_004656 | BRCA1 associated protein-1 |
| BASP1 | NM_006317 | brain abundant, membrane attached signal |
| | | protein |
| BAT2D1 | NM_015172 | HBxAg transactivated protein 2 |
| BAT3 | NM_004639 | HLA-B associated transcript-3 isoform a |
| BATF | NM_006399 | basic leucine zipper transcription factor, |
| BCAP29 | NM_001008405 | B-cell receptor-associated protein BAP29 |
| | | isoform |
| BCL11B | NM_022898 | B-cell CLL/lymphoma 11B isoform 2 |
| BCL2 | NM_000633 | B-cell lymphoma protein 2 alpha isoform |
| BCL2L11 | NM_006538 | BCL2-like 11 isoform 6 |
| BCLAF1 | NM_014739 | BCL2-associated transcription factor 1 |
| BDKRB2 | NM_000623 | bradykinin receptor B2 |
| BET1 | NM_005868 | blocked early in transport 1 |
| BHLHB3 | NM_030762 | basic helix-loop-helix domain containing, |
| | | class |
| BHLHB5 | NM_152414 | basic helix-loop-helix domain containing, |
| | | class |
| BHMT | NM_001713 | betaine-homocysteine methyltransferase |
| BICD2 | NM_001003800 | bicaudal D homolog 2 isoform 1 |
| BIRC1 | NM_004536 | baculoviral IAP repeat-containing 1 |
| BMI1 | NM_005180 | polycomb group ring finger 4 |
| BMPER | NM_133468 | BMP-binding endothelial regulator precursor |
| BNC2 | NM_017637 | basonuclin 2 |
| BOLL | NM_033030 | boule isoform 2 |
| BPY2 | NM_004678 | variable charge, Y chromosome, 2 protein |
| BPY2B | NM_001002760 | basic charge, Y-linked, 2B |
| BPY2C | NM_001002761 | basic charge, Y-linked, 2C |
| BRCA1 | NM_007306 | breast cancer 1, early onset isoform |
| BRCA2 | NM_000059 | breast cancer 2, early onset |
| BRMS1L | NM_032352 | breast cancer metastasis-suppressor 1-like |
| BRP44L | NM_016098 | brain protein 44-like |
| BRWD1 | NM_001007246 | bromodomain and WD repeat domain |
| | | containing 1 |
| BRWD2 | NM_018117 | bromodomain and WD repeat domain |
| | | containing 2 |
| BTBD11 | NM_001017523 | BTB (POZ) domain containing 11 isoform 2 |
| BTBD15 | NM_014155 | BTB (POZ) domain containing 15 |
| BTBD7 | NM_001002860 | BTB (POZ) domain containing 7 isoform 1 |
| BTN2A1 | NM_007049 | butyrophilin, subfamily 2, member A1 isoform 1 |
| BTRC | NM_003939 | beta-transducin repeat containing protein |
| BVES | NM_007073 | blood vessel epicardial substance |
| C10orf108 | NM_001012714 | hypothetical protein LOC414235 |
| C10orf26 | NM_017787 | hypothetical protein LOC54838 |
| C10orf39 | NM_194303 | hypothetical protein LOC282973 |
| C10orf46 | NM_153810 | hypothetical protein LOC143384 |
| C10orf47 | NM_153256 | hypothetical protein LOC254427 |
| C10orf56 | NM_153367 | hypothetical protein LOC219654 |
| C10orf6 | NM_018121 | hypothetical protein LOC55719 |
| C10orf81 | NM_024889 | hypothetical protein LOC79949 |
| C10orf91 | NM_173541 | hypothetical protein LOC170393 |
| C11orf58 | NM_014267 | small acidic protein |
| C11orf61 | NM_024631 | hypothetical protein LOC79684 |
| C11orf72 | NM_173578 | hypothetical protein LOC283135 |
| C12orf22 | NM_030809 | TGF-beta induced apoptosis protein 12 |
| C12orf34 | NM_032829 | hypothetical protein LOC84915 |
| C12orf4 | NM_020374 | hypothetical protein LOC57102 |
| C12orf41 | NM_017822 | hypothetical protein LOC54934 |
| C12orf47 | NM_016534 | apoptosis-related protein PNAS-1 |
| C12orf49 | NM_024738 | hypothetical protein LOC79794 |
| C12orf51 | NM_173813 | hypothetical protein LOC283450 |
| C12orf59 | NM_153022 | hypothetical protein LOC120939 |
| C13orf10 | NM_022118 | cutaneous T-cell lymphoma tumor antigen |
| | | se70-2 |
| C13orf3 | NM_145061 | hypothetical protein LOC221150 |
| C14orf118 | NM_017926 | hypothetical protein LOC55668 isoform 1 |
| C14orf129 | NM_016472 | hypothetical protein LOC51527 |
| C14orf139 | NM_024633 | hypothetical protein LOC79686 |
| C14orf147 | NM_138288 | hypothetical protein LOC171546 |
| C14orf162 | NM_020181 | chromosome 14 open reading frame 162 |
| C14orf28 | NM_001017923 | hypothetical protein LOC122525 |
| C14orf37 | NM_001001872 | hypothetical protein LOC145407 |
| C14orf58 | NM_017791 | hypothetical protein LOC55640 |
| C14orf92 | NM_014828 | epidermal Langerhans cell protein LCP1 |
| C15orf33 | NM_152647 | hypothetical protein LOC196951 |
| C15orf41 | NM_032499 | hypothetical protein LOC84529 |
| C16orf63 | NM_144600 | hypothetical protein LOC123811 |
| C16orf69 | NM_153261 | hypothetical protein LOC255919 |
| C17orf57 | NM_152347 | hypothetical protein LOC124989 |
| C17orf58 | NM_181656 | hypothetical protein LOC284018 isoform b |
| C17orf71 | NM_018149 | hypothetical protein LOC55181 |
| C17orf75 | NM_022344 | protein kinase Njmu-R1 |
| C18orf1 | NM_001003674 | hypothetical protein LOC753 isoform gamma 1 |
| C18orf16 | NM_153010 | hypothetical protein LOC147429 |
| C18orf19 | NM_152352 | hypothetical protein LOC125228 |
| C18orf4 | NM_032160 | hypothetical protein LOC92126 |
| C1GALT1 | NM_020156 | core 1 synthase, |
| C1orf119 | NM_020141 | hypothetical protein LOC56900 |
| C1orf130 | NM_001010980 | hypothetical protein LOC400746 |
| C1orf140 | NM_001010913 | hypothetical protein LOC400804 |
| C1orf141 | NM_001013674 | hypothetical protein LOC400757 |
| C1orf166 | NM_024544 | hypothetical protein LOC79594 |
| C1orf173 | NM_001002912 | hypothetical protein LOC127254 |
| C1orf24 | NM_052966 | niban protein isoform 2 |
| C1orf25 | NM_030934 | N2,N2-dimethylguanosine tRNA |
| C1orf26 | NM_017673 | hypothetical protein LOC54823 |
| C1orf27 | NM_017847 | odorant response abnormal 4 |
| C1orf63 | NM_207035 | hypothetical protein LOC57035 isoform 1 |
| C1orf69 | NM_001010867 | hypothetical protein LOC200205 |
| C1orf84 | NM_182518 | RP11-506B15.1 protein isoform 3 |
| C1orf86 | NM_182533 | hypothetical protein LOC199990 |
| C1orf96 | NM_145257 | hypothetical protein LOC126731 |
| C20orf12 | NM_018152 | hypothetical protein LOC55184 |
| C20orf133 | NM_001033086 | hypothetical protein LOC140733 isoform 1 |
| C20orf186 | NM_182519 | antimicrobial peptide RY2G5 |
| C20orf29 | NM_018347 | hypothetical protein LOC55317 |
| C20orf54 | NM_033409 | hypothetical protein LOC113278 |
| C21orf58 | NM_199071 | hypothetical protein LOC54058 isoform 2 |
| C21orf91 | NM_017447 | hypothetical protein LOC54149 |
| C2orf26 | NM_023016 | hypothetical protein LOC65124 |
| C2orf3 | NM_003203 | hypothetical protein LOC6936 |
| C2orf37 | NM_025000 | hypothetical protein LOC80067 |
| C3orf17 | NM_001025072 | hypothetical protein LOC25871 isoform b |
| C3orf38 | NM_173824 | hypothetical protein LOC285237 |
| C3orf58 | NM_173552 | hypothetical protein LOC205428 |
| C3orf63 | NM_015224 | retinoblastoma-associated protein 140 |
| C4orf12 | NM_205857 | FBI4 protein |
| C4orf15 | NM_024511 | hypothetical protein LOC79441 |
| C5 | NM_001735 | complement component 5 |
| C5orf14 | NM_024715 | disulfide isomerase |
| C5orf15 | NM_020199 | hypothetical protein LOC56951 |
| C5orf23 | NM_024563 | hypothetical protein LOC79614 |
| C5orf24 | NM_152409 | hypothetical protein LOC134553 |
| C5orf5 | NM_016603 | chromosome 5 open reading frame 5 |
| C6orf117 | NM_138409 | hypothetical protein LOC112609 |
| C6orf120 | NM_001029863 | hypothetical protein LOC387263 |
| C6orf134 | NM_001031722 | hypothetical protein LOC79969 isoform 1 |
| C6orf139 | NM_018132 | hypothetical protein LOC55166 |
| C6orf145 | NM_183373 | hypothetical protein LOC221749 |
| C6orf152 | NM_181714 | hypothetical protein LOC167691 |
| C6orf174 | NM_001012279 | hypothetical protein LOC387104 |
| C6orf199 | NM_145025 | hypothetical protein LOC221264 |
| C6orf47 | NM_021184 | G4 protein |
| C6orf62 | NM_030939 | chromosome 6 open reading frame 62 |
| C6orf71 | NM_203395 | chromosome 6 open reading frame 71 |
| C8orf1 | NM_004337 | hypothetical protein LOC734 |
| C8orf13 | NM_053279 | hypothetical protein LOC83648 |
| C8orf15 | NM_001033662 | hypothetical protein LOC439940 |
| C8orf31 | NM_173687 | hypothetical protein LOC286122 |
| C8orf32 | NM_018024 | hypothetical protein LOC55093 |
| C9orf25 | NM_147202 | hypothetical protein LOC203259 |
| C9orf47 | NM_001001938 | hypothetical protein LOC286223 |
| C9orf48 | NM_194313 | hypothetical protein LOC347240 |
| C9orf5 | NM_032012 | hypothetical protein LOC23731 |
| CA13 | NM_198584 | carbonic anhydrase XIII |
| CA5B | NM_007220 | carbonic anhydrase VB, mitochondrial |
| | | precursor |
| CACHD1 | NM_020925 | cache domain containing 1 |
| CACNA2D4 | NM_001005737 | voltage-gated calcium channel alpha(2)delta-4 |
| CACNB4 | NM_000726 | calcium channel, voltage-dependent, beta 4 |
| CALCR | NM_001742 | calcitonin receptor |
| CALD1 | NM_004342 | caldesmon 1 isoform 2 |
| CALU | NM_001219 | calumenin precursor |
| CAMK2D | NM_172127 | calcium/calmodulin-dependent protein kinase |
| | | II |
| CAMSAP1L1 | NM_203459 | calmodulin regulated spectrin-associated |
| | | protein |
| CARD4 | NM_006092 | caspase recruitment domain family, member 4 |
| CARD8 | NM_014959 | caspase recruitment domain family, member 8 |
| CARF | NM_017632 | collaborates/cooperates with ARF (alternate |
| CASD1 | NM_022900 | CAS1 domain containing 1 |
| CASR | NM_000388 | calcium-sensing receptor |
| CAST | NM_173060 | calpastatin isoform b |
| CBFA2T2 | NM_001032999 | core-binding factor, runt domain, alpha |
| | | subunit |
| CBL | NM_005188 | Cas-Br-M (murine) ecotropic retroviral |
| CBX4 | NM_003655 | chromobox homolog 4 |
| CCDC25 | NM_001031708 | coiled-coil domain containing 25 isoform 1 |
| CCDC3 | NM_031455 | coiled-coil domain containing 3 |
| CCDC34 | NM_080654 | hypothetical protein LOC91057 isoform 2 |
| CCDC4 | NM_207406 | hypothetical protein LOC389206 |
| CCDC43 | NM_144609 | hypothetical protein LOC124808 |
| CCDC82 | NM_024725 | coiled-coil domain containing 82 |
| CCDC93 | NM_019044 | hypothetical protein LOC54520 |
| CCDC98 | NM_139076 | coiled-coil domain containing 98 |
| CCND1 | NM_053056 | cyclin D1 |
| CCNG2 | NM_004354 | cyclin G2 |
| CCNJ | NM_019084 | cyclin J |
| CCR2 | NM_000647 | chemokine (C-C motif) receptor 2 isoform A |
| CCT4 | NM_006430 | chaperonin containing TCP1, subunit 4 (delta) |
| CD160 | NM_007053 | CD160 antigen |
| CD209 | NM_021155 | CD209 antigen |
| CD274 | NM_014143 | CD274 antigen |
| CD58 | NM_001779 | CD58 antigen, (lymphocyte function- |
| | | associated |
| CD59 | NM_000611 | CD59 antigen p18-20 |
| CD80 | NM_005191 | CD80 antigen (CD28 antigen ligand 1, B7-1 |
| CD84 | NM_003874 | CD84 antigen (leukocyte antigen) |
| CD96 | NM_005816 | CD96 antigen isoform 2 precursor |
| CDC25B | NM_004358 | cell division cycle 25B isoform 2 |
| CDC42EP3 | NM_006449 | Cdc42 effector protein 3 |
| CDCA4 | NM_017955 | cell division cycle associated 4 |
| CDCA7 | NM_031942 | cell division cycle associated protein 7 isoform |
| CDCP1 | NM_022842 | CUB domain-containing protein 1 isoform 1 |
| CDH1 | NM_004360 | cadherin 1, type 1 preproprotein |
| CDH17 | NM_004063 | cadherin 17 precursor |
| CDH6 | NM_004932 | cadherin 6, type 2 preproprotein |
| CDK5R1 | NM_003885 | cyclin-dependent kinase 5, regulatory subunit 1 |
| CDKN1A | NM_000389 | cyclin-dependent kinase inhibitor 1A |
| CDKN1B | NM_004064 | cyclin-dependent kinase inhibitor 1B |
| CDR2 | NM_001802 | cerebellar degeneration-related protein 2 |
| CDS2 | NM_003818 | phosphatidate cytidylyltransferase 2 |
| CDYL | NM_004824 | chromodomain protein, Y chromosome-like |
| | | isoform |
| CEBPA | NM_004364 | CCAAT/enhancer binding protein alpha |
| CEBPG | NM_001806 | CCAAT/enhancer binding protein gamma |
| CENTG2 | NM_014914 | centaurin, gamma 2 isoform 2 |
| CEP192 | NM_018069 | hypothetical protein LOC55125 isoform 2 |
| CEP350 | NM_014810 | centrosome-associated protein 350 |
| CEP55 | NM_018131 | centrosomal protein 55 kDa |
| CEP70 | NM_024491 | centrosomal protein 70 kDa |
| CFH | NM_000186 | complement factor H isoform a precursor |
| CFHR1 | NM_002113 | complement factor H-related 1 |
| CFHR5 | NM_030787 | complement factor H-related 5 |
| CFL2 | NM_021914 | cofilin 2 |
| CFTR | NM_000492 | cystic fibrosis transmembrane conductance |
| CGGBP1 | NM_001008390 | CGG triplet repeat binding protein 1 |
| CHAC2 | NM_001008708 | hypothetical protein LOC494143 |
| CHCHD3 | NM_017812 | coiled-coil-helix-coiled-coil-helix domain |
| CHCHD8 | NM_016565 | coiled-coil-helix-coiled-coil-helix domain |
| CHD1 | NM_001270 | chromodomain helicase DNA binding protein 1 |
| CHD6 | NM_032221 | chromodomain helicase DNA binding protein 6 |
| CHD7 | NM_017780 | chromodomain helicase DNA binding protein 9 |
| CHD9 | NM_025134 | chromodomain helicase DNA binding protein 9 |
| CHES1 | NM_005197 | checkpoint suppressor 1 |
| CHKB | NM_152253 | choline/ethanolamine kinase isoform b |
| CHML | NM_001821 | choroideremia-like Rab escort protein 2 |
| CHMP2B | NM_014043 | chromatin modifying protein 2B |
| CHMP5 | NM_016410 | chromatin modifying protein 5 |
| CHN2 | NM_004067 | beta chimerin isoform 2 |
| CHORDC1 | NM_012124 | cysteine and histidine-rich domain |
| CHRM2 | NM_000739 | cholinergic receptor, muscarinic 2 |
| CHST3 | NM_004273 | carbohydrate (chondroitin 6) sulfotransferase 3 |
| CHST7 | NM_019886 | carbohydrate (N-acetylglucosamine 6-O) |
| CHSY1 | NM_014918 | carbohydrate (chondroitin) synthase 1 |
| CHURC1 | NM_145165 | churchill domain containing 1 |
| CIT | NM_007174 | citron |
| CLASP1 | NM_015282 | CLIP-associating protein 1 |
| CLASP2 | NM_015097 | CLIP-associating protein 2 |
| CLCF1 | NM_013246 | cardiotrophin-like cytokine factor 1 |
| CLCN6 | NM_001286 | chloride channel 6 isoform ClC-6a |
| CLDND1 | NM_019895 | claudin domain containing 1 protein isoform a |
| CLEC4E | NM_014358 | C-type lectin domain family 4, member E |
| CLEC5A | NM_013252 | C-type lectin, superfamily member 5 |
| CLEC7A | NM_022570 | dendritic cell-associated C-type lectin 1 |
| CLIC4 | NM_013943 | chloride intracellular channel 4 |
| CLLU1 | NM_001025233 | hypothetical protein LOC574028 |
| CLOCK | NM_004898 | clock |
| CLSPN | NM_022111 | claspin |
| CMIP | NM_030629 | c-Maf-inducing protein Tc-mip isoform |
| CNGA2 | NM_005140 | cyclic nucleotide gated channel alpha 2 |
| CNKSR3 | NM_173515 | CNKSR family member 3 |
| CNN3 | NM_001839 | calponin 3 |
| CNOT4 | NM_013316 | CCR4-NOT transcription complex, subunit 4 |
| CNOT6 | NM_015455 | CCR4-NOT transcription complex, subunit 6 |
| CNOT7 | NM_013354 | CCR4-NOT transcription complex, subunit 7 |
| CNOT8 | NM_004779 | CCR4-NOT transcription complex, subunit 8 |
| CNR1 | NM_016083 | central cannabinoid receptor isoform a |
| CNTD1 | NM_173478 | hypothetical protein LOC124817 |
| CNTFR | NM_001842 | ciliary neurotrophic factor receptor |
| CNTNAP2 | NM_014141 | cell recognition molecule Caspr2 precursor |
| COG6 | NM_020751 | component of oligomeric golgi complex 6 |
| COL21A1 | NM_030820 | collagen, type XXI, alpha 1 precursor |
| COL4A3 | NM_000091 | alpha 3 type IV collagen isoform 1 precursor |
| COL9A2 | NM_001852 | alpha 2 type IX collagen |
| COMMD6 | NM_203497 | COMM domain containing 6 isoform a |
| COPA | NM_004371 | coatomer protein complex, subunit alpha |
| COPS8 | NM_006710 | COP9 signalosome subunit 8 isoform 1 |
| COQ5 | NM_032314 | hypothetical protein LOC84274 |
| CORO1C | NM_014325 | coronin, actin binding protein, 1C |
| CORO6 | NM_032854 | coronin 6 |
| COVA1 | NM_006375 | cytosolic ovarian carcinoma antigen 1 isoform a |
| COX11 | NM_004375 | COX11 homolog |
| CPSF2 | NM_017437 | cleavage and polyadenylation specific factor 2 |
| CPSF4 | NM_006693 | cleavage and polyadenylation specific factor 4, |
| CPSF6 | NM_007007 | cleavage and polyadenylation specific factor 6, |
| CPXCR1 | NM_033048 | hypothetical protein LOC53336 |
| CREB5 | NM_001011666 | cAMP responsive element binding protein 5 |
| CREBBP | NM_004380 | CREB binding protein |
| CREBL2 | NM_001310 | cAMP responsive element binding protein-like 2 |
| CREG1 | NM_003851 | cellular repressor of E1A-stimulated genes |
| CRHBP | NM_001882 | corticotropin releasing hormone binding |
| | | protein |
| CRIP2 | NM_001312 | cysteine-rich protein 2 |
| CRKL | NM_005207 | v-crk sarcoma virus CT10 oncogene homolog |
| CROP | NM_006107 | cisplatin resistance-associated overexpressed |
| CROT | NM_021151 | carnitine O-octanoyltransferase |
| CRSP2 | NM_004229 | cofactor required for Sp1 transcriptional |
| CRTAP | NM_006371 | cartilage associated protein precursor |
| CRYZL1 | NM_145858 | crystallin, zeta-like 1 |
| CSF1 | NM_172212 | colony stimulating factor 1 isoform a precursor |
| CSMD3 | NM_052900 | CUB and Sushi multiple domains 3 isoform 3 |
| CSNK1G3 | NM_001031812 | casein kinase 1, gamma 3 isoform 2 |
| CSS3 | NM_175856 | chondroitin sulfate synthase 3 |
| CSTF3 | NM_001033506 | cleavage stimulation factor subunit 3 isoform 3 |
| CTAGE5 | NM_005930 | CTAGE family, member 5 isoform 1 |
| CTCFL | NM_080618 | CCCTC-binding factor-like protein |
| CTDSPL | NM_001008392 | small CTD phosphatase 3 isoform 1 |
| CTNND1 | NM_001331 | catenin (cadherin-associated protein), delta 1 |
| CTNND2 | NM_001332 | catenin (cadherin-associated protein), delta 2 |
| CTNS | NM_004937 | cystinosis, nephropathic isoform 2 |
| CTSB | NM_001908 | cathepsin B preproprotein |
| CTSC | NM_001814 | cathepsin C isoform a preproprotein |
| CTSO | NM_001334 | cathepsin O preproprotein |
| CUGBP2 | NM_001025076 | CUG triplet repeat, RNA binding protein 2 |
| CUL5 | NM_003478 | Vasopressin-activated calcium-mobilizing |
| CUTC | NM_015960 | cutC copper transporter homolog |
| CXorf41 | NM_173494 | hypothetical protein LOC139212 |
| CXorf6 | NM_005491 | hypothetical protein LOC10046 |
| CXX1 | NM_003928 | CAAX box 1 |
| CXXC6 | NM_030625 | CXXC finger 6 |
| CYBB | NM_000397 | cytochrome b-245, beta polypeptide (chronic |
| CYLN2 | NM_003388 | cytoplasmic linker 2 isoform 1 |
| CYP19A1 | NM_000103 | cytochrome P450, family 19 |
| CYP1B1 | NM_000104 | cytochrome P450, family 1, subfamily B, |
| CYP2C9 | NM_000771 | cytochrome P450, family 2, subfamily C, |
| CYP3A43 | NM_022820 | cytochrome P450, family 3, subfamily A, |
| CYP4F2 | NM_001082 | cytochrome P450, family 4, subfamily F, |
| CYP4F3 | NM_000896 | cytochrome P450, family 4, subfamily F, |
| CYP4V2 | NM_207352 | cytochrome P450, family 4, subfamily v, |
| CYYR1 | NM_052954 | cysteine and tyrosine-rich 1 protein precursor |
| DAB2 | NM_001343 | disabled homolog 2 |
| DAB2IP | NM_032552 | DAB2 interacting protein isoform 1 |
| DACH1 | NM_004392 | dachshund homolog 1 isoform c |
| DAG1 | NM_004393 | dystroglycan 1 precursor |
| DAZ1 | NM_004081 | deleted in azoospermia |
| DAZ2 | NM_001005785 | deleted in azoospermia 2 isoform 2 |
| DAZ3 | NM_020364 | deleted in azoospermia 3 |
| DAZ4 | NM_001005375 | deleted in azoospermia 4 isoform 1 |
| DAZL | NM_001351 | deleted in azoospermia-like |
| DBNDD2 | NM_033542 | SCF apoptosis response protein 1 isoform 2 |
| DBR1 | NM_016216 | debranching enzyme homolog 1 |
| DCBLD2 | NM_080927 | discoidin, CUB and LCCL domain containing 2 |
| DCLRE1B | NM_022836 | DNA cross-link repair 1B (PSO2 homolog, S. |
| DCP2 | NM_152624 | DCP2 decapping enzyme |
| DCUN1D1 | NM_020640 | RP42 homolog |
| DCUN1D4 | NM_015115 | DCN1, defective in cullin neddylation 1, |
| | | domain |
| DCX | NM_000555 | doublecortin isoform a |
| DDAH1 | NM_012137 | dimethylarginine dimethylaminohydrolase 1 |
| DDEF1 | NM_018482 | development and differentiation enhancing |
| | | factor |
| DDI1 | NM_001001711 | hypothetical protein LOC414301 |
| DDIT4L | NM_145244 | DNA-damage-inducible transcript 4-like |
| DDX1 | NM_004939 | DEAD (Asp-Glu-Ala-Asp) box polypeptide 1 |
| DDX26B | NM_182540 | hypothetical protein LOC203522 |
| DDX3X | NM_001356 | DEAD/H (Asp-Glu-Ala-Asp/His) box |
| | | polypeptide 3 |
| DDX3Y | NM_004660 | DEAD (Asp-Glu-Ala-Asp) box polypeptide 3, |
| DDX43 | NM_018665 | DEAD (Asp-Glu-Ala-Asp) box polypeptide 43 |
| DDX46 | NM_014829 | DEAD (Asp-Glu-Ala-Asp) box polypeptide 46 |
| DDX53 | NM_182699 | DEAD (Asp-Glu-Ala-Asp) box polypeptide 53 |
| DDX59 | NM_031306 | DEAD (Asp-Glu-Ala-Asp) box polypeptide 59 |
| DEK | NM_003472 | DEK oncogene (DNA binding) |
| DENND2C | NM_198459 | DENN/MADD domain containing 2C |
| DENND4C | NM_017925 | hypothetical protein LOC55667 |
| DERL1 | NM_024295 | Der1-like domain family, member 1 |
| DGKA | NM_001345 | diacylglycerol kinase, alpha 80 kDa |
| DGKE | NM_003647 | diacylglycerol kinase epsilon |
| DIRAS2 | NM_017594 | Di-Ras2 |
| DISC1 | NM_001012957 | disrupted in schizophrenia 1 isoform Lv |
| DIXDC1 | NM_033425 | DIX domain containing 1 isoform b |
| DKFZp434I1020 | NM_194295 | hypothetical protein LOC196968 |
| DKFZp666G057 | NM_001008226 | hypothetical protein LOC283726 |
| DKFZP686A101247 | NM_014988 | hypothetical protein LOC22998 |
| DKFZP686A10121 | NM_033107 | claudin 12 |
| DKFZp686I15217 | NM_207495 | hypothetical protein LOC401232 |
| DKFZp686O24166 | NM_001009913 | hypothetical protein LOC374383 |
| DLC1 | NM_006094 | deleted in liver cancer 1 isoform 2 |
| DLGAP2 | NM_004745 | discs large-associated protein 2 |
| DMN | NM_015286 | desmuslin isoform B |
| DMRT2 | NM_006557 | doublesex and mab-3 related transcription |
| | | factor |
| DMRTB1 | NM_033067 | DMRT-like family B with proline-rich C- |
| | | terminal, |
| DMXL2 | NM_015263 | Dmx-like 2 |
| DNAJA5 | NM_001012339 | DnaJ homology subfamily A member 5 |
| | | isoform 2 |
| DNAJB12 | NM_001002762 | DnaJ (Hsp40) homolog, subfamily B, member |
| | | 12 |
| DNAJB6 | NM_005494 | DnaJ (Hsp40) homolog, subfamily B, member 6 |
| DNAJB9 | NM_012328 | DnaJ (Hsp40) homolog, subfamily B, member 9 |
| DNAJC15 | NM_013238 | DNAJ domain-containing |
| DNAJC5 | NM_025219 | DnaJ (Hsp40) homolog, subfamily C, member 8 |
| DNAJC8 | NM_014280 | DnaJ (Hsp40) homolog, subfamily C, member 8 |
| DNAPTP6 | NM_015535 | hypothetical protein LOC26010 |
| DNM3 | NM_015569 | dynamin 3 |
| DNMT3A | NM_175630 | DNA cytosine methyltransferase 3 alpha |
| | | isoform |
| DOC1 | NM_014890 | downregulated in ovarian cancer 1 isoform 2 |
| DOK5 | NM_018431 | DOK5 protein isoform a |
| DP58 | NM_001004441 | cytosolic phosphoprotein DP58 |
| DPCR1 | NM_080870 | diffuse panbronchiolitis critical region 1 |
| DPP10 | NM_001004360 | dipeptidyl peptidase 10 isoform short |
| DR1 | NM_001938 | down-regulator of transcription 1 |
| DRP2 | NM_001939 | dystrophin related protein 2 |
| DSC3 | NM_001941 | desmocollin 3 isoform Dsc3a preproprotein |
| DSG4 | NM_177986 | desmoglein 4 |
| DTNA | NM_001390 | dystrobrevin alpha isoform 1 |
| DUOX2 | NM_014080 | dual oxidase 2 precursor |
| DUS4L | NM_181581 | dihydrouridine synthase 4-like |
| DUSP1 | NM_004417 | dual specificity phosphatase 1 |
| DYNLRB2 | NM_130897 | dynein, cytoplasmic, light polypeptide 2B |
| DZIP1 | NM_014934 | DAZ interacting protein 1 isoform 1 |
| E2F3 | NM_001949 | E2F transcription factor 3 |
| EDA | NM_001005609 | ectodysplasin A isoform EDA-A2 |
| EDEM3 | NM_025191 | ER degradation enhancer, mannosidase alpha- |
| | | like |
| EDG3 | NM_005226 | endothelial differentiation, sphingolipid |
| EDNRA | NM_001957 | endothelin receptor type A |
| EDNRB | NM_000115 | endothelin receptor type B isoform 1 |
| EED | NM_152991 | embryonic ectoderm development isoform b |
| EEF2K | NM_013302 | elongation factor-2 kinase |
| EFCAB5 | NM_001033562 | EF-hand calcium binding domain 5 isoform 2 |
| EFCBP1 | NM_022351 | EF hand calcium binding protein 1 |
| EFNA1 | NM_004428 | ephrin A1 isoform a precursor |
| EGR1 | NM_001964 | early growth response 1 |
| EGR3 | NM_004430 | early growth response 3 |
| EHD1 | NM_006795 | EH-domain containing 1 |
| EHD3 | NM_014600 | EH-domain containing 3 |
| EIF1AX | NM_001412 | X-linked eukaryotic translation initiation |
| EIF4B | NM_001417 | eukaryotic translation initiation factor 4B |
| EIF5 | NM_001969 | eukaryotic translation initiation factor 5 |
| EIF5A2 | NM_020390 | eIF-5A2 protein |
| EIF5B | NM_015904 | eukaryotic translation initiation factor 5B |
| ELAC1 | NM_018696 | elaC homolog 1 |
| ELAVL4 | NM_021952 | ELAV-like 4 |
| ELF2 | NM_006874 | E74-like factor 2 (ets domain transcription |
| ELL | NM_006532 | elongation factor RNA polymerase II |
| ELMO2 | NM_133171 | engulfment and cell motility 2 |
| ELMOD1 | NM_018712 | ELMO domain containing 1 |
| ELOVL6 | NM_024090 | ELOVL family member 6, elongation of long |
| | | chain |
| ENAH | NM_001008493 | enabled homolog isoform a |
| ENDOG | NM_004435 | endonuclease G precursor |
| ENSA | NM_004436 | endosulfine alpha isoform 3 |
| ENTH | NM_014666 | enthoprotin |
| ENTPD3 | NM_001248 | ectonucleoside triphosphate |
| | | diphosphohydrolase |
| ENTPD5 | NM_001249 | ectonucleoside triphosphate |
| | | diphosphohydrolase |
| EPB41L4B | NM_019114 | erythrocyte membrane protein band 4.1 like |
| | | 4B |
| EPDR1 | NM_017549 | upregulated in colorectal cancer gene 1 protein |
| EPHA3 | NM_182644 | ephrin receptor EphA3 isoform b precursor |
| EPHA4 | NM_004438 | ephrin receptor EphA4 |
| EPM2AIP1 | NM_014805 | EPM2A interacting protein 1 |
| EPN2 | NM_014964 | epsin 2 isoform b |
| EPOR | NM_000121 | erythropoietin receptor precursor |
| EPS8 | NM_004447 | epidermal growth factor receptor pathway |
| EPS8L2 | NM_022772 | epidermal growth factor receptor pathway |
| ERBB2IP | NM_001006600 | ERBB2 interacting protein isoform 7 |
| ERCC8 | NM_000082 | excision repair cross-complementing rodent |
| EREG | NM_001432 | epiregulin precursor |
| ERG | NM_004449 | v-ets erythroblastosis virus E26 oncogene like |
| ERGIC1 | NM_020462 | endoplasmic reticulum-golgi intermediate |
| ERRFI1 | NM_018948 | mitogen-inducible gene 6 protein |
| ESCO2 | NM_001017420 | establishment of cohesion 1 homolog 2 |
| ESRRG | NM_001438 | estrogen-related receptor gamma isoform 1 |
| ETNK1 | NM_018638 | ethanolamine kinase 1 isoform A |
| ETS2 | NM_005239 | v-ets erythroblastosis virus E26 oncogene |
| ETV1 | NM_004956 | ets variant gene 1 |
| ETV5 | NM_004454 | ets variant gene 5 (ets-related molecule) |
| ETV6 | NM_001987 | ets variant gene 6 |
| EVI2B | NM_006495 | ecotropic viral integration site 2B |
| EVI5 | NM_005665 | ecotropic viral integration site 5 |
| EXOC2 | NM_018303 | Sec5 protein |
| EXOC5 | NM_006544 | SEC10 protein |
| EXOC6 | NM_001013848 | SEC15-like 1 isoform b |
| EXOSC3 | NM_001002269 | exosome component 3 isoform 2 |
| EXOSC6 | NM_058219 | homolog of yeast mRNA transport regulator 3 |
| EYA2 | NM_005244 | eyes absent 2 isoform a |
| F3 | NM_001993 | coagulation factor III precursor |
| FADS1 | NM_013402 | fatty acid desaturase 1 |
| FALZ | NM_004459 | fetal Alzheimer antigen isoform 2 |
| FAM102B | NM_001010883 | hypothetical protein LOC284611 |
| FAM107B | NM_031453 | hypothetical protein LOC83641 |
| FAM116A | NM_152678 | hypothetical protein LOC201627 |
| FAM13C1 | NM_001001971 | hypothetical protein LOC220965 isoform 2 |
| FAM20B | NM_014864 | family with sequence similarity 20, member B |
| FAM21C | NM_015262 | hypothetical protein LOC253725 |
| FAM26C | NM_001001412 | hypothetical protein LOC255022 |
| FAM38B | NM_022068 | hypothetical protein LOC63895 |
| FAM3B | NM_058186 | family with sequence similarity 3, member B |
| FAM44A | NM_148894 | family with sequence similarity 44, member A |
| FAM46D | NM_152630 | hypothetical protein LOC169966 |
| FAM60A | NM_021238 | family with sequence similarity 60, member A |
| FAM62B | NM_020728 | family with sequence similarity 62 (C2 domain |
| FAM73A | NM_198549 | hypothetical protein LOC374986 |
| FAM76B | NM_144664 | hypothetical protein LOC143684 |
| FAM81A | NM_152450 | hypothetical protein LOC145773 |
| FAM83D | NM_030919 | hypothetical protein LOC81610 |
| FAM8A1 | NM_016255 | Autosomal Highly Conserved Protein |
| FARP1 | NM_005766 | FERM, RhoGEF, and pleckstrin domain |
| | | protein 1 |
| FAS | NM_000043 | tumor necrosis factor receptor superfamily, |
| FASLG | NM_000639 | fas ligand |
| FAT2 | NM_001447 | FAT tumor suppressor 2 precursor |
| FBLN5 | NM_006329 | fibulin 5 precursor |
| FBN2 | NM_001999 | fibrillin 2 precursor |
| FBXL16 | NM_153350 | F-box and leucine-rich repeat protein 16 |
| FBXO21 | NM_015002 | F-box only protein 21 isoform 2 |
| FBXO22 | NM_147188 | F-box only protein 22 isoform a |
| FBXO4 | NM_033484 | F-box only protein 4 isoform 2 |
| FBXW11 | NM_012300 | F-box and WD-40 domain protein 1B isoform C |
| FBXW2 | NM_012164 | F-box and WD-40 domain protein 2 |
| FBXW7 | NM_001013415 | F-box protein FBW7 isoform 3 |
| FCMD | NM_006731 | fukutin |
| FCRL4 | NM_031282 | Fc receptor-like 4 |
| FECH | NM_000140 | ferrochelatase isoform b precursor |
| FER1L3 | NM_013451 | myoferlin isoform a |
| FEZ2 | NM_005102 | zygin 2 |
| FGD1 | NM_004463 | faciogenital dysplasia protein |
| FGF2 | NM_002006 | fibroblast growth factor 2 |
| FGF23 | NM_020638 | fibroblast growth factor 23 precursor |
| FGF5 | NM_004464 | fibroblast growth factor 5 isoform 1 precursor |
| FGFR2 | NM_023028 | fibroblast growth factor receptor 2 isoform 10 |
| FHL1 | NM_001449 | four and a half LIM domains 1 |
| FHOD1 | NM_013241 | formin homology 2 domain containing 1 |
| FIGN | NM_018086 | fidgetin |
| FIGNL1 | NM_022116 | fidgetin-like 1 |
| FKBP1A | NM_000801 | FK506-binding protein 1A |
| FKBP9 | NM_007270 | FK506 binding protein 9 |
| FKBP9L | NM_182827 | FK506 binding protein 9-like |
| FKSG44 | NM_031904 | FKSG44 protein |
| FLG | NM_002016 | filaggrin |
| FLI1 | NM_002017 | Friend leukemia virus integration 1 |
| FLJ10241 | NM_018035 | hypothetical protein LOC55101 |
| FLJ10292 | NM_018048 | mago-nashi homolog |
| FLJ10357 | NM_018071 | hypothetical protein LOC55701 |
| FLJ10781 | NM_018215 | hypothetical protein LOC55228 |
| FLJ10803 | NM_018224 | hypothetical protein LOC55744 |
| FLJ10815 | NM_018231 | amino acid transporter |
| FLJ10925 | NM_018275 | hypothetical protein LOC55262 |
| FLJ11021 | NM_023012 | hypothetical protein LOC65117 isoform a |
| FLJ11171 | NM_018348 | hypothetical protein LOC55783 |
| FLJ11184 | NM_018352 | hypothetical protein LOC55319 |
| FLJ12505 | NM_024749 | hypothetical protein LOC79805 |
| FLJ13197 | NM_024614 | hypothetical protein LOC79667 |
| FLJ16323 | NM_001004352 | hypothetical protein LOC441390 |
| FLJ16542 | NM_001004301 | hypothetical protein LOC126017 |
| FLJ20032 | NM_017628 | hypothetical protein LOC54790 |
| FLJ20035 | NM_017631 | hypothetical protein LOC55601 |
| FLJ20232 | NM_019008 | hypothetical protein LOC54471 |
| FLJ20294 | NM_017749 | hypothetical protein LOC55626 |
| FLJ20298 | NM_017752 | hypothetical protein LOC54885 isoform a |
| FLJ20558 | NM_017880 | hypothetical protein LOC54980 |
| FLJ20859 | NM_001029991 | FLJ20859 protein isoform 1 |
| FLJ21986 | NM_024913 | hypothetical protein LOC79974 |
| FLJ25476 | NM_152493 | hypothetical protein LOC149076 |
| FLJ25680 | NM_153216 | hypothetical protein LOC134187 |
| FLJ30046 | NM_144595 | hypothetical protein LOC122060 B |
| FLJ30313 | NM_152757 | hypothetical protein LOC253868 |
| FLJ30596 | NM_153013 | hypothetical protein LOC133686 |
| FLJ30851 | NM_198553 | hypothetical protein LOC375190 |
| FLJ31659 | NM_153027 | hypothetical protein LOC152756 |
| FLJ31818 | NM_152556 | hypothetical protein LOC154743 |
| FLJ31846 | NM_144974 | hypothetical protein LOC160857 |
| FLJ32028 | NM_152680 | hypothetical protein LOC201799 |
| FLJ33814 | NM_173510 | hypothetical protein LOC150275 |
| FLJ35630 | NM_152618 | hypothetical protein LOC166379 |
| FLJ36004 | NM_152590 | hypothetical protein FLJ36004 |
| FLJ36180 | NM_178556 | hypothetical protein LOC339976 |
| FLJ36492 | NM_182568 | hypothetical protein LOC284047 |
| FLJ37538 | NM_173564 | hypothetical protein FLJ37538 |
| FLJ37543 | NM_173667 | hypothetical protein LOC285668 |
| FLJ38288 | NM_173632 | hypothetical protein LOC284309 |
| FLJ39531 | NM_207445 | hypothetical protein LOC400360 |
| FLJ40298 | NM_173486 | hypothetical protein LOC129852 |
| FLJ40432 | NM_152523 | hypothetical protein LOC151195 |
| FLJ40919 | NM_182508 | hypothetical protein LOC144809 |
| FLJ41131 | NM_198476 | hypothetical protein LOC284325 |
| FLJ44006 | NM_001001696 | hypothetical protein LOC400997 |
| FLJ44313 | NM_207460 | hypothetical protein LOC400658 |
| FLJ45139 | NM_001001692 | hypothetical protein LOC400867 |
| FLJ45248 | NM_207505 | hypothetical protein LOC401472 |
| FLJ45337 | NM_207465 | hypothetical protein LOC400754 |
| FLJ45422 | NM_001004349 | hypothetical protein LOC441140 |
| FLJ45537 | NM_001001709 | hypothetical protein LOC401535 |
| FLJ45974 | NM_001001707 | hypothetical protein LOC401337 |
| FLJ46082 | NM_207417 | hypothetical protein LOC389799 |
| FLJ90757 | NM_001004336 | hypothetical protein LOC440465 |
| FLRT2 | NM_013231 | fibronectin leucine rich transmembrane protein |
| FLT1 | NM_002019 | fms-related tyrosine kinase 1 (vascular |
| FLT4 | NM_002020 | fms-related tyrosine kinase 4 isoform 2 |
| FMNL2 | NM_052905 | formin-like 2 |
| FMO2 | NM_001460 | flavin containing monooxygenase 2 |
| FN1 | NM_002026 | fibronectin 1 isoform 3 preproprotein |
| FNDC1 | NM_032532 | fibronectin type III domain containing 1 |
| FNDC3B | NM_022763 | fibronectin type III domain containing 3B |
| FNTB | NM_002028 | farnesyltransferase, CAAX box, beta |
| FOXD4 | NM_207305 | forkhead box D4 |
| FOXD4L2 | NM_199135 | FOXD4-like 2 |
| FOXF1 | NM_001451 | forkhead box F1 |
| FOXG1B | NM_005249 | forkhead box G1B |
| FOXL2 | NM_023067 | forkhead box L2 |
| FOXO1A | NM_002015 | forkhead box O1A |
| FOXP1 | NM_032682 | forkhead box P1 isoform 1 |
| FRAS1 | NM_025074 | Fraser syndrome 1 isoform 1 |
| FREM1 | NM_144966 | FRAS1 related extracellular matrix 1 |
| FRMD4A | NM_018027 | FERM domain containing 4A |
| FRMD6 | NM_152330 | FERM domain containing 6 |
| FSD1L | NM_207647 | fibronectin type III and SPRY domain |
| | | containing |
| FSIP1 | NM_152597 | fibrous sheath interacting protein 1 |
| FSTL1 | NM_007085 | follistatin-like 1 precursor |
| FUBP1 | NM_003902 | far upstream element-binding protein |
| FUNDC1 | NM_173794 | FUN14 domain containing 1 |
| FUSIP1 | NM_006625 | FUS interacting protein (serine-arginine rich) 1 |
| FUT10 | NM_032664 | fucosyltransferase 10 |
| FUT4 | NM_002033 | fucosyltransferase 4 |
| FVT1 | NM_002035 | follicular lymphoma variant translocation 1 |
| FYTTD1 | NM_001011537 | forty-two-three domain containing 1 isoform 2 |
| FZD1 | NM_003505 | frizzled 1 |
| FZD3 | NM_017412 | frizzled 3 |
| FZD4 | NM_012193 | frizzled 4 |
| G6PC | NM_000151 | glucose-6-phosphatase, catalytic |
| GAA | NM_000152 | acid alpha-glucosidase preproprotein |
| GAB1 | NM_002039 | GRB2-associated binding protein 1 isoform b |
| GABARAPL1 | NM_031412 | GABA(A) receptor-associated protein like 1 |
| GABARAPL2 | NM_007285 | GABA(A) receptor-associated protein-like 2 |
| GABPA | NM_002040 | GA binding protein transcription factor, alpha |
| GABPB2 | NM_002041 | GA binding protein transcription factor, beta |
| GABRA4 | NM_000809 | gamma-aminobutyric acid A receptor, alpha 4 |
| GABRB3 | NM_000814 | gamma-aminobutyric acid (GABA) A |
| | | receptor, beta |
| GADL1 | NM_207359 | glutamate decarboxylase-like 1 |
| GALNAC4S-6ST | NM_015892 | B cell RAG associated protein |
| GALNT10 | NM_017540 | GalNAc transferase 10 isoform b |
| GALNT2 | NM_004481 | polypeptide N-acetylgalactosaminyltransferase 2 |
| GART | NM_175085 | phosphoribosylglycinamide formyltransferase, |
| GAS2 | NM_005256 | growth arrest-specific 2 |
| GAS7 | NM_003644 | growth arrest-specific 7 isoform a |
| GATA2 | NM_032638 | GATA binding protein 2 |
| GATAD2B | NM_020699 | GATA zinc finger domain containing 2B |
| GCNT2 | NM_001491 | glucosaminyl (N-acetyl) transferase 2, |
| Gcom1 | NM_001018100 | GRINL1A upstream protein isoform 7 |
| GDF6 | NM_001001557 | growth differentiation factor 6 |
| GDI2 | NM_001494 | GDP dissociation inhibitor 2 |
| Gene_symbol | hsa-miR-200c | Gene_name |
| | target | |
| GFAP | NM_002055 | glial fibrillary acidic protein |
| GLDN | NM_181789 | collomin |
| GLE1L | NM_001003722 | GLE1-like, RNA export mediator isoform 1 |
| GLI3 | NM_000168 | GLI-Kruppel family member GLI3 |
| GLRA2 | NM_002063 | glycine receptor, alpha 2 |
| GLRX | NM_002064 | glutaredoxin (thioltransferase) |
| GM2A | NM_000405 | GM2 ganglioside activator precursor |
| GMFB | NM_004124 | glia maturation factor, beta |
| GNA13 | NM_006572 | guanine nucleotide binding protein (G |
| | | protein), |
| GNAI3 | NM_006496 | guanine nucleotide binding protein (G |
| | | protein), |
| GNAT1 | NM_144499 | guanine nucleotide binding protein, alpha |
| GNG12 | NM_018841 | G-protein gamma-12 subunit |
| GOLGA1 | NM_002077 | golgin 97 |
| GOLGA7 | NM_001002296 | golgi autoantigen, golgin subfamily a, 7 |
| GOLGA8E | NM_001012423 | golgi autoantigen, golgin family member |
| GOLGA8G | NM_001012420 | hypothetical protein LOC283768 |
| GOLPH4 | NM_014498 | golgi phosphoprotein 4 |
| GOLT1B | NM_016072 | golgi transport 1 homolog B |
| GORASP2 | NM_015530 | golgi reassembly stacking protein 2 |
| GOSR2 | NM_004287 | golgi SNAP receptor complex member 2 |
| | | isoform A |
| GOT1 | NM_002079 | aspartate aminotransferase 1 |
| GPIAP1 | NM_005898 | membrane component chromosome 11 surface |
| | | marker |
| GPM6A | NM_005277 | glycoprotein M6A isoform 1 |
| GPR116 | NM_015234 | G-protein coupled receptor 116 |
| GPR180 | NM_180989 | G protein-coupled receptor 180 precursor |
| GPR62 | NM_080865 | G protein-coupled receptor 62 |
| GPR84 | NM_020370 | inflammation-related G protein-coupled |
| | | receptor |
| GPR85 | NM_018970 | G protein-coupled receptor 85 |
| GPR92 | NM_020400 | putative G protein-coupled receptor 92 |
| GPRASP2 | NM_001004051 | G protein-coupled receptor associated sorting |
| GRB10 | NM_001001549 | growth factor receptor-bound protein 10 |
| | | isoform |
| GREB1 | NM_014668 | GREB1 protein isoform a |
| GREM1 | NM_013372 | gremlin-1 precursor |
| GREM2 | NM_022469 | gremlin 2 precursor |
| GRM5 | NM_000842 | glutamate receptor, metabotropic 5 precursor |
| GSTA4 | NM_001512 | glutathione S-transferase A4 |
| GSTM3 | NM_000849 | glutathione S-transferase M3 |
| GTF2E1 | NM_005513 | general transcription factor IIE, polypeptide 1 |
| GTF3C2 | NM_001521 | general transcription factor IIIC, polypeptide |
| GUCY1A3 | NM_000856 | guanylate cyclase 1, soluble, alpha 3 |
| GYS2 | NM_021957 | glycogen synthase 2 (liver) |
| H2AFJ | NM_018267 | H2A histone family, member J isoform 1 |
| HAL | NM_002108 | histidine ammonia-lyase |
| HAS2 | NM_005328 | hyaluronan synthase 2 |
| HBS1L | NM_006620 | HBS1-like |
| HCCS | NM_005333 | holocytochrome c synthase (cytochrome c |
| HCFC2 | NM_013320 | host cell factor C2 |
| HDAC4 | NM_006037 | histone deacetylase 4 |
| HECTD2 | NM_182765 | HECT domain containing 2 isoform a |
| HEMK1 | NM_016173 | HemK methyltransferase family member 1 |
| HERC3 | NM_014606 | hect domain and RLD 3 |
| HERC4 | NM_001017972 | hect domain and RLD 4 isoform c |
| HFE | NM_000410 | hemochromatosis protein isoform 1 precursor |
| HGD | NM_000187 | homogentisate 1,2-dioxygenase |
| HIC2 | NM_015094 | hypermethylated in cancer 2 |
| HISPPD1 | NM_015216 | Histidine acid phosphatase domain containing 1 |
| HK2 | NM_000189 | hexokinase 2 |
| HLA-DOA | NM_002119 | major histocompatibility complex, class II, DO |
| HLF | NM_002126 | hepatic leukemia factor |
| HM13 | NM_178582 | minor histocompatibility antigen 13 isoform 4 |
| HMBOX1 | NM_024567 | hypothetical protein LOC79618 |
| HMGB1 | NM_002128 | high-mobility group box 1 |
| HMGCLL1 | NM_019036 | 3-hydroxymethyl-3-methylglutaryl-Coenzyme A |
| HMOX1 | NM_002133 | heme oxygenase (decyclizing) 1 |
| HNRNPG-T | NM_014469 | testes-specific heterogenous nuclear |
| HNRPD | NM_001003810 | heterogeneous nuclear ribonucleoprotein D |
| HNRPH1 | NM_005520 | heterogeneous nuclear ribonucleoprotein H1 |
| HNRPU | NM_004501 | heterogeneous nuclear ribonucleoprotein U |
| HOXA1 | NM_005522 | homeobox A1 isoform a |
| HOXA5 | NM_019102 | homeobox A5 |
| HPS5 | NM_007216 | Hermansky-Pudlak syndrome 5 isoform b |
| HPSE | NM_006665 | heparanase |
| HRB | NM_004504 | HIV-1 Rev binding protein |
| HRB2 | NM_007043 | HIV-1 rev binding protein 2 |
| HS2ST1 | NM_012262 | heparan sulfate 2-O-sulfotransferase 1 |
| HS3ST1 | NM_005114 | heparan sulfate D-glucosaminyl |
| HS3ST3A1 | NM_006042 | heparan sulfate D-glucosaminyl |
| HS6ST2 | NM_147175 | heparan sulfate 6-O-sulfotransferase 2 |
| HSPA9B | NM_004134 | heat shock 70 kDa protein 9B precursor |
| HSPC049 | NM_014149 | HSPC049 protein |
| HTLF | NM_002158 | T-cell leukemia virus enhancer factor |
| HTR1D | NM_000864 | 5-hydroxytryptamine (serotonin) receptor 1D |
| HTR2B | NM_000867 | 5-hydroxytryptamine (serotonin) receptor 2B |
| HTR2C | NM_000868 | 5-hydroxytryptamine (serotonin) receptor 2C |
| HUNK | NM_014586 | hormonally upregulated Neu-associated kinase |
| HYOU1 | NM_006389 | oxygen regulated protein precursor |
| HYPK | NM_016400 | Huntingtin interacting protein K |
| ICK | NM_014920 | intestinal cell kinase |
| ID2 | NM_002166 | inhibitor of DNA binding 2 |
| IDH1 | NM_005896 | isocitrate dehydrogenase 1 (NADP+), soluble |
| IFIT5 | NM_012420 | interferon-induced protein with |
| IFNAR1 | NM_000629 | interferon-alpha receptor 1 precursor |
| IFT81 | NM_031473 | carnitine deficiency-associated, expressed in |
| IGF1 | NM_000618 | insulin-like growth factor 1 (somatomedin C) |
| IGF2BP1 | NM_006546 | insulin-like growth factor 2 mRNA binding |
| IGF2R | NM_000876 | insulin-like growth factor 2 receptor |
| IGSF1 | NM_205833 | immunoglobulin superfamily, member 1 |
| | | isoform 2 |
| IGSF11 | NM_001015887 | immunoglobulin superfamily, member 11 |
| | | isoform b |
| IHPK1 | NM_001006115 | inositol hexaphosphate kinase 1 isoform 2 |
| IKBKB | NM_001556 | inhibitor of kappa light polypeptide gene |
| IKIP | NM_201613 | IKK interacting protein isoform 3.1 |
| IL16 | NM_004513 | interleukin 16 isoform 1 precursor |
| IL6ST | NM_175767 | interleukin 6 signal transducer isoform 2 |
| IL8 | NM_000584 | interleukin 8 precursor |
| IMMP2L | NM_032549 | IMP2 inner mitochondrial membrane protease- |
| | | like |
| IMPA1 | NM_005536 | inositol(myo)-1(or 4)-monophosphatase 1 |
| IMPG1 | NM_001563 | interphotoreceptor matrix proteoglycan 1 |
| ING2 | NM_001564 | inhibitor of growth family, member 1-like |
| INSM2 | NM_032594 | insulinoma-associated protein IA-6 |
| INTS7 | NM_015434 | integrator complex subunit 7 |
| IPO8 | NM_006390 | importin 8 |
| IQSEC2 | NM_015075 | IQ motif and Sec7 domain 2 |
| IRF4 | NM_002460 | interferon regulatory factor 4 |
| IRS1 | NM_005544 | insulin receptor substrate 1 |
| IRX5 | NM_005853 | iroquois homeobox protein 5 |
| ISOC1 | NM_016048 | isochorismatase domain containing 1 |
| ITGA10 | NM_003637 | integrin, alpha 10 precursor |
| ITGA4 | NM_000885 | integrin alpha 4 precursor |
| ITGAV | NM_002210 | integrin alpha-V precursor |
| ITGB1 | NM_033666 | integrin beta 1 isoform 1B precursor |
| ITGB3 | NM_000212 | integrin beta chain, beta 3 precursor |
| ITIH5L | NM_198510 | hypothetical protein LOC347365 |
| ITM2B | NM_021999 | integral membrane protein 2B |
| ITPR1 | NM_002222 | inositol 1,4,5-triphosphate receptor, type 1 |
| ITSN1 | NM_001001132 | intersectin 1 isoform ITSN-s |
| ITSN2 | NM_006277 | intersectin 2 isoform 1 |
| IVL | NM_005547 | involucrin |
| IXL | NM_017592 | intersex-like |
| JAG2 | NM_002226 | jagged 2 isoform a precursor |
| JAM3 | NM_032801 | junctional adhesion molecule 3 precursor |
| JARID1A | NM_005056 | retinoblastoma binding protein 2 |
| JAZF1 | NM_175061 | juxtaposed with another zinc finger gene 1 |
| JMJD1B | NM_016604 | jumonji domain containing 1B |
| JMJD2A | NM_014663 | jumonji domain containing 2A |
| JUN | NM_002228 | v-jun avian sarcoma virus 17 oncogene |
| | | homolog |
| KATNAL1 | NM_001014380 | katanin p60 subunit A-like 1 |
| KBTBD3 | NM_152433 | BTB and kelch domain containing 3 |
| KBTBD6 | NM_152903 | kelch repeat and BTB (POZ) domain- |
| | | containing 6 |
| KCNA3 | NM_002232 | potassium voltage-gated channel, shaker- |
| | | related |
| KCND2 | NM_012281 | potassium voltage-gated channel, Shal-related |
| KCNE1 | NM_000219 | potassium voltage-gated channel, Isk-related |
| KCNE3 | NM_005472 | potassium voltage-gated channel, Isk-related |
| KCNJ13 | NM_002242 | potassium inwardly-rectifying channel J13 |
| KCNK2 | NM_001017424 | potassium channel, subfamily K, member 2 |
| | | isoform |
| KCNMA1 | NM_002247 | large conductance calcium-activated potassium |
| KCNQ4 | NM_004700 | potassium voltage-gated channel KQT-like |
| | | protein |
| KCTD12 | NM_138444 | potassium channel tetramerisation domain |
| KCTD2 | NM_015353 | potassium channel tetramerisation domain |
| KCTD8 | NM_198353 | potassium channel tetramerisation domain |
| KDELC1 | NM_024089 | KDEL (Lys-Asp-Glu-Leu) containing 1 |
| KDR | NM_002253 | kinase insert domain receptor (a type III |
| KENAE | NM_176816 | hypothetical protein LOC202243 |
| KIAA0040 | NM_014656 | hypothetical protein LOC9674 |
| KIAA0101 | NM_001029989 | hypothetical protein LOC9768 isoform 2 |
| KIAA0152 | NM_014730 | hypothetical protein LOC9761 |
| KIAA0182 | NM_014615 | hypothetical protein LOC23199 |
| KIAA0247 | NM_014734 | hypothetical protein LOC9766 |
| KIAA0256 | NM_014701 | hypothetical protein LOC9728 |
| KIAA0286 | NM_015257 | hypothetical protein LOC23306 |
| KIAA0319 | NM_014809 | KIAA0319 |
| KIAA0355 | NM_014686 | hypothetical protein LOC9710 |
| KIAA0423 | NM_015091 | hypothetical protein LOC23116 |
| KIAA0446 | NM_014655 | hypothetical protein LOC9673 |
| KIAA0553 | NM_001002909 | hypothetical protein LOC23131 |
| KIAA0644 | NM_014817 | hypothetical protein LOC9865 |
| KIAA0853 | NM_015070 | KIAA0853 |
| KIAA0895 | NM_015314 | hypothetical protein LOC23366 |
| KIAA1012 | NM_014939 | hypothetical protein LOC22878 |
| KIAA1024 | NM_015206 | hypothetical protein LOC23251 |
| KIAA1033 | NM_015275 | hypothetical protein LOC23325 |
| KIAA1128 | NM_018999 | granule cell antiserum positive 14 |
| KIAA1244 | NM_020340 | hypothetical protein LOC57221 |
| KIAA1274 | NM_014431 | KIAA1274 |
| KIAA1333 | NM_017769 | hypothetical protein LOC55632 |
| KIAA1432 | NM_020829 | hypothetical protein LOC57589 |
| KIAA1559 | NM_020917 | zinc finger protein 14-like |
| KIAA1576 | NM_020927 | hypothetical protein LOC57687 |
| KIAA1600 | NM_020940 | hypothetical protein LOC57700 |
| KIAA1715 | NM_030650 | Lunapark |
| KIAA1841 | NM_032506 | KIAA1841 protein |
| KIAA1853 | NM_194286 | KIAA1853 protein |
| KIAA1909 | NM_052909 | hypothetical protein LOC153478 |
| KIAA2018 | NM_001009899 | hypothetical protein LOC205717 |
| KITLG | NM_000899 | KIT ligand isoform b precursor |
| KL | NM_004795 | klotho isoform a |
| KLF11 | NM_003597 | Kruppel-like factor 11 |
| KLF12 | NM_007249 | Kruppel-like factor 12 isoform a |
| KLF13 | NM_015995 | Kruppel-like factor 13 |
| KLF4 | NM_004235 | Kruppel-like factor 4 (gut) |
| KLF9 | NM_001206 | Kruppel-like factor 9 |
| KLHDC1 | NM_172193 | kelch domain containing 1 |
| KLHDC5 | NM_020782 | kelch domain containing 5 |
| KLHL12 | NM_021633 | kelch-like 12 |
| KLHL14 | NM_020805 | kelch-like 14 |
| KLHL3 | NM_017415 | kelch-like 3 (Drosophila) |
| KLHL9 | NM_018847 | kelch-like 9 |
| KRAS | NM_004985 | c-K-ras2 protein isoform b |
| KRT12 | NM_000223 | keratin 12 |
| KRTAP3-2 | NM_031959 | keratin associated protein 3.2 |
| KSR1 | NM_014238 | kinase suppressor of ras |
| KYNU | NM_003937 | kynureninase (L-kynurenine hydrolase) |
| | | isoform a |
| LAMC1 | NM_002293 | laminin, gamma 1 precursor |
| LARP2 | NM_018078 | La ribonucleoprotein domain family member 2 |
| LASS6 | NM_203463 | longevity assurance homolog 6 |
| LCP1 | NM_002298 | L-plastin |
| LEMD3 | NM_014319 | LEM domain containing 3 |
| LEPR | NM_001003679 | leptin receptor isoform 2 |
| LEPROTL1 | NM_015344 | leptin receptor overlapping transcript-like 1 |
| LHFP | NM_005780 | lipoma HMGIC fusion partner |
| LHFPL2 | NM_005779 | lipoma HMGIC fusion partner-like 2 |
| LHX9 | NM_001014434 | LIM homeobox 9 isoform 2 |
| LIMK1 | NM_002314 | LIM domain kinase 1 |
| LIN28B | NM_001004317 | lin-28 homolog B |
| LIN7B | NM_022165 | lin-7 homolog B |
| LKAP | NM_014647 | limkain b1 |
| LLGL1 | NM_004140 | lethal giant larvae homolog 1 |
| LMO7 | NM_005358 | LIM domain only 7 |
| LNX2 | NM_153371 | PDZ domain containing ring finger 1 |
| LOC124491 | NM_145254 | hypothetical protein LOC124491 |
| LOC128977 | NM_173793 | hypothetical protein LOC128977 |
| LOC133957 | NM_145265 | hypothetical protein LOC133957 |
| LOC138046 | NM_173848 | hypothetical protein LOC138046 |
| LOC144501 | NM_182507 | hypothetical protein LOC144501 |
| LOC153364 | NM_203406 | similar to metallo-beta-lactamase superfamily |
| LOC155060 | NM_001004302 | hypothetical protein LOC155060 |
| LOC158160 | NM_182829 | 17-beta-hydroxysteroid dehydrogenase type |
| LOC196394 | NM_207337 | hypothetical protein LOC196394 |
| LOC203547 | NM_001017980 | hypothetical protein LOC203547 |
| LOC283514 | NM_198849 | hypothetical protein LOC283514 |
| LOC284757 | NM_001004305 | hypothetical protein LOC284757 |
| LOC285429 | NM_001029955 | hypothetical protein LOC285429 |
| LOC339524 | NM_207357 | hypothetical protein LOC339524 |
| LOC339745 | NM_001001664 | hypothetical protein LOC339745 |
| LOC340843 | NM_001013629 | hypothetical protein LOC340843 |
| LOC387646 | NM_001006604 | hypothetical protein LOC387646 |
| LOC387758 | NM_203371 | hypothetical protein LOC387758 |
| LOC388272 | NM_001001436 | hypothetical protein LOC388272 |
| LOC388335 | NM_001004313 | hypothetical protein LOC388335 |
| LOC389432 | NM_001030060 | hypothetical protein LOC389432 |
| LOC389834 | NM_001013655 | hypothetical protein LOC389834 |
| LOC389936 | NM_001013656 | hypothetical protein LOC389936 |
| LOC390980 | NM_001023563 | similar to Zinc finger protein 264 |
| LOC399898 | NM_001013666 | hypothetical protein LOC399898 |
| LOC399947 | NM_207645 | hypothetical protein LOC399947 |
| LOC401252 | NM_001013681 | hypothetical protein LOC401252 |
| LOC401431 | NM_001008745 | hypothetical protein LOC401431 |
| LOC401720 | NM_001013690 | hypothetical protein LOC401720 |
| LOC440905 | NM_001013711 | hypothetical protein LOC440905 |
| LOC440944 | NM_001013713 | hypothetical protein LOC440944 |
| LOC441108 | NM_001013717 | hypothetical protein LOC441108 |
| LOC441136 | NM_001013719 | hypothetical protein LOC441136 |
| LOC441233 | NM_001013724 | hypothetical protein LOC441233 |
| LOC441426 | NM_001013727 | hypothetical protein LOC441426 |
| LOC51333 | NM_016643 | mesenchymal stem cell protein DSC43 |
| LOC619208 | NM_001033564 | hypothetical protein LOC619208 |
| LOC90355 | NM_033211 | hypothetical protein LOC90355 |
| LOC93622 | NM_138699 | hypothetical protein LOC93622 |
| LOX | NM_002317 | lysyl oxidase preproprotein |
| LPGAT1 | NM_014873 | lysophosphatidylglycerol acyltransferase 1 |
| LPHN2 | NM_012302 | latrophilin 2 precursor |
| LPIN1 | NM_145693 | lipin 1 |
| LPIN2 | NM_014646 | lipin 2 |
| LPPR4 | NM_014839 | plasticity related gene 1 |
| LRAT | NM_004744 | lecithin retinol acyltransferase |
| LRCH1 | NM_015116 | leucine-rich repeats and calponin homology |
| | | (CH) |
| LRIG1 | NM_015541 | leucine-rich repeats and immunoglobulin-like |
| LRP1 | NM_002332 | low density lipoprotein-related protein 1 |
| LRP2BP | NM_018409 | LRP2 binding protein |
| LRP4 | NM_002334 | low density lipoprotein receptor-related |
| | | protein |
| LRRC15 | NM_130830 | leucine rich repeat containing 15 |
| LRRC19 | NM_022901 | leucine rich repeat containing 19 |
| LRRC40 | NM_017768 | leucine rich repeat containing 40 |
| LRRC8A | NM_019594 | leucine-rich repeat-containing 8 |
| LRRFIP1 | NM_004735 | leucine rich repeat (in FLII) interacting |
| LRRTM3 | NM_178011 | leucine rich repeat transmembrane neuronal 3 |
| LRRTM4 | NM_024993 | leucine rich repeat transmembrane neuronal 4 |
| LY6K | NM_017527 | lymphocyte antigen 6 complex, locus K |
| LY75 | NM_002349 | lymphocyte antigen 75 |
| LYCAT | NM_001002257 | lysocardiolipin acyltransferase isoform 2 |
| LYPLA1 | NM_006330 | lysophospholipase I |
| LYPLA2 | NM_007260 | lysophospholipase II |
| LYSMD4 | NM_152449 | hypothetical protein LOC145748 |
| M6PR | NM_002355 | cation-dependent mannose-6-phosphate |
| | | receptor |
| M6PRBP1 | NM_005817 | mannose 6 phosphate receptor binding protein 1 |
| MAB21L1 | NM_005584 | mab-21-like protein 1 |
| MAFG | NM_002359 | v-maf musculoaponeurotic fibrosarcoma |
| | | oncogene |
| MAGEA12 | NM_005367 | melanoma antigen family A, 12 |
| MAGEB18 | NM_173699 | melanoma antigen family B, 18 |
| MAGEC2 | NM_016249 | melanoma antigen family C, 2 |
| MAGOH | NM_002370 | mago-nashi homolog |
| MAK | NM_005906 | male germ cell-associated kinase |
| MALT1 | NM_006785 | mucosa associated lymphoid tissue lymphoma |
| MAMDC2 | NM_153267 | MAM domain containing 2 |
| MAML1 | NM_014757 | mastermind-like 1 |
| MAP2 | NM_002374 | microtubule-associated protein 2 isoform 1 |
| MAP2K5 | NM_002757 | mitogen-activated protein kinase kinase 5 |
| MAP2K6 | NM_002758 | mitogen-activated protein kinase kinase 6 |
| MAP4K3 | NM_003618 | mitogen-activated protein kinase kinase kinase |
| MAP4K4 | NM_004834 | mitogen-activated protein kinase kinase kinase |
| MAPK13 | NM_002754 | mitogen-activated protein kinase 13 |
| MAPK7 | NM_002749 | mitogen-activated protein kinase 7 isoform 1 |
| MAPK9 | NM_002752 | mitogen-activated protein kinase 9 isoform 1 |
| MAPRE1 | NM_012325 | microtubule-associated protein, RP/EB family, |
| MARCKS | NM_002356 | myristoylated alanine-rich protein kinase C |
| MARCKSL1 | NM_023009 | MARCKS-like 1 |
| MARVELD1 | NM_031484 | MARVEL domain containing 1 |
| MASA | NM_021204 | E-1 enzyme |
| MATN3 | NM_002381 | matrilin 3 precursor |
| MATR3 | NM_018834 | matrin 3 |
| MBL2 | NM_000242 | soluble mannose-binding lectin precursor |
| MBNL1 | NM_021038 | muscleblind-like 1 isoform a |
| MBP | NM_001025100 | Golli-mbp isoform 2 |
| MBTD1 | NM_017643 | mbt domain containing 1 |
| MCFD2 | NM_139279 | multiple coagulation factor deficiency 2 |
| MCM10 | NM_018518 | minichromosome maintenance protein 10 |
| | | isoform 2 |
| MCM8 | NM_032485 | minichromosome maintenance protein 8 |
| | | isoform 1 |
| MEF2D | NM_005920 | MADS box transcription enhancer factor 2, |
| MEGF10 | NM_032446 | MEGF10 protein |
| MEGF11 | NM_032445 | MEGF11 protein |
| METTL7A | NM_014033 | hypothetical protein LOC25840 |
| MFAP5 | NM_003480 | microfibrillar associated protein 5 |
| MFSD4 | NM_181644 | hypothetical protein DKFZp761N1114 |
| MGAT2 | NM_001015883 | mannosyl (alpha-1,6-)-glycoprotein |
| MGC13017 | NM_080656 | hypothetical protein LOC91368 |
| MGC26694 | NM_178526 | hypothetical protein LOC284439 |
| MGC26816 | NM_152613 | hypothetical protein LOC164684 |
| MGC3207 | NM_032285 | hypothetical protein LOC84245 isoform 2 |
| MGC33926 | NM_152390 | hypothetical protein LOC130733 |
| MGC34646 | NM_173519 | hypothetical protein LOC157807 |
| MGC35048 | NM_153208 | hypothetical protein LOC124152 |
| MGC3731 | NM_024313 | hypothetical protein LOC79159 |
| MGC42090 | NM_152774 | hypothetical protein LOC256130 |
| MGC4268 | NM_031445 | hypothetical protein LOC83607 |
| MGC5297 | NM_024091 | hypothetical protein LOC79072 |
| MGC87631 | NM_001004306 | hypothetical protein LOC339184 |
| MGC9850 | NM_152705 | hypothetical protein MGC9850 |
| MIB1 | NM_020774 | mindbomb homolog 1 |
| MIER1 | NM_020948 | mesoderm induction early response 1 |
| MIP | NM_012064 | major intrinsic protein of lens fiber |
| MITF | NM_000248 | microphthalmia-associated transcription factor |
| MKI67 | NM_002417 | antigen identified by monoclonal antibody Ki- |
| | | 67 |
| MKL2 | NM_014048 | megakaryoblastic leukemia 2 protein |
| MKLN1 | NM_013255 | muskelin 1, intracellular mediator containing |
| MKNK2 | NM_199054 | MAP kinase-interacting serine/threonine |
| | | kinase 2 |
| MKRN1 | NM_013446 | makorin, ring finger protein, 1 |
| MLLT10 | NM_001009569 | myeloid/lymphoid or mixed-lineage leukemia |
| MLLT11 | NM_006818 | MLLT11 protein |
| MLR1 | NM_153686 | transcription factor MLR1 |
| MMD | NM_012329 | monocyte to macrophage |
| MMD2 | NM_198403 | monocyte-to-macrophage differentiation factor 2 |
| MMP19 | NM_001032360 | matrix metalloproteinase 19 isoform 2 |
| | | precursor |
| MOBK1B | NM_018221 | Mob4B protein |
| MOBKL1A | NM_173468 | MOB1, Mps One Binder kinase activator-like |
| | | 1A |
| MOBKL2B | NM_024761 | MOB1, Mps One Binder kinase activator-like |
| | | 2B |
| MOCS2 | NM_004531 | molybdopterin synthase large subunit |
| | | MOCS2B |
| MORC3 | NM_015358 | MORC family CW-type zinc finger 3 |
| MOSC2 | NM_017898 | MOCO sulphurase C-terminal domain |
| | | containing 2 |
| MOSPD2 | NM_152581 | motile sperm domain containing 2 |
| MPP4 | NM_033066 | membrane protein, palmitoylated 4 |
| MPP5 | NM_022474 | membrane protein, palmitoylated 5 |
| MPPED1 | NM_001585 | hypothetical protein LOC758 |
| MRAS | NM_012219 | muscle RAS oncogene homolog |
| M-RIP | NM_015134 | myosin phosphatase-Rho interacting protein |
| MRO | NM_031939 | maestro |
| MRPL27 | NM_148571 | mitochondrial ribosomal protein L27 isoform b |
| MRPS25 | NM_022497 | mitochondrial ribosomal protein S25 |
| MS4A2 | NM_000139 | membrane-spanning 4-domains, subfamily A, |
| | | member |
| MSL2L1 | NM_018133 | ring finger protein 184 |
| MSN | NM_002444 | moesin |
| MSR1 | NM_002445 | macrophage scavenger receptor 1 isoform type 2 |
| MTAP | NM_002451 | 5′-methylthioadenosine phosphorylase |
| MTCP1 | NM_001018025 | mature T-cell proliferation 1 isoform p13 |
| MTDH | NM_178812 | LYRIC/3D3 |
| MTERFD1 | NM_015942 | MTERF domain containing 1 |
| MTFR1 | NM_014637 | chondrocyte protein with a poly-proline region |
| MTHFR | NM_005957 | 5,10-methylenetetrahydrofolate reductase |
| MTMR1 | NM_003828 | myotubularin-related protein 1 |
| MTMR12 | NM_019061 | myotubularin related protein 12 |
| MTMR9 | NM_015458 | myotubularin-related protein 9 |
| MTUS1 | NM_001001924 | mitochondrial tumor suppressor 1 isoform 1 |
| MUTED | NM_201280 | muted |
| MXD1 | NM_002357 | MAX dimerization protein 1 |
| MXD4 | NM_006454 | MAD4 |
| MYB | NM_005375 | v-myb myeloblastosis viral oncogene homolog |
| MYC | NM_002467 | myc proto-oncogene protein |
| MYCN | NM_005378 | v-myc myelocytomatosis viral related |
| | | oncogene, |
| MYEOV | NM_138768 | myeloma overexpressed |
| MYLK | NM_005965 | myosin light chain kinase isoform 6 |
| NAB1 | NM_005966 | NGFI-A binding protein 1 |
| NANOS1 | NM_001009553 | nanos homolog 1 isoform 2 |
| NANOS2 | NM_001029861 | nanos homolog 2 |
| NAP1L2 | NM_021963 | nucleosome assembly protein 1-like 2 |
| NAP1L5 | NM_153757 | nucleosome assembly protein 1-like 5 |
| NARF | NM_012336 | nuclear prelamin A recognition factor isoform a |
| NARG1L | NM_018527 | NMDA receptor regulated 1-like protein |
| | | isoform |
| NAV1 | NM_020443 | neuron navigator 1 |
| NBR1 | NM_005899 | neighbor of BRCA1 gene 1 |
| NCAM1 | NM_181351 | neural cell adhesion molecule 1 isoform 2 |
| NCKAP1 | NM_013436 | NCK-associated protein 1 isoform 1 |
| NCOA1 | NM_003743 | nuclear receptor coactivator 1 isoform 1 |
| NCOA2 | NM_006540 | nuclear receptor coactivator 2 |
| NCOA3 | NM_006534 | nuclear receptor coactivator 3 isoform b |
| NCOA4 | NM_005437 | nuclear receptor coactivator 4 |
| NCOR2 | NM_006312 | nuclear receptor co-repressor 2 |
| NDN | NM_002487 | necdin |
| NDST1 | NM_001543 | N-deacetylase/N-sulfotransferase (heparan |
| NDUFS1 | NM_005006 | NADH dehydrogenase (ubiquinone) Fe—S |
| | | protein 1, |
| NDUFS4 | NM_002495 | NADH dehydrogenase (ubiquinone) Fe—S |
| | | protein 4, |
| NEDD4 | NM_006154 | neural precursor cell expressed, |
| | | developmentally |
| NEDD4L | NM_015277 | ubiquitin-protein ligase NEDD4-like |
| NEDD8 | NM_006156 | neural precursor cell expressed, |
| | | developmentally |
| NEGR1 | NM_173808 | neuronal growth regulator 1 |
| NFASC | NM_015090 | neurofascin precursor |
| NFATC2IP | NM_032815 | nuclear factor of activated T-cells, |
| NFIA | NM_005595 | nuclear factor I/A |
| NFYA | NM_002505 | nuclear transcription factor Y, alpha isoform 1 |
| NGEF | NM_019850 | neuronal guanine nucleotide exchange factor |
| NGRN | NM_001033088 | mesenchymal stem cell protein DSC92 |
| | | isoform 2 |
| NHLH1 | NM_005598 | nescient helix loop helix 1 |
| NIN | NM_020921 | ninein isoform 2 |
| NIPA1 | NM_144599 | non-imprinted in Prader-Willi/Angelman |
| | | syndrome |
| NIPBL | NM_133433 | delangin isoform A |
| NIPSNAP3B | NM_018376 | nipsnap homolog 3B |
| NKD1 | NM_033119 | naked cuticle homolog 1 |
| NLE1 | NM_001014445 | Notchless gene homolog isoform b |
| NLGN4X | NM_020742 | X-linked neuroligin 4 |
| NLGN4Y | NM_014893 | neuroligin 4, Y-linked |
| NMNAT2 | NM_015039 | nicotinamide mononucleotide |
| | | adenylyltransferase |
| NOG | NM_005450 | noggin precursor |
| NOPE | NM_020962 | DDM36 |
| NOTCH1 | NM_017617 | notch1 preproprotein |
| NOVA1 | NM_002515 | neuro-oncological ventral antigen 1 isoform 1 |
| N-PAC | NM_032569 | cytokine-like nuclear factor n-pac |
| NPAT | NM_002519 | nuclear protein, ataxia-telangiectasia locus |
| NPC1 | NM_000271 | Niemann-Pick disease, type C1 |
| NPNT | NM_001033047 | nephronectin |
| NPY2R | NM_000910 | neuropeptide Y receptor Y2 |
| NR3C1 | NM_000176 | nuclear receptor subfamily 3, group C, |
| | | member 1 |
| NR4A2 | NM_006186 | nuclear receptor subfamily 4, group A, |
| | | member 2 |
| NR5A2 | NM_003822 | nuclear receptor subfamily 5, group A, |
| | | member 2 |
| NRBF2 | NM_030759 | nuclear receptor binding factor 2 |
| NRBP1 | NM_013392 | nuclear receptor binding protein |
| NRIP1 | NM_003489 | receptor interacting protein 140 |
| NRP1 | NM_003873 | neuropilin 1 isoform a |
| NRP2 | NM_003872 | neuropilin 2 isoform 2 precursor |
| NSMAF | NM_003580 | neutral sphingomyelinase (N-SMase) |
| | | activation |
| NSUN2 | NM_017755 | NOL1/NOP2/Sun domain family 2 protein |
| NT5DC1 | NM_152729 | 5′-nucleotidase, cytosolic II-like 1 protein |
| NTF3 | NM_002527 | neurotrophin 3 precursor |
| NTRK2 | NM_001007097 | neurotrophic tyrosine kinase, receptor, type 2 |
| NUBPL | NM_025152 | nucleotide binding protein-like |
| NUDCD1 | NM_032869 | NudC domain containing 1 |
| NUDCD3 | NM_015332 | NudC domain containing 3 |
| NUDT21 | NM_007006 | cleavage and polyadenylation specific factor 5 |
| NUFIP2 | NM_020772 | 82-kD FMRP Interacting Protein |
| NUMB | NM_001005743 | numb homolog isoform 1 |
| NUP153 | NM_005124 | nucleoporin 153 kDa |
| NUP35 | NM_001008544 | nucleoporin 35 kDa isoform b |
| NUP43 | NM_198887 | nucleoporin 43 kDa |
| NUPL1 | NM_001008564 | nucleoporin like 1 isoform b |
| NY-REN-7 | NM_173663 | hypothetical protein LOC285596 |
| OBFC2B | NM_024068 | hypothetical protein LOC79035 |
| OCLN | NM_002538 | occludin |
| OGN | NM_014057 | osteoglycin preproprotein |
| OGT | NM_003605 | O-linked GlcNAc transferase isoform 3 |
| OLIG3 | NM_175747 | oligodendrocyte transcription factor 3 |
| OPA1 | NM_015560 | optic atrophy 1 isoform 1 |
| OPHN1 | NM_002547 | oligophrenin 1 |
| OPRM1 | NM_001008503 | opioid receptor, mu 1 isoform MOR-1O |
| ORC5L | NM_002553 | origin recognition complex subunit 5 isoform 1 |
| OSBP | NM_002556 | oxysterol binding protein |
| OSBPL11 | NM_022776 | oxysterol-binding protein-like protein 11 |
| OSBPL8 | NM_001003712 | oxysterol-binding protein-like protein 8 |
| | | isoform |
| OSGEPL1 | NM_022353 | O-sialoglycoprotein endopeptidase-like 1 |
| OSMR | NM_003999 | oncostatin M receptor |
| OSR1 | NM_145260 | odd-skipped related 1 |
| OSRF | NM_012382 | osmosis responsive factor |
| OSTM1 | NM_014028 | osteopetrosis associated transmembrane |
| | | protein |
| OTUD4 | NM_199324 | OTU domain containing 4 protein isoform 1 |
| OTUD6B | NM_016023 | OTU domain containing 6B |
| OXCT1 | NM_000436 | 3-oxoacid CoA transferase 1 precursor |
| OXGR1 | NM_080818 | oxoglutarate (alpha-ketoglutarate) receptor 1 |
| OXR1 | NM_181354 | oxidation resistance 1 |
| P15RS | NM_018170 | hypothetical protein FLJ10656 |
| P18SRP | NM_173829 | P18SRP protein |
| P2RY1 | NM_002563 | purinergic receptor P2Y1 |
| PAG1 | NM_018440 | phosphoprotein associated with |
| | | glycosphingolipid |
| PAIP1 | NM_006451 | poly(A) binding protein interacting protein 1 |
| PAIP2 | NM_001033112 | poly(A) binding protein interacting protein 2 |
| PAK2 | NM_002577 | p21-activated kinase 2 |
| PAK6 | NM_020168 | p21-activated kinase 6 |
| PAK7 | NM_020341 | p21-activated kinase 7 |
| PALM2-AKAP2 | NM_007203 | PALM2-AKAP2 protein isoform 1 |
| PAM | NM_000919 | peptidylglycine alpha-amidating |
| | | monooxygenase |
| PAN3 | NM_175854 | PABP1-dependent poly A-specific |
| | | ribonuclease |
| PAP2D | NM_001010861 | phosphatidic acid phosphatase type 2d isoform 2 |
| PAPD5 | NM_022447 | PAP associated domain containing 5 |
| PAPOLB | NM_020144 | poly(A) polymerase beta (testis specific) |
| PAPOLG | NM_022894 | poly(A) polymerase gamma |
| PAQR5 | NM_017705 | membrane progestin receptor gamma |
| PARP14 | NM_017554 | poly (ADP-ribose) polymerase family, |
| | | member 14 |
| PARP6 | NM_020213 | poly (ADP-ribose) polymerase family, |
| | | member 6 |
| PCAF | NM_003884 | p300/CBP-associated factor |
| PCDH10 | NM_032961 | protocadherin 10 isoform 1 precursor |
| PCDH21 | NM_033100 | protocadherin 21 precursor |
| PCDH7 | NM_032456 | protocadherin 7 isoform b precursor |
| PCDH8 | NM_002590 | protocadherin 8 isoform 1 precursor |
| PCDHAC1 | NM_031882 | protocadherin alpha subfamily C, 1 isoform 2 |
| PCDHB12 | NM_018932 | protocadherin beta 12 precursor |
| PCDHB14 | NM_018934 | protocadherin beta 14 precursor |
| PCDHB16 | NM_020957 | protocadherin beta 16 precursor |
| PCMTD1 | NM_052937 | hypothetical protein LOC115294 |
| PCNP | NM_020357 | PEST-containing nuclear protein |
| PCSK2 | NM_002594 | proprotein convertase subtilisin/kexin type 2 |
| PCSK6 | NM_138323 | paired basic amino acid cleaving system 4 |
| PCTK1 | NM_006201 | PCTAIRE protein kinase 1 |
| PCTK2 | NM_002595 | PCTAIRE protein kinase 2 |
| PCYOX1 | NM_016297 | prenylcysteine oxidase 1 |
| PDC | NM_002597 | phosducin isoform a |
| PDCD10 | NM_007217 | programmed cell death 10 |
| PDCD4 | NM_014456 | programmed cell death 4 isoform 1 |
| PDCD6IP | NM_013374 | programmed cell death 6 interacting protein |
| PDE10A | NM_006661 | phosphodiesterase 10A |
| PDE5A | NM_001083 | phosphodiesterase 5A isoform 1 |
| PDE8B | NM_001029851 | phosphodiesterase 8B isoform 3 |
| PDIK1L | NM_152835 | PDLIM1 interacting kinase 1 like |
| PELI2 | NM_021255 | pellino 2 |
| PFN2 | NM_053024 | profilin 2 isoform a |
| PFTK1 | NM_012395 | PFTAIRE protein kinase 1 |
| PGAP1 | NM_024989 | GPI deacylase |
| PGM2L1 | NM_173582 | phosphoglucomutase 2-like 1 |
| PHACTR2 | NM_014721 | phosphatase and actin regulator 2 |
| PHCA | NM_018367 | phytoceramidase, alkaline |
| PHF16 | NM_014735 | PHD finger protein 16 |
| PHF20L1 | NM_016018 | PHD finger protein 20-like 1 isoform 1 |
| PHF21A | NM_016621 | BRAF35/HDAC2 complex |
| PHF21B | NM_138415 | PHD finger protein 21B |
| PHF6 | NM_001015877 | PHD finger protein 6 isoform 1 |
| PHLDB1 | NM_015157 | pleckstrin homology-like domain, family B, |
| PHOSPHO1 | NM_178500 | phosphatase, orphan 1 |
| PHTF2 | NM_020432 | putative homeodomain transcription factor 2 |
| PI15 | NM_015886 | protease inhibitor 15 preproprotein |
| PIGM | NM_145167 | PIG-M mannosyltransferase |
| PIK3C2G | NM_004570 | phosphoinositide-3-kinase, class 2, gamma |
| PIK3R3 | NM_003629 | phosphoinositide-3-kinase, regulatory subunit 3 |
| PIK4CB | NM_002651 | phosphatidylinositol 4-kinase, catalytic, beta |
| PIM2 | NM_006875 | pim-2 oncogene |
| PIN1 | NM_006221 | protein (peptidyl-prolyl cis/trans isomerase) |
| PIP3-E | NM_015553 | phosphoinositide-binding protein PIP3-E |
| PIP5K2C | NM_024779 | phosphatidylinositol-4-phosphate 5-kinase, |
| | | type |
| PIP5K3 | NM_001002881 | phosphatidylinositol-3- |
| PISD | NM_014338 | phosphatidylserine decarboxylase |
| PITPNA | NM_006224 | phosphatidylinositol transfer protein, alpha |
| PKD1 | NM_000296 | polycystin 1 isoform 2 precursor |
| PKD2 | NM_000297 | polycystin 2 |
| PKHD1 | NM_138694 | polyductin isoform 1 |
| PKIA | NM_006823 | cAMP-dependent protein kinase inhibitor |
| | | alpha |
| PKMYT1 | NM_004203 | protein kinase Myt1 isoform 1 |
| PKP1 | NM_000299 | plakophilin 1 isoform 1b |
| PLAA | NM_004253 | phosphohipase A2-activating protein isoform 2 |
| PLAG1 | NM_002655 | pleiomorphic adenoma gene 1 |
| PLCG1 | NM_002660 | phospholipase C gamma 1 isoform a |
| PLCXD3 | NM_001005473 | phosphatidylinositol-specific phospholipase C, X |
| PLDN | NM_012388 | pallidin |
| PLEKHA6 | NM_014935 | phosphoinositol 3-phosphate-binding protein-3 |
| PLEKHK1 | NM_145307 | pleckstrin homology domain containing, |
| | | family K |
| PLGLB1 | NM_001032392 | plasminogen-like B1 |
| PLGLB2 | NM_002665 | plasminogen-related protein B2 |
| PLK2 | NM_006622 | polo-like kinase 2 |
| PLS1 | NM_002670 | plastin 1 |
| PLS3 | NM_005032 | plastin 3 |
| PMAIP1 | NM_021127 | phorbol-12-myristate-13-acetate-induced |
| | | protein |
| PMM1 | NM_002676 | phosphomannomutase 1 |
| PMP22 | NM_000304 | peripheral myelin protein 22 |
| PNMA2 | NM_007257 | paraneoplastic antigen MA2 |
| PNRC2 | NM_017761 | proline-rich nuclear receptor coactivator 2 |
| POLK | NM_016218 | polymerase (DNA directed) kappa |
| POLR1B | NM_019014 | RNA polymerase I polypeptide B |
| PPARA | NM_001001928 | peroxisome proliferative activated receptor, |
| PPARGC1A | NM_013261 | peroxisome proliferative activated receptor |
| PPFIA1 | NM_003626 | PTPRF interacting protein alpha 1 isoform b |
| PPFIBP1 | NM_003622 | PTPRF interacting protein binding protein 1 |
| PPIL4 | NM_139126 | peptidylprolyl isomerase-like 4 |
| PPM1B | NM_177968 | protein phosphatase 1B isoform 2 |
| PPM1E | NM_014906 | protein phosphatase 1E |
| PPM1F | NM_014634 | protein phosphatase 1F |
| PPM2C | NM_018444 | pyruvate dehydrogenase phosphatase |
| | | precursor |
| PPP1CB | NM_002709 | protein phosphatase 1, catalytic subunit, beta |
| PPP1R10 | NM_002714 | protein phosphatase 1, regulatory subunit 10 |
| PPP1R12B | NM_002481 | protein phosphatase 1, regulatory (inhibitor) |
| PPP1R16B | NM_015568 | protein phosphatase 1 regulatory inhibitor |
| PPP1R2 | NM_006241 | protein phosphatase 1, regulatory (inhibitor) |
| PPP1R3A | NM_002711 | protein phosphatase 1 glycogen-binding |
| PPP1R3D | NM_006242 | protein phosphatase 1, regulatory subunit 3D |
| PPP2CA | NM_002715 | protein phosphatase 2, catalytic subunit, alpha |
| PPP2R1B | NM_002716 | beta isoform of regulatory subunit A, protein |
| PPP2R2C | NM_020416 | gamma isoform of regulatory subunit B55, |
| | | protein |
| PPP2R3A | NM_002718 | protein phosphatase 2, regulatory subunit B″, |
| PPP2R5A | NM_006243 | protein phosphatase 2, regulatory subunit B |
| PPP2R5C | NM_002719 | gamma isoform of regulatory subunit B56, |
| | | protein |
| PPP2R5E | NM_006246 | epsilon isoform of regulatory subunit B56, |
| PPP3R2 | NM_147180 | protein phosphatase 3 regulatory subunit B, |
| | | beta |
| PPP4R2 | NM_174907 | protein phosphatase 4, regulatory subunit 2 |
| PQLC2 | NM_017765 | PQ loop repeat containing 2 isoform 1 |
| PRDM1 | NM_001198 | PR domain containing 1, with ZNF domain |
| | | isoform |
| PRDX2 | NM_005809 | peroxiredoxin 2 isoform a |
| PREX1 | NM_020820 | PREX1 protein |
| PRG-3 | NM_017753 | plasticity related gene 3 |
| PRICKLE2 | NM_198859 | prickle-like 2 |
| PRKAB1 | NM_006253 | AMP-activated protein kinase beta 1 |
| PRKAB2 | NM_005399 | AMP-activated protein kinase beta 2 |
| PRKAR1A | NM_002734 | cAMP-dependent protein kinase, regulatory |
| PRKAR2B | NM_002736 | cAMP-dependent protein kinase, regulatory |
| PRKCH | NM_006255 | protein kinase C, eta |
| PRKCQ | NM_006257 | protein kinase C, theta |
| PRKDC | NM_006904 | protein kinase, DNA-activated, catalytic |
| PRKG2 | NM_006259 | protein kinase, cGMP-dependent, type II |
| PRKY | NM_002760 | protein kinase, Y-linked |
| PRMT6 | NM_018137 | HMT1 hnRNP methyltransferase-like 6 |
| PRO0149 | NM_014117 | hypothetical protein LOC29035 |
| PROK2 | NM_021935 | prokineticin 2 |
| PROL1 | NM_021225 | basic proline-rich protein |
| PRPF38A | NM_032864 | PRP38 pre-mRNA processing factor 38 (yeast) |
| PRR3 | NM_025263 | proline-rich protein 3 |
| PSAT1 | NM_021154 | phosphoserine aminotransferase isoform 2 |
| PSCD1 | NM_004762 | pleckstrin homology, Sec7 and coiled/coil |
| PSCD3 | NM_004227 | pleckstrin homology, Sec7 and coiled/coil |
| PSCD4 | NM_013385 | pleckstrin homology, Sec7 and coiled/coil |
| PSD3 | NM_015310 | ADP-ribosylation factor guanine nucleotide |
| PSIP1 | NM_033222 | PC4 and SFRS1 interacting protein 1 isoform 2 |
| PSMD12 | NM_002816 | proteasome 26S non-ATPase subunit 12 |
| | | isoform 1 |
| PSRC2 | NM_144982 | hypothetical protein LOC196441 |
| PTBP1 | NM_002819 | polypyrimidine tract-binding protein 1 isoform |
| PTDSS1 | NM_014754 | phosphatidylserine synthase 1 |
| PTGER2 | NM_000956 | prostaglandin E receptor 2 (subtype EP2), |
| | | 53 kDa |
| PTP4A1 | NM_003463 | protein tyrosine phosphatase type IVA, |
| | | member 1 |
| PTPN11 | NM_002834 | protein tyrosine phosphatase, non-receptor |
| | | type |
| PTPN12 | NM_002835 | protein tyrosine phosphatase, non-receptor |
| | | type |
| PTPN13 | NM_006264 | protein tyrosine phosphatase, non-receptor |
| | | type |
| PTPN22 | NM_012411 | protein tyrosine phosphatase, non-receptor |
| | | type |
| PTPRZ1 | NM_002851 | protein tyrosine phosphatase, receptor-type, |
| PTS | NM_000317 | 6-pyruvoyltetrahydropterin synthase |
| PUNC | NM_004884 | putative neuronal cell adhesion molecule |
| PVRL4 | NM_030916 | poliovirus receptor-related 4 |
| QKI | NM_006775 | quaking homolog, KH domain RNA binding |
| | | isoform |
| QTRTD1 | NM_024638 | queuine tRNA-ribosyltransferase domain |
| R3HDM2 | NM_014925 | hypothetical protein LOC22864 |
| RAB11FIP2 | NM_014904 | RAB11 family interacting protein 2 (class I) |
| RAB11FIP5 | NM_015470 | RAB11 family interacting protein 5 (class I) |
| RAB12 | NM_001025300 | RAB12, member RAS oncogene family |
| RAB15 | NM_198686 | Ras-related protein Rab-15 |
| RAB18 | NM_021252 | RAB18, member RAS oncogene family |
| RAB22A | NM_020673 | RAS-related protein RAB-22A |
| RAB27A | NM_004580 | Ras-related protein Rab-27A |
| RAB33B | NM_031296 | RAB33B, member RAS oncogene family |
| RAB34 | NM_031934 | RAB39 |
| RAB37 | NM_001006637 | RAB37, member RAS oncogene family |
| | | isoform 1 |
| RAB39B | NM_171998 | RAB39B, member RAS oncogene family |
| RAB5A | NM_004162 | RAB5A, member RAS oncogene family |
| RAB6IP1 | NM_015213 | RAB6 interacting protein 1 |
| RAB7 | NM_004637 | RAB7, member RAS oncogene family |
| RAB8B | NM_016530 | RAB8B, member RAS oncogene family |
| RABEP1 | NM_004703 | rabaptin, RAB GTPase binding effector |
| | | protein 1 |
| RABIF | NM_002871 | RAB-interacting factor |
| RABL3 | NM_173825 | RAB, member of RAS oncogene family-like 3 |
| RAFTLIN | NM_015150 | raft-linking protein |
| RAG1 | NM_000448 | recombination activating gene 1 |
| RALBP1 | NM_006788 | ralA binding protein 1 |
| RAN | NM_006325 | ras-related nuclear protein |
| RANBP10 | NM_020850 | RAN binding protein 10 |
| RANBP6 | NM_012416 | RAN binding protein 6 |
| RANBP9 | NM_005493 | RAN binding protein 9 |
| RAP1B | NM_001010942 | RAP1B, member of RAS oncogene family |
| RAP2A | NM_021033 | RAP2A, member of RAS oncogene family |
| RAP2C | NM_021183 | RAP2C, member of RAS oncogene family |
| RAPGEF1 | NM_005312 | guanine nucleotide-releasing factor 2 isoform a |
| RASA3 | NM_007368 | RAS p21 protein activator 3 |
| RASGEF1B | NM_152545 | RasGEF domain family, member 1B |
| RASGRP1 | NM_005739 | RAS guanyl releasing protein 1 |
| RASSF6 | NM_177532 | Ras association (RalGDS/AF-6) domain |
| | | family 6 |
| RBAK | NM_021163 | RB-associated KRAB repressor |
| RBBP9 | NM_006606 | retinoblastoma binding protein 9 |
| RBM12B | NM_203390 | hypothetical protein LOC389677 |
| RBM33 | NM_001008408 | hypothetical protein LOC155435 |
| RBM35A | NM_017697 | hypothetical protein LOC54845 isoform 1 |
| RBM8A | NM_005105 | RNA binding motif protein 8A |
| RCN1 | NM_002901 | reticulocalbin 1 precursor |
| RDHE2 | NM_138969 | epidermal retinal dehydrogenase 2 |
| RDX | NM_002906 | radixin |
| RECK | NM_021111 | RECK protein precursor |
| RECQL5 | NM_001003716 | RecQ protein-like 5 isoform 3 |
| REEP1 | NM_022912 | receptor expression enhancing protein 1 |
| REEP5 | NM_005669 | receptor accessory protein 5 |
| RELN | NM_005045 | reelin isoform a |
| REV1L | NM_016316 | REV1-like isoform 1 |
| REV3L | NM_002912 | REV3-like, catalytic subunit of DNA |
| | | polymerase |
| RFC3 | NM_002915 | replication factor C 3 isoform 1 |
| RFP2 | NM_001007278 | ret finger protein 2 isoform 2 |
| RFPL3 | NM_006604 | ret finger protein-like 3 |
| RFT1 | NM_052859 | hypothetical protein LOC91869 |
| RGL1 | NM_015149 | ral guanine nucleotide dissociation |
| RGS5 | NM_003617 | regulator of G-protein signalling 5 |
| RHOA | NM_001664 | ras homolog gene family, member A |
| RHOT1 | NM_001033566 | ras homolog gene family, member T1 isoform 2 |
| RIMS3 | NM_014747 | regulating synaptic membrane exocytosis 3 |
| RIPK2 | NM_003821 | receptor-interacting serine-threonine kinase 2 |
| RIPK4 | NM_020639 | ankyrin repeat domain 3 |
| RIPK5 | NM_015375 | receptor interacting protein kinase 5 isoform 1 |
| RKHD2 | NM_016626 | ring finger and KH domain containing 2 |
| RLF | NM_012421 | rearranged L-myc fusion sequence |
| RNASEL | NM_021133 | ribonuclease L |
| RNASEN | NM_013235 | ribonuclease III, nuclear |
| RND3 | NM_005168 | ras homolog gene family, member E |
| RNF113B | NM_178861 | ring finger protein 113B |
| RNF13 | NM_007282 | ring finger protein 13 isoform 1 |
| RNF139 | NM_007218 | ring finger protein 139 |
| RNF150 | NM_020724 | ring finger protein 150 |
| RNF186 | NM_019062 | ring finger protein 186 |
| RNF19 | NM_015435 | ring finger protein 19 |
| RNF2 | NM_007212 | ring finger protein 2 |
| RNF39 | NM_025236 | HZFw1 protein isoform 1 |
| RNF8 | NM_003958 | ring finger protein 8 isoform 1 |
| RNGTT | NM_003800 | RNA guanylyltransferase and 5′-phosphatase |
| ROCK2 | NM_004850 | Rho-associated, coiled-coil containing protein |
| ROD1 | NM_005156 | ROD1 regulator of differentiation 1 |
| ROR2 | NM_004560 | receptor tyrosine kinase-like orphan receptor 2 |
| RP2 | NM_006915 | XRP2 protein |
| RPGR | NM_000328 | retinitis pigmentosa GTPase regulator isoform A |
| RPL28 | NM_000991 | ribosomal protein L28 |
| RPS23 | NM_001025 | ribosomal protein S23 |
| RPS6KA2 | NM_001006932 | ribosomal protein S6 kinase, 90 kDa, |
| | | polypeptide |
| RPS6KA3 | NM_004586 | ribosomal protein S6 kinase, 90 kDa, |
| | | polypeptide |
| RPS6KB1 | NM_003161 | ribosomal protein S6 kinase, 70 kDa, |
| | | polypeptide |
| RRM2B | NM_015713 | ribonucleotide reductase M2 B (TP53 |
| | | inducible) |
| RSAD2 | NM_080657 | radical S-adenosyl methionine domain |
| | | containing |
| RSBN1L | NM_198467 | round spermatid basic protein 1-like |
| RSN | NM_002956 | restin isoform a |
| RSU1 | NM_012425 | ras suppressor protein 1 isoform 1 |
| RTF1 | NM_015138 | Paf1/RNA polymerase II complex component |
| RUNDC2A | NM_032167 | RUN domain containing 2A |
| RUNX1 | NM_001001890 | runt-related transcription factor 1 isoform b |
| RUSC2 | NM_014806 | RUN and SH3 domain containing 2 |
| RXRA | NM_002957 | retinoid X receptor, alpha |
| RY1 | NM_006857 | putative nucleic acid binding protein RY-1 |
| S100A7L1 | NM_176823 | S100 calcium binding protein A7-like 1 |
| S100PBP | NM_022753 | S100P binding protein Riken isoform a |
| SAE1 | NM_005500 | SUMO-1 activating enzyme subunit 1 |
| SAMD9 | NM_017654 | sterile alpha motif domain containing 9 |
| SAPS3 | NM_018312 | SAPS domain family, member 3 |
| SARM1 | NM_015077 | sterile alpha and TIR motif containing 1 |
| SART1 | NM_005146 | squamous cell carcinoma antigen recognized |
| | | by T |
| SASH1 | NM_015278 | SAM and SH3 domain containing 1 |
| SBF1 | NM_002972 | SET binding factor 1 isoform a |
| SCAMP1 | NM_004866 | secretory carrier membrane protein 1 isoform 1 |
| SCARA3 | NM_016240 | scavenger receptor class A, member 3 isoform 1 |
| SCD | NM_005063 | stearoyl-CoA desaturase |
| SCML1 | NM_006746 | sex comb on midleg-like 1 isoform b |
| SCML4 | NM_198081 | sex comb on midleg-like 4 |
| SCN3A | NM_006922 | sodium channel, voltage-gated, type III, alpha |
| SCN5A | NM_000335 | voltage-gated sodium channel type V alpha |
| SCO1 | NM_004589 | cytochrome oxidase deficient homolog 1 |
| SCOC | NM_032547 | short coiled-coil protein |
| SCP2 | NM_001007099 | sterol carrier protein 2 isoform 1 precursor |
| SCRN3 | NM_024583 | secernin 3 |
| SCRT2 | NM_033129 | scratch 2 protein |
| SEC23A | NM_006364 | SEC23-related protein A |
| SEC63 | NM_007214 | SEC63-like protein |
| SEH1L | NM_031216 | sec13-like protein isoform 2 |
| SEL1L | NM_005065 | sel-1 suppressor of lin-12-like |
| SEMA3F | NM_004186 | semaphorin 3F |
| SEMA5A | NM_003966 | semaphorin 5A |
| SEMA6D | NM_020858 | semaphorin 6D isoform 1 precursor |
| SENP7 | NM_020654 | sentrin/SUMO-specific protease 7 |
| SEPT11 | NM_018243 | septin 11 |
| SEPT4 | NM_080417 | septin 4 isoform 4 |
| SERF1A | NM_021967 | small EDRK-rich factor 1A, telomeric |
| SERF1B | NM_022978 | small EDRK-rich factor 1B, centromeric |
| SERF2 | NM_001018108 | small EDRK-rich factor 2 |
| SERINC1 | NM_020755 | tumor differentially expressed 2 |
| SERPINH1 | NM_001235 | serine (or cysteine) proteinase inhibitor, clade |
| SESN1 | NM_014454 | sestrin 1 |
| SESN2 | NM_031459 | sestrin 2 |
| SETBP1 | NM_015559 | SET binding protein 1 |
| SETX | NM_015046 | senataxin |
| SF3A3 | NM_006802 | splicing factor 3a, subunit 3 |
| SFRS1 | NM_006924 | splicing factor, arginine/serine-rich 1 |
| SFRS2 | NM_003016 | splicing factor, arginine/serine-rich 2 |
| SFRS6 | NM_006275 | arginine/serine-rich splicing factor 6 |
| SFTPA2 | NM_006926 | surfactant, pulmonary-associated protein A2 |
| SGCB | NM_000232 | sarcoglycan, beta (43 kDa dystrophin- |
| | | associated |
| SGCD | NM_000337 | delta-sarcoglycan isoform 1 |
| SGCE | NM_003919 | sarcoglycan, epsilon |
| SGEF | NM_015595 | Src homology 3 domain-containing guanine |
| SGIP1 | NM_032291 | SH3-domain GRB2-like (endophilin) |
| | | interacting |
| SH2D1B | NM_053282 | SH2 domain containing 1B |
| SH3BGRL2 | NM_031469 | SH3 domain binding glutamic acid-rich |
| | | protein |
| SH3BP2 | NM_003023 | SH3-domain binding protein 2 |
| SH3BP5 | NM_001018009 | SH3-domain binding protein 5 (BTK- |
| | | associated) |
| SH3PXD2A | NM_014631 | SH3 multiple domains 1 |
| SHC1 | NM_003029 | SHC (Src homology 2 domain containing) |
| SHC4 | NM_203349 | rai-like protein |
| SHCBP1 | NM_024745 | SHC SH2-domain binding protein 1 |
| SHE | NM_001010846 | Src homology 2 domain containing E |
| SHOC2 | NM_007373 | soc-2 suppressor of clear homolog |
| SIAH1 | NM_001006610 | seven in absentia homolog 1 isoform b |
| SIN3B | NM_015260 | SIN3 homolog B, transcription regulator |
| SIRPB1 | NM_006065 | signal-regulatory protein beta 1 precursor |
| SIRT1 | NM_012238 | sirtuin 1 |
| SKP1A | NM_006930 | S-phase kinase-associated protein 1A isoform a |
| SLAMF8 | NM_020125 | B lymphocyte activator macrophage expressed |
| SLC10A2 | NM_000452 | solute carrier family 10 (sodium/bile acid |
| SLC12A5 | NM_020708 | solute carrier family 12 member 5 |
| SLC13A3 | NM_001011554 | solute carrier family 13 member 3 isoform b |
| SLC14A1 | NM_015865 | RACH1 |
| SLC16A12 | NM_213606 | solute carrier family 16 (monocarboxylic acid |
| SLC16A14 | NM_152527 | solute carrier family 16 (monocarboxylic acid |
| SLC19A3 | NM_025243 | solute carrier family 19, member 3 |
| SLC1A1 | NM_004170 | solute carrier family 1, member 1 |
| SLC1A2 | NM_004171 | solute carrier family 1, member 2 |
| SLC23A2 | NM_005116 | solute carrier family 23 (nucleobase |
| SLC24A1 | NM_004727 | solute carrier family 24 |
| SLC24A4 | NM_153646 | solute carrier family 24 member 4 isoform 1 |
| SLC25A27 | NM_004277 | solute carrier family 25, member 27 |
| SLC25A3 | NM_213612 | solute carrier family 25 member 3 isoform c |
| SLC25A36 | NM_018155 | solute carrier family 25, member 36 |
| SLC26A2 | NM_000112 | solute carrier family 26 member 2 |
| SLC26A7 | NM_052832 | solute carrier family 26, member 7 isoform a |
| SLC2A10 | NM_030777 | solute carrier family 2 member 10 |
| SLC2A2 | NM_000340 | solute carrier family 2 (facilitated glucose |
| SLC30A7 | NM_133496 | zinc transporter like 2 |
| SLC31A1 | NM_001859 | solute carrier family 31 (copper transporters), |
| SLC35A1 | NM_006416 | solute carrier family 35 (CMP-sialic acid |
| SLC35A2 | NM_005660 | solute carrier family 35 member A2 isoform a |
| SLC35B4 | NM_032826 | solute carrier family 35, member B4 |
| SLC38A2 | NM_018976 | solute carrier family 38, member 2 |
| SLC38A4 | NM_018018 | solute carrier family 38, member 4 |
| SLC39A10 | NM_020342 | solute carrier family 39 (zinc transporter), |
| SLC39A14 | NM_015359 | solute carrier family 39 (zinc transporter), |
| SLC39A8 | NM_022154 | solute carrier family 39 (zinc transporter), |
| SLC41A1 | NM_173854 | solute carrier family 41 member 1 |
| SLC4A4 | NM_003759 | solute carrier family 4, sodium bicarbonate |
| SLC4A7 | NM_003615 | solute carrier family 4, sodium bicarbonate |
| SLC6A1 | NM_003042 | solute carrier family 6 (neurotransmitter |
| SLC6A17 | NM_001010898 | solute carrier family 6, member 17 |
| SLC6A6 | NM_003043 | solute carrier family 6 (neurotransmitter |
| SLC7A11 | NM_014331 | solute carrier family 7, (cationic amino acid |
| SLC9A2 | NM_003048 | solute carrier family 9 (sodium/hydrogen |
| SLC9A3R1 | NM_004252 | solute carrier family 9 (sodium/hydrogen |
| SLCO1C1 | NM_017435 | solute carrier organic anion transporter family, |
| SLCO4C1 | NM_180991 | solute carrier organic anion transporter family, |
| SLFN12 | NM_018042 | schlafen family member 12 |
| SLK | NM_014720 | serine/threonine kinase 2 |
| SLTM | NM_001013843 | modulator of estrogen induced transcription |
| SMAD2 | NM_001003652 | Sma- and Mad-related protein 2 |
| SMAD3 | NM_005902 | MAD, mothers against decapentaplegic |
| | | homolog 3 |
| SMAD4 | NM_005359 | MAD, mothers against decapentaplegic |
| | | homolog 4 |
| SMAD5 | NM_001001419 | SMAD, mothers against DPP homolog 5 |
| SMAD7 | NM_005904 | MAD, mothers against decapentaplegic |
| | | homolog 7 |
| SMAD9 | NM_005905 | MAD, mothers against decapentaplegic |
| | | homolog 9 |
| SMARCD1 | NM_003076 | SWI/SNF-related matrix-associated |
| SMG7 | NM_014837 | SMG-7 homolog isoform 3 |
| SMPX | NM_014332 | small muscle protein, X-linked |
| SMURF1 | NM_020429 | Smad ubiquitination regulatory factor 1 |
| | | isoform |
| SMURF2 | NM_022739 | SMAD specific E3 ubiquitin protein ligase 2 |
| SNAI2 | NM_003068 | snail 2 |
| SNAP25 | NM_003081 | synaptosomal-associated protein 25 isoform |
| SNAPC1 | NM_003082 | small nuclear RNA activating complex, |
| SNRPE | NM_003094 | small nuclear ribonucleoprotein polypeptide E |
| SNURF | NM_005678 | SNRPN upstream reading frame protein |
| SNX1 | NM_003099 | sorting nexin 1 isoform a |
| SNX10 | NM_013322 | sorting nexin 10 |
| SNX16 | NM_022133 | sorting nexin 16 isoform a |
| SOAT1 | NM_003101 | sterol O-acyltransferase (acyl-Coenzyme A: |
| SOCS3 | NM_003955 | suppressor of cytokine signaling 3 |
| SOCS4 | NM_080867 | suppressor of cytokine signaling 4 |
| SORCS1 | NM_001013031 | SORCS receptor 1 isoform b |
| SORCS3 | NM_014978 | VPS10 domain receptor protein SORCS 3 |
| SORT1 | NM_002959 | sortilin 1 preproprotein |
| SOX15 | NM_006942 | SRY-box 15 |
| SP4 | NM_003112 | Sp4 transcription factor |
| SPAST | NM_014946 | spastin isoform 1 |
| SPATA2 | NM_006038 | spermatogenesis associated 2 |
| SPATA8 | NM_173499 | hypothetical protein LOC145946 |
| SPDYA | NM_001008779 | speedy homolog 1 isoform 1 |
| SPFH1 | NM_006459 | SPFH domain family, member 1 |
| SPFH2 | NM_001003790 | SPFH domain family, member 2 isoform 2 |
| SPRED1 | NM_152594 | sprouty-related protein 1 with EVH-1 domain |
| SPRY3 | NM_005840 | sprouty homolog 3 |
| SPTB | NM_001024858 | spectrin beta isoform a |
| SPTLC2 | NM_004863 | serine palmitoyltransferase, long chain base |
| SRF | NM_003131 | serum response factor (c-fos serum response |
| SRGAP3 | NM_001033116 | SLIT-ROBO Rho GTPase activating protein 3 |
| SRP72 | NM_006947 | signal recognition particle 72 kDa |
| SSFA2 | NM_006751 | sperm specific antigen 2 |
| SSR3 | NM_007107 | signal sequence receptor gamma subunit |
| ST3GAL5 | NM_003896 | sialyltransferase 9 |
| ST6GALNAC3 | NM_152996 | ST6 |
| ST6GALNAC5 | NM_030965 | sialyltransferase 7E |
| ST7 | NM_021908 | suppression of tumorigenicity 7 isoform b |
| ST8SIA2 | NM_006011 | ST8 alpha-N-acetyl-neuraminide |
| STAC | NM_003149 | SH3 and cysteine rich domain |
| STAM2 | NM_005843 | signal transducing adaptor molecule 2 |
| STARD13 | NM_052851 | START domain containing 13 isoform gamma |
| STAT5A | NM_003152 | signal transducer and activator of transcription |
| STC2 | NM_003714 | stanniocalcin 2 precursor |
| STCH | NM_006948 | stress 70 protein chaperone, |
| STEAP4 | NM_024636 | tumor necrosis factor, alpha-induced protein 9 |
| STK25 | NM_006374 | serine/threonine kinase 25 |
| STK38L | NM_015000 | serine/threonine kinase 38 like |
| STRN3 | NM_014574 | nuclear autoantigen |
| STX16 | NM_001001433 | syntaxin 16 isoform a |
| STX1A | NM_004603 | syntaxin 1A (brain) |
| STX1B2 | NM_052874 | syntaxin 1B2 |
| STYK1 | NM_018423 | serine/threonine/tyrosine kinase 1 |
| SUFU | NM_016169 | suppressor of fused |
| SUGT1 | NM_006704 | suppressor of G2 allele of SKP1 |
| SUHW4 | NM_001002844 | suppressor of hairy wing homolog 4 isoform 3 |
| SULF1 | NM_015170 | sulfatase 1 |
| SUMF2 | NM_015411 | sulfatase modifying factor 2 |
| SURF1 | NM_003172 | surfeit 1 |
| SURF4 | NM_033161 | surfeit 4 |
| SUZ12 | NM_015355 | joined to JAZF1 |
| SVH | NM_031905 | SVH protein |
| SYDE1 | NM_033025 | synapse defective 1, Rho GTPase, homolog 1 |
| SYNJ1 | NM_003895 | synaptojanin 1 isoform a |
| SYT1 | NM_005639 | synaptotagmin I |
| SYT10 | NM_198992 | synaptotagmin 10 |
| SYT15 | NM_031912 | synaptotagmin XV isoform a |
| SYVN1 | NM_032431 | synoviolin 1 isoform a |
| TACC1 | NM_006283 | transforming, acidic coiled-coil containing |
| TAF11 | NM_005643 | TBP-associated factor 11 |
| TAF12 | NM_005644 | TAF12 RNA polymerase II, TATA box |
| | | binding |
| TAF5L | NM_001025247 | PCAF associated factor 65 beta isoform b |
| TAF9B | NM_015975 | transcription associated factor 9B |
| TAOK3 | NM_016281 | TAO kinase 3 |
| TAP2 | NM_000544 | transporter 2, ATP-binding cassette, sub- |
| | | family |
| TAPBP | NM_003190 | tapasin isoform 1 precursor |
| TARDBP | NM_007375 | TAR DNA binding protein |
| TBC1D13 | NM_018201 | TBC1 domain family, member 13 |
| TBC1D15 | NM_022771 | TBC1 domain family, member 15 |
| TBC1D22B | NM_017772 | TBC1 domain family, member 22B |
| TBC1D9 | NM_015130 | hypothetical protein LOC23158 |
| TBK1 | NM_013254 | TANK-binding kinase 1 |
| TBL1X | NM_005647 | transducin beta-like 1X |
| TBL1XR1 | NM_024665 | nuclear receptor co-repressor/HDAC3 |
| | | complex |
| TBP | NM_003194 | TATA box binding protein |
| TBX22 | NM_016954 | T-box 22 |
| TBX4 | NM_018488 | T-box 4 |
| TBX5 | NM_000192 | T-box 5 isoform 1 |
| TCEB1 | NM_005648 | elongin C |
| TCF12 | NM_003205 | transcription factor 12 isoform b |
| TCF2 | NM_000458 | transcription factor 2 isoform a |
| TCF8 | NM_030751 | transcription factor 8 (represses interleukin 2 |
| TCP1 | NM_001008897 | T-complex protein 1 isoform b |
| TCP11L1 | NM_018393 | hypothetical protein LOC55346 |
| TCP11L2 | NM_152772 | hypothetical protein LOC255394 |
| TDP1 | NM_001008744 | tyrosyl-DNA phosphodiesterase 1 |
| TEAD1 | NM_021961 | TEA domain family member 1 |
| TEC | NM_003215 | tec protein tyrosine kinase |
| TERF1 | NM_003218 | telomeric repeat binding factor 1 isoform 2 |
| TERF2 | NM_005652 | telomeric repeat binding factor 2 |
| TES | NM_015641 | testin isoform 1 |
| TEX9 | NM_198524 | testis expressed sequence 9 |
| TFE3 | NM_006521 | transcription factor binding to IGHM enhancer 3 |
| TFEC | NM_001018058 | transcription factor EC isoform b |
| TGFBI | NM_000358 | transforming growth factor, beta-induced, |
| | | 68 kDa |
| TGFBR1 | NM_004612 | transforming growth factor, beta receptor I |
| THAP1 | NM_018105 | THAP domain containing, apoptosis |
| | | associated |
| THAP2 | NM_031435 | THAP domain containing, apoptosis |
| | | associated |
| THRAP1 | NM_005121 | thyroid hormone receptor associated protein 1 |
| THRAP2 | NM_015335 | thyroid hormone receptor associated protein 2 |
| THRAP6 | NM_080651 | TRAP/Mediator complex component TRAP25 |
| THUMPD3 | NM_015453 | THUMP domain containing 3 |
| TIFA | NM_052864 | TRAF-interacting protein with a |
| TIMELESS | NM_003920 | timeless homolog |
| TIMM10 | NM_012456 | translocase of inner mitochondrial membrane |
| | | 10 |
| TIMP2 | NM_003255 | tissue inhibitor of metalloproteinase 2 |
| TIPARP | NM_015508 | TCDD-inducible poly(ADP-ribose) |
| | | polymerase |
| TIPRL | NM_152902 | TIP41, TOR signalling pathway regulator-like |
| TLL1 | NM_012464 | tolloid-like 1 |
| TLL2 | NM_012465 | tolloid-like 2 |
| TLN1 | NM_006289 | talin 1 |
| TLN2 | NM_015059 | talin 2 |
| TLOC1 | NM_003262 | translocation protein 1 |
| TM7SF3 | NM_016551 | transmembrane 7 superfamily member 3 |
| TMCC1 | NM_001017395 | transmembrane and coiled-coil domains 1 |
| | | isoform |
| TMED10 | NM_006827 | transmembrane trafficking protein |
| TMED7 | NM_181836 | transmembrane emp24 protein transport |
| | | domain |
| TMEFF2 | NM_016192 | transmembrane protein with EGF-like and two |
| TMEM1 | NM_003274 | transmembrane protein 1 isoform a |
| TMEM100 | NM_018286 | hypothetical protein LOC55273 |
| TMEM106B | NM_018374 | hypothetical protein LOC54664 |
| TMEM113 | NM_025222 | hypothetical protein PRO2730 |
| TMEM119 | NM_181724 | hypothetical protein LOC338773 |
| TMEM123 | NM_052932 | pro-oncosis receptor inducing membrane |
| | | injury |
| TMEM16F | NM_001025356 | transmembrane protein 16F |
| TMEM16H | NM_020959 | hypothetical protein LOC57719 |
| TMEM25 | NM_032780 | transmembrane protein 25 |
| TMEM26 | NM_178505 | transmembrane protein 26 |
| TMEM33 | NM_018126 | transmembrane protein 33 |
| TMEM43 | NM_024334 | transmembrane protein 43 |
| TMEM46 | NM_001007538 | transmembrane protein 46 |
| TMEM47 | NM_031442 | transmembrane 4 superfamily member 10 |
| TMEM55B | NM_144568 | transmembrane protein 55B |
| TMEM70 | NM_017866 | hypothetical protein LOC54968 isoform a |
| TMEM87B | NM_032824 | hypothetical protein LOC84910 |
| TMOD1 | NM_003275 | tropomodulin 1 |
| TMPRSS11E | NM_014058 | transmembrane protease, serine 11E |
| TMTC1 | NM_175861 | ARG99 protein |
| TMTC3 | NM_181783 | hypothetical protein LOC160418 |
| TNFAIP1 | NM_021137 | tumor necrosis factor, alpha-induced protein 1 |
| TNFRSF10B | NM_003842 | tumor necrosis factor receptor superfamily, |
| TNFSF4 | NM_003326 | tumor necrosis factor (ligand) superfamily, |
| TNFSF8 | NM_001244 | tumor necrosis factor (ligand) superfamily, |
| TNKS2 | NM_025235 | tankyrase, TRF1-interacting ankyrin-related |
| TNNI1 | NM_003281 | troponin I, skeletal, slow |
| TNRC15 | NM_015575 | trinucleotide repeat containing 15 |
| TNS3 | NM_022748 | tensin-like SH2 domain containing 1 |
| TOB1 | NM_005749 | transducer of ERBB2, 1 |
| TOMM70A | NM_014820 | translocase of outer mitochondrial membrane |
| | | 70 |
| TOPORS | NM_005802 | topoisomerase I binding, arginine/serine-rich |
| TOR1AIP1 | NM_015602 | lamina-associated polypeptide 1B |
| TP53INP1 | NM_033285 | tumor protein p53 inducible nuclear protein 1 |
| TP53INP2 | NM_021202 | tumor protein p53 inducible nuclear protein 2 |
| TP53TG3 | NM_016212 | hypothetical protein LOC24150 |
| TPARL | NM_018475 | TPA regulated locus |
| TPD52 | NM_001025252 | tumor protein D52 isoform 1 |
| TPD52L1 | NM_001003395 | tumor protein D52-like 1 isoform 2 |
| TPK1 | NM_022445 | thiamin pyrophosphokinase 1 |
| TRAF6 | NM_004620 | TNF receptor-associated factor 6 |
| TRAM1 | NM_014294 | translocating chain-associating membrane |
| TRAPPC6B | NM_177452 | trafficking protein particle complex 6B |
| TREML4 | NM_198153 | triggering receptor expressed on myeloid |
| TRFP | NM_004275 | Trf (TATA binding protein-related |
| TRHDE | NM_013381 | thyrotropin-releasing hormone degrading |
| | | enzyme |
| TRIM2 | NM_015271 | tripartite motif-containing 2 |
| TRIM23 | NM_001656 | ADP-ribosylation factor domain protein 1 |
| | | isoform |
| TRIM33 | NM_015906 | tripartite motif-containing 33 protein isoform |
| TRIM4 | NM_033017 | tripartite motif protein TRIM4 isoform alpha |
| TRIM52 | NM_032765 | hypothetical protein LOC84851 |
| TRIM56 | NM_030961 | tripartite motif-containing 56 |
| TRIM62 | NM_018207 | tripartite motif-containing 62 |
| TRIM9 | NM_052978 | tripartite motif protein 9 isoform 2 |
| TRIO | NM_007118 | triple functional domain (PTPRF interacting) |
| TRMT5 | NM_020810 | tRNA-(N1G37) methyltransferase |
| TROVE2 | NM_004600 | 60 kD Ro/SSA autoantigen |
| TRPS1 | NM_014112 | zinc finger transcription factor TRPS1 |
| TSC1 | NM_000368 | tuberous sclerosis 1 protein isoform 1 |
| TSC22D1 | NM_006022 | TSC22 domain family 1 isoform 2 |
| TSC22D2 | NM_014779 | TSC22 domain family 2 |
| TSC22D3 | NM_001015881 | TSC22 domain family, member 3 isoform 3 |
| TSGA14 | NM_018718 | testis specific, 14 |
| TSHR | NM_000369 | thyroid stimulating hormone receptor isoform 1 |
| TSPAN12 | NM_012338 | transmembrane 4 superfamily member 12 |
| TSPAN13 | NM_014399 | tetraspan NET-6 |
| TSPAN33 | NM_178562 | penumbra |
| TSSK1 | NM_032028 | testis-specific serine kinase 1 |
| TTC23 | NM_001018029 | tetratricopeptide repeat domain 23 isoform 1 |
| TTC3 | NM_001001894 | tetratricopeptide repeat domain 3 |
| TTC5 | NM_138376 | tetratricopeptide repeat domain 5 |
| TTF1 | NM_007344 | transcription termination factor, RNA |
| | | polymerase |
| TTF2 | NM_003594 | transcription termination factor, RNA |
| | | polymerase |
| TUBB | NM_178014 | tubulin, beta polypeptide |
| TUBB3 | NM_006086 | tubulin, beta, 4 |
| TUFT1 | NM_020127 | tuftelin 1 |
| TULP3 | NM_003324 | tubby like protein 3 |
| TULP4 | NM_001007466 | tubby like protein 4 isoform 2 |
| TUSC2 | NM_007275 | tumor suppressor candidate 2 |
| TWISTNB | NM_001002926 | TWIST neighbor |
| TXNDC5 | NM_022085 | thioredoxin domain containing 5 isoform 2 |
| TXNDC6 | NM_178130 | thioredoxin-like 2 |
| UBE2B | NM_003337 | ubiquitin-conjugating enzyme E2B |
| UBE2D1 | NM_003338 | ubiquitin-conjugating enzyme E2D 1 |
| UBE2N | NM_003348 | ubiquitin-conjugating enzyme E2N |
| UBE2R2 | NM_017811 | ubiquitin-conjugating enzyme UBC3B |
| UBE2W | NM_001001481 | hypothetical protein LOC55284 isoform 1 |
| UBP1 | NM_014517 | upstream binding protein 1 (LBP-1a) |
| UBQLN1 | NM_013438 | ubiquilin 1 isoform 1 |
| UBXD2 | NM_014607 | UBX domain containing 2 |
| UCHL5 | NM_015984 | ubiquitin C-terminal hydrolase UCH37 |
| ULK2 | NM_014683 | unc-51-like kinase 2 |
| UNC50 | NM_014044 | unc-50 homolog |
| USH1G | NM_173477 | Usher syndrome 1G protein |
| USH2A | NM_007123 | usherin isoform A |
| USP12 | NM_182488 | ubiquitin-specific protease 12-like 1 |
| USP15 | NM_006313 | ubiquitin specific protease 15 |
| USP18 | NM_017414 | ubiquitin specific protease 18 |
| USP25 | NM_013396 | ubiquitin specific protease 25 |
| USP33 | NM_015017 | ubiquitin specific protease 33 isoform 1 |
| USP46 | NM_022832 | ubiquitin specific protease 46 |
| USP47 | NM_017944 | ubiquitin specific protease 47 |
| USP49 | NM_018561 | ubiquitin specific protease 49 |
| USP9Y | NM_004654 | ubiquitin specific protease 9, Y-linked |
| UTY | NM_182659 | tetratricopeptide repeat protein isoform 2 |
| UXS1 | NM_025076 | UDP-glucuronate decarboxylase 1 |
| VANGL2 | NM_020335 | vang-like 2 (van gogh, Drosophila) |
| VASH1 | NM_014909 | vasohibin 1 |
| VDP | NM_003715 | vesicle docking protein p115 |
| VGLL2 | NM_153453 | vestigial-like 2 isoform 2 |
| VGLL3 | NM_016206 | colon carcinoma related protein |
| VLDLR | NM_001018056 | very low density lipoprotein receptor isoform b |
| VNN2 | NM_004665 | vanin 2 isoform 1 precursor |
| VPS13C | NM_017684 | vacuolar protein sorting 13C protein isoform |
| | | 1A |
| VTI1A | NM_145206 | SNARE Vti1a-beta protein |
| WAPAL | NM_015045 | wings apart-like homolog |
| WASF1 | NM_001024934 | Wiskott-Aldrich syndrome protein family |
| | | member |
| WASF3 | NM_006646 | WAS protein family, member 3 |
| WBP1 | NM_012477 | WW domain binding protein 1 |
| WBP4 | NM_007187 | WW domain-containing binding protein 4 |
| WBSCR1 | NM_022170 | eukaryotic translation initiation factor 4H |
| WDFY3 | NM_014991 | WD repeat and FYVE domain containing 3 |
| | | isoform |
| WDHD1 | NM_001008396 | WD repeat and HMG-box DNA binding |
| | | protein 1 |
| WDR21C | NM_152418 | hypothetical protein LOC138009 |
| WDR35 | NM_001006657 | WD repeat domain 35 isoform 1 |
| WDR42A | NM_015726 | H326 |
| WDR45L | NM_019613 | WDR45-like |
| WDR68 | NM_005828 | WD-repeat protein |
| WHSC1 | NM_133334 | Wolf-Hirschhorn syndrome candidate 1 |
| | | protein |
| WIF1 | NM_007191 | Wnt inhibitory factor-1 precursor |
| WIPI2 | NM_001033518 | hypothetical protein LOC26100 isoform c |
| WNT1 | NM_005430 | wingless-type MMTV integration site family, |
| WNT16 | NM_016087 | wingless-type MMTV integration site family, |
| WNT4 | NM_030761 | wingless-type MMTV integration site family, |
| WRB | NM_004627 | tryptophan rich basic protein |
| WSB1 | NM_015626 | WD repeat and SOCS box-containing 1 |
| | | isoform 1 |
| WWC1 | NM_015238 | KIBRA protein |
| WWP2 | NM_199423 | WW domain containing E3 ubiquitin protein |
| | | ligase |
| XG | NM_175569 | XG glycoprotein precursor |
| XKR3 | NM_175878 | X Kell blood group precursor-related family, |
| XKR8 | NM_018053 | X Kell blood group precursor-related family, |
| XKRY | NM_004677 | XK, Kell blood group complex subunit- |
| | | related, |
| XKRY2 | NM_001002906 | XK, Kell blood group complex subunit- |
| | | related, |
| XPO4 | NM_022459 | exportin 4 |
| YEATS4 | NM_006530 | glioma-amplified sequence-41 |
| YES1 | NM_005433 | viral oncogene yes-1 homolog 1 |
| YOD1 | NM_018566 | hypothetical protein LOC55432 |
| YPEL2 | NM_001005404 | yippee-like 2 |
| YWHAG | NM_012479 | tyrosine 3-monooxygenase/tryptophan |
| YWHAQ | NM_006826 | tyrosine 3/tryptophan 5-monooxygenase |
| ZA20D2 | NM_006007 | zinc finger protein 216 |
| ZAK | NM_133646 | MLK-related kinase isoform 2 |
| ZBTB24 | NM_014797 | zinc finger and BTB domain containing 24 |
| ZBTB5 | NM_014872 | zinc finger and BTB domain containing 5 |
| ZBTB6 | NM_006626 | zinc finger protein 482 |
| ZBTB8 | NM_144621 | zinc finger and BTB domain containing 8 |
| ZC3H11A | NM_014827 | hypothetical protein LOC9877 |
| ZC3H12B | NM_001010888 | hypothetical protein LOC340554 |
| ZC3H6 | NM_198581 | zinc finger CCCH-type domain containing 6 |
| ZCCHC14 | NM_015144 | zinc finger, CCHC domain containing 14 |
| ZDHHC11 | NM_024786 | zinc finger, DHHC domain containing 11 |
| ZDHHC17 | NM_015336 | huntingtin interacting protein 14 |
| ZFHX1B | NM_014795 | zinc finger homeobox 1b |
| ZFHX4 | NM_024721 | zinc finger homeodomain 4 |
| ZFP1 | NM_153688 | zinc finger protein 1 homolog |
| ZFP106 | NM_022473 | zinc finger protein 106 homolog |
| ZFP161 | NM_003409 | zinc finger protein 161 homolog |
| ZFP260 | NM_001012756 | zinc finger protein 260 |
| ZFP36 | NM_003407 | zinc finger protein 36, C3H type, homolog |
| ZFP41 | NM_173832 | zinc finger protein 41 homolog |
| ZFPM2 | NM_012082 | zinc finger protein, multitype 2 |
| ZFYVE20 | NM_022340 | FYVE-finger-containing Rab5 effector protein |
| ZMAT1 | NM_001011656 | zinc finger, matrin type 1 isoform 2 |
| ZNF10 | NM_015394 | zinc finger protein 10 |
| ZNF161 | NM_007146 | zinc finger protein 161 |
| ZNF181 | NM_001029997 | zinc finger protein 181 (HHZ181) |
| ZNF192 | NM_006298 | zinc finger protein 192 |
| ZNF217 | NM_006526 | zinc finger protein 217 |
| ZNF229 | NM_014518 | zinc finger protein 229 |
| ZNF26 | NM_019591 | zinc finger protein 26 (KOX 20) |
| ZNF265 | NM_005455 | zinc finger protein 265 isoform 2 |
| ZNF267 | NM_003414 | zinc finger protein 267 |
| ZNF274 | NM_016324 | zinc finger protein 274 isoform b |
| ZNF278 | NM_014323 | zinc finger protein 278 long C isoform |
| ZNF294 | NM_015565 | zinc finger protein 294 |
| ZNF295 | NM_020727 | zinc finger protein 295 |
| ZNF300 | NM_052860 | zinc finger protein 300 |
| ZNF302 | NM_001012320 | zinc finger protein 302 |
| ZNF304 | NM_020657 | zinc finger protein 304 |
| ZNF31 | NM_145238 | zinc finger protein 31 |
| ZNF320 | NM_207333 | zinc finger protein 320 |
| ZNF326 | NM_182975 | zinc finger protein 326 isoform 3 |
| ZNF336 | NM_022482 | zinc finger protein 336 |
| ZNF33A | NM_006974 | zinc finger protein 33A |
| ZNF365 | NM_014951 | zinc finger protein 365 isoform A |
| ZNF395 | NM_018660 | zinc finger protein 395 |
| ZNF406 | NM_001029939 | zinc finger protein 406 isoform TR-ZFAT |
| ZNF420 | NM_144689 | zinc finger protein 420 |
| ZNF480 | NM_144684 | zinc finger protein 480 |
| ZNF483 | NM_133464 | zinc finger protein 483 isoform a |
| ZNF498 | NM_145115 | zinc finger protein 498 |
| ZNF507 | NM_014910 | zinc finger protein 507 |
| ZNF510 | NM_014930 | zinc finger protein 510 |
| ZNF518 | NM_014803 | zinc finger protein 518 |
| ZNF526 | NM_133444 | zinc finger protein 526 |
| ZNF529 | NM_020951 | zinc finger protein 529 |
| ZNF532 | NM_018181 | zinc finger protein 532 |
| ZNF536 | NM_014717 | zinc finger protein 536 |
| ZNF566 | NM_032838 | zinc finger protein 566 |
| ZNF568 | NM_198539 | zinc finger protein 568 |
| ZNF577 | NM_032679 | zinc finger protein 577 |
| ZNF585A | NM_152655 | zinc finger protein 585A |
| ZNF596 | NM_173539 | zinc finger protein 596 |
| ZNF6 | NM_021998 | zinc finger protein 6 |
| ZNF605 | NM_183238 | zinc finger protein 605 |
| ZNF614 | NM_025040 | zinc finger protein 614 |
| ZNF616 | NM_178523 | zinc finger protein 616 |
| ZNF652 | NM_014897 | zinc finger protein 652 |
| ZNF655 | NM_001009956 | zinc finger protein 655 isoform e |
| ZNF662 | NM_207404 | zinc finger protein 662 |
| ZNF667 | NM_022103 | zinc finger protein 667 |
| ZNF673 | NM_017776 | zinc finger protein 673 |
| ZNF702 | NM_024924 | zinc finger protein 702 |
| ZNF706 | NM_016096 | HSPC038 protein |
| ZNF708 | NM_021269 | zinc finger protein 15-like 1 (KOX 8) |
| ZNF720 | NM_001004300 | zinc finger protein 720 |
| ZRANB3 | NM_032143 | zinc finger, RAN-binding domain containing 3 |
| ZSWIM4 | NM_023072 | zinc finger, SWIM domain containing 4 |
| ZXDB | NM_007157 | zinc finger, X-linked, duplicated B |

TABLE 4
hsa-miR-200 targets that exhibited altered mRNA expression levels in human
cancer cells after transfection with pre-miR-200.

| | Ref Seq | |
|---|---|---|
| Gene | (Pruitt et al., | |
| Symbol | 2005) | Description |
| AP1S2 | NM_003916 | adaptor-related protein complex 1 sigma 2 |
| ATP2A2 | NM_170665 | ATPase, Ca++ transporting, cardiac muscle, slow |
| B4GALT6 | NM_004775 | UDP-Gal:betaGlcNAc beta 1,4- |
| BDKRB2 | NM_000623 | bradykinin receptor B2 |
| C10orf56 | NM_153367 | hypothetical protein LOC219654 |
| C1orf24 | NM_052966 | niban protein isoform 2 |
| C8orf1 | NM_004337 | hypothetical protein LOC734 |
| CDCP1 | NM_022842 | CUB domain-containing protein 1 isoform 1 |
| CDH1 | NM_004360 | cadherin 1, type 1 preproprotein |
| CRTAP | NM_006371 | cartilage associated protein precursor |
| CXX1 | NM_003928 | CAAX box 1 |
| DDAH1 | NM_012137 | dimethylarginine dimethylaminohydrolase 1 |
| DNAJB6 | NM_005494 | DnaJ (Hsp40) homolog, subfamily B, member 6 |
| DNAJB9 | NM_012328 | DnaJ (Hsp40) homolog, subfamily B, member 9 |
| DZIP1 | NM_014934 | DAZ interacting protein 1 isoform 1 |
| FADS1 | NM_013402 | fatty acid desaturase 1 |
| FAS | NM_000043 | tumor necrosis factor receptor superfamily, |
| FEZ2 | NM_005102 | zygin 2 |
| FLJ11184 | NM_018352 | hypothetical protein LOC55319 |
| FLJ20232 | NM_019008 | hypothetical protein LOC54471 |
| FN1 | NM_002026 | fibronectin 1 isoform 3 preproprotein |
| FSTL1 | NM_007085 | follistatin-like 1 precursor |
| GNA13 | NM_006572 | guanine nucleotide binding protein (G protein), |
| GREM1 | NM_013372 | gremlin-1 precursor |
| HMOX1 | NM_002133 | heme oxygenase (decyclizing) 1 |
| HPS5 | NM_007216 | Hermansky-Pudlak syndrome 5 isoform b |
| IL8 | NM_000584 | interleukin 8 precursor |
| KCNMA1 | NM_002247 | large conductance calcium-activated potassium |
| KDELC1 | NM_024089 | KDEL (Lys-Asp-Glu-Leu) containing 1 |
| KLF4 | NM_004235 | kruppel-like factor 4 (gut) |
| LEPR | NM_001003679 | leptin receptor isoform 2 |
| LHFP | NM_005780 | lipoma HMGIC fusion partner |
| MARCKS | NM_002356 | myristoylated alanine-rich protein kinase C |
| MCFD2 | NM_139279 | multiple coagulation factor deficiency 2 |
| NR5A2 | NM_003822 | nuclear receptor subfamily 5, group A, member 2 |
| OSTM1 | NM_014028 | Osteopetrosis associated transmembrane protein |
| PCAF | NM_003884 | P300/CBP-associated factor |
| QKI | NM_006775 | quaking homolog, KH domain RNA binding isoform |
| RAB11FIP2 | NM_014904 | RAB11 family interacting protein 2 (class I) |
| RAFTLIN | NM_015150 | raft-linking protein |
| RASGRP1 | NM_005739 | RAS guanyl releasing protein 1 |
| RBM35A | NM_017697 | hypothetical protein LOC54845 isoform 1 |
| RECK | NM_021111 | RECK protein precursor |
| RP2 | NM_006915 | XRP2 protein |
| SCD | NM_005063 | stearoyl-CoA desaturase |
| SEC23A | NM_006364 | SEC23-related protein A |
| SHCBP1 | NM_024745 | SHC SH2-domain binding protein 1 |
| ST7 | NM_021908 | suppression of tumorigenicity 7 isoform b |
| STC2 | NM_003714 | Stanniocalcin 2 precursor |
| STYK1 | NM_018423 | serine/threonine/tyrosine kinase 1 |
| SYDE1 | NM_033025 | synapse defective 1, Rho GTPase, homolog 1 |
| TCF8 | NM_030751 | Transcription factor 8 (represses interleukin 2 |
| ZFHX1B | NM_014795 | zinc finger homeobox 1b |

The predicted gene targets are shown in Table 3. Predicted target genes of hsa-miR-200 whose mRNA expression levels are affected by hsa-miR-200 represent particularly useful candidates for cancer therapy and therapy of other diseases through manipulation of their expression levels.

Certain embodiments of the invention include determining expression of one or more marker, gene, or nucleic acid segment representative of one or more genes, by using an amplification assay, a hybridization assay, or protein assay, a variety of which are well known to one of ordinary skill in the art. In certain aspects, an amplification assay can be a quantitative amplification assay, such as quantitative RT-PCR or the like. In still further aspects, a hybridization assay can include array hybridization assays or solution hybridization assays. The nucleic acids from a sample may be labeled from the sample and/or hybridizing the labeled nucleic acid to one or more nucleic acid probes. Nucleic acids, mRNA, and/or nucleic acid probes may be coupled to a support. Such supports are well known to those of ordinary skill in the art and include, but are not limited to glass, plastic, metal, or latex. In particular aspects of the invention, the support can be planar or in the form of a bead or other geometric shapes or configurations known in the art. Proteins are typically assayed by immunoblotting, chromatography, or mass spectrometry or other methods known to those of ordinary skill in the art.

The present invention also concerns kits containing compositions of the invention or compositions to implement methods of the invention. In some embodiments, kits can be used to evaluate one or more marker molecules, and/or express one or more miRNA or miRNA inhibitor. In certain embodiments, a kit contains, contains at least or contains at most 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 100, 150, 200 or more probes, recombinant nucleic acid, or synthetic nucleic acid molecules related to the markers to be assessed or an miRNA or miRNA inhibitor to be expressed or modulated, and may include any range or combination derivable therein. Kits may comprise components, which may be individually packaged or placed in a container, such as a tube, bottle, vial, syringe, or other suitable container means. Individual components may also be provided in a kit in concentrated amounts; in some embodiments, a component is provided individually in the same concentration as it would be in a solution with other components. Concentrations of components may be provided as 1×, 2×, 5×, 10×, or 20× or more. Kits for using probes, synthetic nucleic acids, recombinant nucleic acids, or non-synthetic nucleic acids of the invention for therapeutic, prognostic, or diagnostic applications are included as part of the invention. Specifically contemplated are any such molecules corresponding to any miRNA reported to influence biological activity or expression of one or more marker gene or gene pathway described herein. In certain aspects, negative and/or positive controls are included in some kit embodiments. The control molecules can be used to verify transfection efficiency and/or control for transfection-induced changes in cells.

Certain embodiments are directed to a kit for assessment of a pathological condition or the risk of developing a pathological condition in a patient by nucleic acid profiling of a sample comprising, in suitable container means, two or more nucleic acid hybridization or amplification reagents. The kit can comprise reagents for labeling nucleic acids in a sample and/or nucleic acid hybridization reagents. The hybridization reagents typically comprise hybridization probes. Amplification reagents include, but are not limited to amplification primers, reagents, and enzymes.

In some embodiments of the invention, an expression profile is generated by steps that include: (a) labeling nucleic acid in the sample; (b) hybridizing the nucleic acid to a number of probes, or amplifying a number of nucleic acids, and (c) determining and/or quantitating nucleic acid hybridization to the probes or detecting and quantitating amplification products, wherein an expression profile is generated. See U.S. Provisional Patent Application 60/575,743 and the U.S. Provisional Patent Application 60/649,584, and U.S. patent application Ser. No. 11/141,707 and U.S. patent application Ser. No. 11/273,640, all of which are hereby incorporated by reference.

Methods of the invention involve diagnosing and/or assessing the prognosis of a patient based on a miRNA and/or a marker nucleic acid expression profile. In certain embodiments, the elevation or reduction in the level of expression of a particular gene or genetic pathway or set of nucleic acids in a cell is correlated with a disease state or pathological condition compared to the expression level of the same in a normal or non-pathologic cell or tissue sample. This correlation allows for diagnostic and/or prognostic methods to be carried out when the expression level of one or more nucleic acid is measured in a biological sample being assessed and then compared to the expression level of a normal or non-pathologic cell or tissue sample. It is specifically contemplated that expression profiles for patients, particularly those suspected of having or having a propensity for a particular disease or condition such as cancer, can be generated by evaluating any of or sets of the miRNAs and/or nucleic acids discussed in this application. The expression profile that is generated from the patient will be one that provides information regarding the particular disease or condition. In many embodiments, the profile is generated using nucleic acid hybridization or amplification, (e.g., array hybridization or RT-PCR). In certain aspects, an expression profile can be used in conjunction with other diagnostic and/or prognostic tests, such as histology, protein profiles in the serum and/or cytogenetic assessment.

TABLE 5
Tumor associated mRNAs altered by hsa-miR-200 having prognostic or therapeutic value for
the treatment of various malignancies.

| Gene | | Cellular | | |
|---|---|---|---|---|
| Symbol | Gene Title | Process | Cancer Type | Reference |
| AREG | Amphiregulin | signal | HCC, NSCLC, MM, PC, OC, | (Kitadai et al., 1993; Ebert et al., 1994; Solic and |
| | | transduction | CRC, PaC, GC | Davies, 1997; D'Antonio et al., 2002; Bostwick et |
| | | | | al., 2004; Ishikawa et al., 2005; Mahtouk et al., |
| | | | | 2005; Castillo et al., 2006) □ |
| CCNG1 | cyclin G1 | cell cycle | OS, BC, PC | (Skotzko et al., 1995; Reimer et al., 1999) □ |
| CTGF | CTGF/IGFBP-8 | cell | BC, GB, OepC, RMS, CRC, | (Hishikawa et al., 1999; Shimo et al., 2001; |
| | | adhesion, | PC | Koliopanos et al., 2002; Pan et al., 2002; Croci et |
| | | migration | | al., 2004; Lin et al., 2005; Yang et al., 2005) □ |
| FAS | Fas | Apoptosis | NSCLC, G, L, CRC, OepC | (Moller et al., 1994; Gratas et al., 1998; Martinez- |
| | | | | Lorenzo et al., 1998; Shinoura et al., 2000; Viard- |
| | | | | Leveugle et al., 2003) □ |
| FGFBP1 | FGF-BP | signal | SCCHN, BC, CRC, PC, PaC | (Abuharbeid et al., 2006; Tassi et al., 2006) □ |
| | | transduction | | |
| FGFR4 | FGF receptor-4 | signal | TC, BC, OC, PaC | (Jaakkola et al., 1993; Shah et al., 2002; Ezzat et |
| | | transduction | | al., 2005) □ |
| IGFBP1 | IGFBP-1 | signal | BC, CRC | (Firth and Baxter, 2002)□ |
| | | transduction | | |
| IL8 | IL-8 | signal | BC, CRC, PaC, NSCLC, PC, | (Akiba et al., 2001; Sparmann and Bar-Sagi, 2004) □ |
| | | transduction | HCC | |
| LCN2 | lipocalin 2/ | cell adhesion | PaC, CRC, HCC, BC, OC | (Bartsch and Tschesche, 1995; Furutani et al., |
| | NGAL | | | 1998; Fernandez et al., 2005; Lee et al., 2006) □ |
| LHFP | lipoma HMGIC | transcription | Li | (Petit et al., 1999)□ |
| | fusion partner | | | |
| MCL1 | Mcl-1 | apoptosis | HCC, MM, TT, CLL, ALCL, | (Krajewska et al., 1996; Kitada et al., 1998; Cho- |
| | | | BCL, PC | Vega et al., 2004; Rust et al., 2005; Sano et al., |
| | | | | 2005; Wuilleme-Toumi et al., 2005; Fleischer et |
| | | | | al., 2006; Sieghart et al., 2006)□ |
| RARRES1 | RAR responder 1 | migration, | CRC, PC | (Zhang et al., 2004; Wu et al., 2006) □ |
| | | invasion | | |
| RBL1 | p107 | cell cycle | BCL, PC, CRC, TC | (Takimoto et al., 1998; Claudio et al., 2002; Wu et |
| | | | | al., 2002; Ito et al., 2003) □ |
| SFRP4 | Secreted | signal | MT, CLL, SCCHN | (Lee et al., 2004; Liu et al., 2006; Marsit et al., |
| | frizzled-related | transduction | | 2006) □ |
| | protein 4 | | | |
| ST7 | Suppressor of | Unknown | PC, BC | (Hooi et al., 2006)□ |
| | tumorigenicity 7 | | | |
| TACSTD1 | Tumor- | cell | NSCLC, CRC | (Xi et al., 2006a; Xi et al., 2006b) □ |
| | associated | adhesion, | | |
| | calcium signal | vesicle | | |
| | transducer 1 | trafficking | | |
| TXN | thioredoxin | thioredoxin | LC, PaC, CeC, HCC | (Marks, 2006)□ |
| | (trx) | redox system | | |
| VAV3 | Vav3 | signal | PC | (Dong et al., 2006)□ |
| | | transduction | | |

Abbreviations:
ALCL, anaplastic large cell lymphoma;
BC, breast carcinoma;
BCL, B-cell lymphoma;
CeC, cervical carcinoma;
CLL, chronic lymphoblastic leukemia;
CRC, colorectal carcinoma;
G, glioma;
GB, glioblastoma;
GC, gastric carcinoma;
HCC, hepatocellular carcinoma;
L, leukemia;
LC, lung carcinoma;
Li, lipoma;
MM, multiple myeloma;
MT, mesothelioma;
NSCLC, non-small cell lung carcinoma;
OC, ovarian carcinoma;
OepC, oesophageal carcinoma;
OS, osteosarcoma;
PaC, pancreatic carcinoma;
PC, prostate carcinoma;
RMS, rhabdomyosarcoma;
SCCHN, squamous cell carcinoma of the head and neck;
TC, thyroid carcinoma;
TT, testicular tumor.

The methods can further comprise one or more of the steps including: (a) obtaining a sample from the patient, (b) isolating nucleic acids from the sample, (c) labeling the nucleic acids isolated from the sample, and (d) hybridizing the labeled nucleic acids to one or more probes. Nucleic acids of the invention include one or more nucleic acid comprising at least one segment having a sequence or complementary sequence of to a nucleic acid representative of one or more of genes or markers in Table 1, 3, 4, and/or 5.

It is contemplated that any method or composition described herein can be implemented with respect to any other method or composition described herein and that different embodiments may be combined. It is specifically contemplated that any methods and compositions discussed herein with respect to miRNA molecules, miRNA, genes, and Certain embodiments of the invention include determining expression of one or more marker, gene, or nucleic acid representative thereof, by using an amplification assay, a hybridization assay, or protein assay, a variety of which are well known to one of ordinary skill in the art. In certain aspects, an amplification assay can be a quantitative amplification assay, such as quantitative RT-PCR or the like. In still further aspects, a hybridization assay can include array hybridization assays or solution hybridization assays. The nucleic acids from a sample may be labeled from the sample and/or hybridizing the labeled nucleic acid to one or more nucleic acid probes. Nucleic acids, mRNA, and/or nucleic acid probes may be coupled to a support. Such supports are well known to those of ordinary skill in the art and include, but are not limited to glass, plastic, metal, or latex. In particular aspects of the invention, the support can be planar or in the form of a bead or other geometric shapes or configurations known in the art. Protein are typically assayed by immunoblotting, chromatography, or mass spectrometry or other methods known to those of ordinary skill in the art.

The present invention also concerns kits containing compositions of the invention or compositions to implement methods of the invention. In some embodiments, kits can be used to evaluate one or more marker molecules, and/or express one or more miRNA. In certain embodiments, a kit contains, contains at least or contains at most 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 100, 150, 200 or more probes, recombinant nucleic acid, or synthetic nucleic acid molecules related to the markers to be assessed or an miRNA to be expressed or modulated, and may include any range or combination derivable therein. Kits may comprise components, which may be individually packaged or placed in a container, such as a tube, bottle, vial, syringe, or other suitable container means. Individual components may also be provided in a kit in concentrated amounts; in some embodiments, a component is provided individually in the same concentration as it would be in a solution with other components. Concentrations of components may be provided as 1×, 2×, 5×, 10×, or 20× or more. Kits for using probes, synthetic nucleic acids, recombinant nucleic acids, or non-synthetic nucleic acids of the invention for therapeutic, prognostic, or diagnostic applications are included as part of the invention. Specifically contemplated are any such molecules corresponding to any miRNA reported to influence biological activity or expression of one or more marker gene or gene pathway described herein. In certain aspects, negative and/or positive controls are included in some kit embodiments. The control molecules can be used to verify transfection efficiency and/or control for transfection-induced changes in cells.

Certain embodiments are directed to a kit for assessment of a pathological condition or the risk of developing a pathological condition in a patient by nucleic acid profiling of a sample comprising, in suitable container means, two or more nucleic acid hybridization or amplification reagents. The kit can comprise reagents for labeling nucleic acids in a sample and/or nucleic acid hybridization reagents. The hybridization reagents typically comprise hybridization probes. Amplification reagents include, but are not limited to amplification primers, reagents, and enzymes.

In some embodiments of the invention, an expression profile is generated by steps that include: (a) labeling nucleic acid in the sample; (b) hybridizing the nucleic acid to a number of probes, or amplifying a number of nucleic acids, and (c) determining and/or quantitating nucleic acid hybridization to the probes or detecting and quantitating amplification products, wherein an expression profile is generated. See U.S. Provisional Patent Application 60/575,743 and the U.S. Provisional Patent Application 60/649,584, and U.S. patent application Ser. No. 11/141,707 and U.S. patent application Ser. No. 11/273,640, all of which are hereby incorporated by reference.

Methods of the invention involve diagnosing and/or assessing the prognosis of a patient based on an miRNA and/or a marker nucleic acid expression profile. In certain embodiments, the elevation or reduction in the level of expression of a particular gene or genetic pathway or set of nucleic acids in a cell is correlated with a disease state or pathological condition compared to the expression level of the same in a normal or non-pathologic cell or tissue sample. This correlation allows for diagnostic and/or prognostic methods to be carried out when the expression level of one or more nucleic acid is measured in a biological sample being assessed and then compared to the expression level of a normal or non-pathologic cell or tissue sample. It is specifically contemplated that expression profiles for patients, particularly those suspected of having or having a propensity for a particular disease or condition such as cancer, can be generated by evaluating any of or sets of the miRNAs and/or nucleic acids discussed in this application. The expression profile that is generated from the patient will be one that provides information regarding the particular disease or condition. In many embodiments, the profile is generated using nucleic acid hybridization or amplification, (e.g., array hybridization or RT-PCR). In certain aspects, an expression profile can be used in conjunction with other diagnostic and/or prognostic tests, such as histology, protein profiles in the serum and/or cytogenetic assessment.

The methods can further comprise one or more of the steps including: (a) obtaining a sample from the patient, (b) isolating nucleic acids from the sample, (c) labeling the nucleic acids isolated from the sample, and (d) hybridizing the labeled nucleic acids to one or more probes. Nucleic acids of the invention include one or more nucleic acid comprising at least one segment having a sequence or complementary sequence of to a nucleic acid representative of one or more of genes or markers in Table 1, 3, 4, and/or 5.

It is contemplated that any method or composition described herein can be implemented with respect to any other method or composition described herein and that different embodiments may be combined. It is specifically contemplated that any methods and compositions discussed herein with respect to miRNA molecules, miRNA, genes and nucleic acids representative of genes may be implemented with respect to synthetic nucleic acids. In some embodiments the synthetic nucleic acid is exposed to the proper conditions to allow it to become a processed or mature nucleic acid, such as a miRNA under physiological circumstances. The claims originally filed are contemplated to cover claims that are multiply dependent on any filed claim or combination of filed claims.

Also, any embodiment of the invention involving specific genes (including representative fragments there of), mRNA, or miRNAs by name is contemplated also to cover embodiments involving miRNAs whose sequences are at least 80, 81, 82, 83, 84, 85, 86, 87, 88, 89, 90, 91, 92, 93, 94, 95, 96, 97, 98, 99% identical to the mature sequence of the specified miRNA.

It will be further understood that shorthand notations are employed such that a generic description of a gene or marker thereof, or of a miRNA refers to any of its gene family members (distinguished by a number) or representative fragments thereof, unless otherwise indicated. It is understood by those of skill in the art that a “gene family” refers to a group of genes having the same coding sequence or miRNA coding sequence. Typically, miRNA members of a gene family are identified by a number following the initial designation. For example, miR-16-1 and miR-16-2 are members of the miR-16 gene family and “mir-7” refers to miR-7-1, miR-7-2 and miR-7-3. Moreover, unless otherwise indicated, a shorthand notation refers to related miRNAs (distinguished by a letter). Exceptions to these shorthand notations will be otherwise identified.

Other embodiments of the invention are discussed throughout this application. Any embodiment discussed with respect to one aspect of the invention applies to other aspects of the invention as well and vice versa. The embodiments in the Example and Detailed Description section are understood to be embodiments of the invention that are applicable to all aspects of the invention.

The terms “inhibiting,” “reducing,” or “prevention,” or any variation of these terms, when used in the claims and/or the specification includes any measurable decrease or complete inhibition to achieve a desired result.

The use of the word “a” or “an” when used in conjunction with the term “comprising” in the claims and/or the specification may mean “one,” but it is also consistent with the meaning of “one or more,” “at least one,” and “one or more than one.”

Throughout this application, the term “about” is used to indicate that a value includes the standard deviation of error for the device or method being employed to determine the value.

The use of the term “or” in the claims is used to mean “and/or” unless explicitly indicated to refer to alternatives only or the alternatives are mutually exclusive, although the disclosure supports a definition that refers to only alternatives and “and/or.”

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### DETAILED DESCRIPTION OF THE INVENTION

The present invention is directed to compositions and methods relating to the identification and characterization of genes and biological pathways related to these genes as represented by the expression of the identified genes, as well as use of miRNAs related to such, for therapeutic, prognostic, and diagnostic applications, particularly those methods and compositions related to assessing and/or identifying pathological conditions directly or indirectly related to miR-200 expression or the aberrant expression thereof.

In certain aspects, the invention is directed to methods for the assessment, analysis, and/or therapy of a cell or subject where certain genes have a reduced or increased expression (relative to normal) as a result of an increased or decreased expression of any one or a combination of miR-200 family members (including, but not limited to SEQ ID NO:1 to SEQ ID NO:108) and/or genes with an increased expression (relative to normal) as a result of decreased expression thereof. The expression profile and/or response to miR-200 expression or inhibition may be indicative of a disease or pathological condition, e.g., cancer.

Prognostic assays featuring any one or combination of the miRNAs listed or the markers listed (including nucleic acids representative thereof) could be used in assessment of a patient to determine what if any treatment regimen is justified. As with the diagnostic assays mentioned above, the absolute values that define low expression will depend on the platform used to measure the miRNA(s). The same methods described for the diagnostic assays could be used for prognostic assays.

### I. Therapeutic Methods

Embodiments of the invention concern nucleic acids that perform the activities of or inhibit endogenous miRNAs when introduced into cells. In certain aspects, nucleic acids are synthetic or non-synthetic miRNA. Sequence-specific miRNA inhibitors can be used to inhibit sequentially or in combination the activities of one or more endogenous miRNAs in cells, as well those genes and associated pathways modulated by the endogenous miRNA.

The present invention concerns, in some embodiments, short nucleic acid molecules that function as miRNAs or as inhibitors of miRNA in a cell. The term “short” refers to a length of a single polynucleotide that is 15, 16, 17, 18, 19, 20, 21, 22, 23, 24, 25, 50, 100, or 150 nucleotides or fewer, including all integers or ranges derivable there between. The nucleic acid molecules are typically synthetic. The term “synthetic” refers to a nucleic acid molecule that is not produced naturally in a cell. In certain aspects the chemical structure deviates from a naturally-occurring nucleic acid molecule, such as an endogenous precursor miRNA or miRNA molecule or complement thereof. While in some embodiments, nucleic acids of the invention do not have an entire sequence that is identical or complementary to a sequence of a naturally-occurring nucleic acid, such molecules may encompass all or part of a naturally-occurring sequence or a complement thereof. It is contemplated, however, that a synthetic nucleic acid administered to a cell may subsequently be modified or altered in the cell such that its structure or sequence is the same as non-synthetic or naturally occurring nucleic acid, such as a mature miRNA sequence. For example, a synthetic nucleic acid may have a sequence that differs from the sequence of a precursor miRNA, but that sequence may be altered once in a cell to be the same as an endogenous, processed miRNA or an inhibitor thereof. The term “isolated” means that the nucleic acid molecules of the invention are initially separated from different (in terms of sequence or structure) and unwanted nucleic acid molecules such that a population of isolated nucleic acids is at least about 90% homogenous, and may be at least about 95, 96, 97, 98, 99, or 100% homogenous with respect to other polynucleotide molecules. In many embodiments of the invention, a nucleic acid is isolated by virtue of it having been synthesized in vitro separate from endogenous nucleic acids in a cell. It will be understood, however, that isolated nucleic acids may be subsequently mixed or pooled together. In certain aspects, synthetic miRNA of the invention are RNA or RNA analogs. miRNA inhibitors may be DNA or RNA, or analogs thereof. miRNA and miRNA inhibitors of the invention are collectively referred to as “synthetic nucleic acids.”

In some embodiments, there is a miRNA or a synthetic miRNA having a length of between 17 and 130 residues. The present invention concerns miRNA or synthetic miRNA molecules that are, are at least, or are at most 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 140, 145, 150, 160, 170, 180, 190, 200 or more residues in length, including any integer or any range there between.

In certain embodiments, synthetic miRNA have (a) a “miRNA region” whose sequence or binding region from 5′ to 3′ is identical or complementary to all or a segment of a mature miRNA sequence, and (b) a “complementary region” whose sequence from 5′ to 3′ is between 60% and 100% complementary to the miRNA sequence in (a). In certain embodiments, these synthetic miRNA are also isolated, as defined above. The term “miRNA region” refers to a region on the synthetic miRNA that is at least 75, 80, 85, 90, 95, or 100% identical, including all integers there between, to the entire sequence of a mature, naturally occurring miRNA sequence or a complement thereof. In certain embodiments, the miRNA region is or is at least 90, 91, 92, 93, 94, 95, 96, 97, 98, 99, 99.1, 99.2, 99.3, 99.4, 99.5, 99.6, 99.7, 99.8, 99.9 or 100% identical to the sequence of a naturally-occurring miRNA or complement thereof.

The term “complementary region” or “complement” refers to a region of a nucleic acid or mimetic that is or is at least 60% complementary to the mature, naturally occurring miRNA sequence. The complementary region is or is at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 99.1, 99.2, 99.3, 99.4, 99.5, 99.6, 99.7, 99.8, 99.9 or 100% complementary, or any range derivable therein. With single polynucleotide sequences, there may be a hairpin loop structure as a result of chemical bonding between the miRNA region and the complementary region. In other embodiments, the complementary region is on a different nucleic acid molecule than the miRNA region, in which case the complementary region is on the complementary strand and the miRNA region is on the active strand.

In other embodiments of the invention, there are synthetic nucleic acids that are miRNA inhibitors. A miRNA inhibitor is between about 17 to 25 nucleotides in length and comprises a 5′ to 3′ sequence that is at least 90% complementary to the 5′ to 3′ sequence of a mature miRNA. In certain embodiments, a miRNA inhibitor molecule is 17, 18, 19, 20, 21, 22, 23, 24, or 25 nucleotides in length, or any range derivable therein. Moreover, an miRNA inhibitor may have a sequence (from 5′ to 3′) that is or is at least 70, 75, 80, 85, 90, 91, 92, 93, 94, 95, 96, 97, 98, 99, 99.1, 99.2, 99.3, 99.4, 99.5, 99.6, 99.7, 99.8, 99.9 or 100% complementary, or any range derivable therein, to the 5′ to 3′ sequence of a mature miRNA, particularly a mature, naturally occurring miRNA. One of skill in the art could use a portion of the miRNA sequence that is complementary to the sequence of a mature miRNA as the sequence for a miRNA inhibitor. Moreover, that portion of the nucleic acid sequence can be altered so that it is still comprises the appropriate percentage of complementarity to the sequence of a mature miRNA.

In some embodiments, of the invention, a synthetic miRNA or inhibitor contains one or more design element(s). These design elements include, but are not limited to: (i) a replacement group for the phosphate or hydroxyl of the nucleotide at the 5′ terminus of the complementary region; (ii) one or more sugar modifications in the first or last 1 to 6 residues of the complementary region; or, (iii) noncomplementarity between one or more nucleotides in the last 1 to 5 residues at the 3′ end of the complementary region and the corresponding nucleotides of the miRNA region. A variety of design modifications are known in the art, see below.

In certain embodiments, a synthetic miRNA has a nucleotide at its 5′ end of the complementary region in which the phosphate and/or hydroxyl group has been replaced with another chemical group (referred to as the “replacement design”). In some cases, the phosphate group is replaced, while in others, the hydroxyl group has been replaced. In particular embodiments, the replacement group is biotin, an amine group, a lower alkylamine group, an aminohexyl phosphate group, an acetyl group, 2′0-Me (2′ oxygen-methyl), DMTO (4,4′-dimethoxytrityl with oxygen), fluorescein, a thiol, or acridine, though other replacement groups are well known to those of skill in the art and can be used as well. This design element can also be used with a miRNA inhibitor.

Additional embodiments concern a synthetic miRNA having one or more sugar modifications in the first or last 1 to 6 residues of the complementary region (referred to as the 6“sugar replacement design”). In certain cases, there is one or more sugar modifications in the first 1, 2, 3, 4, 5, 6 or more residues of the complementary region, or any range derivable therein. In additional cases, there is one or more sugar modifications in the last 1, 2, 3, 4, 5, 6 or more residues of the complementary region, or any range derivable therein, have a sugar modification. It will be understood that the terms “first” and “last” are with respect to the order of residues from the 5′ end to the 3′ end of the region. In particular embodiments, the sugar modification is a 2′O-Me, a 2° F. modification, a 2′H modification, a 2′ amino modification, a 4′thioribose modification, or a phosphorothioate modification on the carboxy group linked to the carbon at position 6′. In further embodiments, there is one or more sugar modifications in the first or last 2 to 4 residues of the complementary region or the first or last 4 to 6 residues of the complementary region. This design element can also be used with an miRNA inhibitor. Thus, an miRNA inhibitor can have this design element and/or a replacement group on the nucleotide at the 5′ terminus, as discussed above.

In other embodiments of the invention, there is a synthetic miRNA or inhibitor in which one or more nucleotides in the last 1 to 5 residues at the 3′ end of the complementary region are not complementary to the corresponding nucleotides of the miRNA region (“noncomplementarity”) (referred to as the “noncomplementarity design”). The noncomplementarity may be in the last 1, 2, 3, 4, and/or 5 residues of the complementary miRNA. In certain embodiments, there is noncomplementarity with at least 2 nucleotides in the complementary region.

It is contemplated that synthetic miRNA of the invention have one or more of the replacement, sugar modification, or noncomplementarity designs. In certain cases, synthetic RNA molecules have two of them, while in others these molecules have all three designs in place.

The miRNA region and the complementary region may be on the same or separate polynucleotides. In cases in which they are contained on or in the same polynucleotide, the miRNA molecule will be considered a single polynucleotide. In embodiments in which the different regions are on separate polynucleotides, the synthetic miRNA will be considered to be comprised of two polynucleotides.

When the RNA molecule is a single polynucleotide, there can be a linker region between the miRNA region and the complementary region. In some embodiments, the single polynucleotide is capable of forming a hairpin loop structure as a result of bonding between the miRNA region and the complementary region. The linker constitutes the hairpin loop. It is contemplated that in some embodiments, the linker region is, is at least, or is at most 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 residues in length, or any range derivable therein. In certain embodiments, the linker is between 3 and 30 residues (inclusive) in length.

In addition to having a miRNA or inhibitor region and a complementary region, there may be flanking sequences as well at either the 5′ or 3′ end of the region. In some embodiments, there is or is at least 1, 2, 3, 4, 5, 6, 7, 8, 9, 10 nucleotides or more, or any range derivable therein, flanking one or both sides of these regions.

Methods of the invention include reducing or eliminating activity of one or more miRNAs in a cell comprising introducing into a cell a miRNA inhibitor (which may be described generally herein as an miRNA, so that a description of miRNA, where appropriate, also will refer to a miRNA inhibitor); or supplying or enhancing the activity of one or more miRNAs in a cell. The present invention also concerns inducing certain cellular characteristics by providing to a cell a particular nucleic acid, such as a specific synthetic miRNA molecule or a synthetic miRNA inhibitor molecule. However, in methods of the invention, the miRNA molecule or miRNA inhibitor need not be synthetic. They may have a sequence that is identical to a naturally occurring miRNA or they may not have any design modifications. In certain embodiments, the miRNA molecule and/or the miRNA inhibitor are synthetic, as discussed above.

The particular nucleic acid molecule provided to the cell is understood to correspond to a particular miRNA in the cell, and thus, the miRNA in the cell is referred to as the “corresponding miRNA.” In situations in which a named miRNA molecule is introduced into a cell, the corresponding miRNA will be understood to be the induced or inhibited miRNA or induced or inhibited miRNA function. It is contemplated, however, that the miRNA molecule introduced into a cell is not a mature miRNA but is capable of becoming or functioning as a mature miRNA under the appropriate physiological conditions. In cases in which a particular corresponding miRNA is being inhibited by a miRNA inhibitor, the particular miRNA will be referred to as the “targeted miRNA.” It is contemplated that multiple corresponding miRNAs may be involved. In particular embodiments, more than one miRNA molecule is introduced into a cell. Moreover, in other embodiments, more than one miRNA inhibitor is introduced into a cell. Furthermore, a combination of miRNA molecule(s) and miRNA inhibitor(s) may be introduced into a cell. The inventors contemplate that a combination of miRNA may act at one or more points in cellular pathways of cells with aberrant phenotypes and that such combination may have increased efficacy on the target cell while not adversely effecting normal cells. Thus, a combination of miRNA may have a minimal adverse effect on a subject or patient while supplying a sufficient therapeutic effect, such as amelioration of a condition, growth inhibition of a cell, death of a targeted cell, alteration of cell phenotype or physiology, slowing of cellular growth, sensitization to a second therapy, sensitization to a particular therapy, and the like.

Methods include identifying a cell or patient in need of inducing those cellular characteristics. Also, it will be understood that an amount of a synthetic nucleic acid that is provided to a cell or organism is an “effective amount,” which refers to an amount needed (or a sufficient amount) to achieve a desired goal, such as inducing a particular cellular characteristic(s). Certain embodiments of the methods include providing or introducing to a cell a nucleic acid molecule corresponding to a mature miRNA in the cell in an amount effective to achieve a desired physiological result.

Moreover, methods can involve providing synthetic or nonsynthetic miRNA molecules. It is contemplated that in these embodiments, that the methods may or may not be limited to providing only one or more synthetic miRNA molecules or only one or more nonsynthetic miRNA molecules. Thus, in certain embodiments, methods may involve providing both synthetic and nonsynthetic miRNA molecules. In this situation, a cell or cells are most likely provided a synthetic miRNA molecule corresponding to a particular miRNA and a nonsynthetic miRNA molecule corresponding to a different miRNA. Furthermore, any method articulated using a list of miRNAs using Markush group language may be articulated without the Markush group language and a disjunctive article (i.e., or) instead, and vice versa.

Typically, an endogenous gene, miRNA or mRNA is modulated in the cell. In particular embodiments, the nucleic acid sequence comprises at least one segment that is at least 70, 75, 80, 85, 90, 95, or 100% identical in nucleic acid sequence to one or more miRNA or gene sequence. Modulation of the expression or processing of an endogenous gene, miRNA, or mRNA can be through modulation of the processing of a mRNA, such processing including transcription, transportation and/or translation with in a cell. Modulation may also be effected by the inhibition or enhancement of miRNA activity with a cell, tissue, or organ. Such processing may affect the expression of an encoded product or the stability of the mRNA. In still other embodiments, a nucleic acid sequence can comprise a modified nucleic acid sequence. In certain aspects, one or more miRNA sequence may include or comprise a modified nucleobase or nucleic acid sequence.

It will be understood in methods of the invention that a cell or other biological matter such as an organism (including patients) can be provided a miRNA or miRNA molecule corresponding to a particular miRNA by administering to the cell or organism a nucleic acid molecule that functions as the corresponding miRNA once inside the cell. The form of the molecule provided to the cell may not be the form that acts a miRNA once inside the cell. Thus, it is contemplated that in some embodiments, a synthetic miRNA or a nonsynthetic miRNA is provided such that it becomes processed into a mature and active miRNA once it has access to the cell's miRNA processing machinery. In certain embodiments, it is specifically contemplated that the miRNA molecule provided is not a mature miRNA molecule but a nucleic acid molecule that can be processed into the mature miRNA once it is accessible to miRNA processing machinery. The term “nonsynthetic” in the context of miRNA means that the miRNA is not “synthetic,” as defined herein. Furthermore, it is contemplated that in embodiments of the invention that concern the use of synthetic miRNAs, the use of corresponding nonsynthetic miRNAs is also considered an aspect of the invention, and vice versa. It will be understand that the term “providing” an agent is used to include “administering” the agent to a patient.

In certain embodiments, methods also include targeting a miRNA to modulate in a cell or organism. The term “targeting a miRNA to modulate” means a nucleic acid of the invention will be employed so as to modulate the selected miRNA. In some embodiments the modulation is achieved with a synthetic or non-synthetic miRNA that corresponds to the targeted miRNA, which effectively provides the targeted miRNA to the cell or organism (positive modulation). In other embodiments, the modulation is achieved with a miRNA inhibitor, which effectively inhibits the targeted miRNA in the cell or organism (negative modulation).

In some embodiments, the miRNA targeted to be modulated is a miRNA that affects a disease, condition, or pathway. In certain embodiments, the miRNA is targeted because a treatment can be provided by negative modulation of the targeted miRNA. In other embodiments, the miRNA is targeted because a treatment can be provided by positive modulation of the targeted miRNA or its targets.

In certain methods of the invention, there is a further step of administering the selected miRNA modulator to a cell, tissue, organ, or organism (collectively “biological matter”) in need of treatment related to modulation of the targeted miRNA or in need of the physiological or biological results discussed herein (such as with respect to a particular cellular pathway or result like decrease in cell viability). Consequently, in some methods of the invention there is a step of identifying a patient in need of treatment that can be provided by the miRNA modulator(s). It is contemplated that an effective amount of a miRNA modulator can be administered in some embodiments. In particular embodiments, there is a therapeutic benefit conferred on the biological matter, where a “therapeutic benefit” refers to an improvement in the one or more conditions or symptoms associated with a disease or condition or an improvement in the prognosis, duration, or status with respect to the disease. It is contemplated that a therapeutic benefit includes, but is not limited to, a decrease in pain, a decrease in morbidity, a decrease in a symptom. For example, with respect to cancer, it is contemplated that a therapeutic benefit can be inhibition of tumor growth, prevention of metastasis, reduction in number of metastases, inhibition of cancer cell proliferation, induction of cell death in cancer cells, inhibition of angiogenesis near cancer cells, induction of apoptosis of cancer cells, reduction in pain, reduction in risk of recurrence, induction of chemo- or radiosensitivity in cancer cells, prolongation of life, and/or delay of death directly or indirectly related to cancer.

Furthermore, it is contemplated that the miRNA compositions may be provided as part of a therapy to a patient, in conjunction with traditional therapies or preventative agents. Moreover, it is contemplated that any method discussed in the context of therapy may be applied preventatively, particularly in a patient identified to be potentially in need of the therapy or at risk of the condition or disease for which a therapy is needed.

In addition, methods of the invention concern employing one or more nucleic acids corresponding to a miRNA and a therapeutic drug. The nucleic acid can enhance the effect or efficacy of the drug, reduce any side effects or toxicity, modify its bioavailability, and/or decrease the dosage or frequency needed. In certain embodiments, the therapeutic drug is a cancer therapeutic. Consequently, in some embodiments, there is a method of treating cancer in a patient comprising administering to the patient the cancer therapeutic and an effective amount of at least one miRNA molecule that improves the efficacy of the cancer therapeutic or protects non-cancer cells. Cancer therapies also include a variety of combination therapies with both chemical and radiation based treatments. Combination chemotherapies include but are not limited to, for example, 5-fluorouracil, alemtuzumab, amrubicin, bevacizumab, bleomycin, bortezomib, busulfan, camptothecin, capecitabine, carboplatin, cetuximab, chlorambucil, cisplatin (CDDP), COX-2 inhibitors (e.g., celecoxib), cyclophosphamide, cytarabine, dactinomycin, dasatinib, daunorubicin, dexamethasone, docetaxel, doxorubicin (adriamycin), EGFR inhibitors (gefitinib and cetuximab), erlotinib, estrogen receptor binding agents, etoposide (VP16), everolimus, farnesyl-protein transferase inhibitors, gefitinib, gemcitabine, gemtuzumab, ibritumomab, ifosfamide, imatinib mesylate, larotaxel, lapatinib, lonafarnib, mechlorethamine, melphalan, methotrexate, mitomycin, navelbine, nitrosurea, nocodazole, oxaliplatin, paclitaxel, plicomycin, procarbazine, raloxifene, rituximab, sirolimus, sorafenib, sunitinib, tamoxifen, taxol, taxotere, temsirolimus, tipifarnib, tositumomab, transplatinum, trastuzumab, vinblastin, vincristin, or vinorelbine or any analog or derivative variant of the foregoing.

Generally, inhibitors of miRNAs can be given to decrease the activity of an endogenous miRNA. For example, inhibitors of miRNA molecules that increase cell proliferation can be provided to cells to decrease cell proliferation. The present invention contemplates these embodiments in the context of the different physiological effects observed with the different miRNA molecules and miRNA inhibitors disclosed herein. These include, but are not limited to, the following physiological effects: increase and decreasing cell proliferation, increasing or decreasing apoptosis, increasing transformation, increasing or decreasing cell viability, activating or inhibiting a kinase (e.g., Erk), activating/inducing or inhibiting hTert, inhibit stimulation of growth promoting pathway (e.g., Stat 3 signaling), reduce or increase viable cell number, and increase or decrease number of cells at a particular phase of the cell cycle. Methods of the invention are generally contemplated to include providing or introducing one or more different nucleic acid molecules corresponding to one or more different miRNA molecules. It is contemplated that the following, at least the following, or at most the following number of different nucleic acid or miRNA molecules may be provided or introduced: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or any range derivable therein. This also applies to the number of different miRNA molecules that can be provided or introduced into a cell.

### II. Pharmaceutical Formulations and Delivery

Methods of the present invention include the delivery of an effective amount of a miRNA or an expression construct encoding the same. An “effective amount” of the pharmaceutical composition, generally, is defined as that amount sufficient to detectably and repeatedly to achieve the stated desired result, for example, to ameliorate, reduce, minimize or limit the extent of the disease or its symptoms. Other more rigorous definitions may apply, including elimination, eradication or cure of disease.

A. Administration

In certain embodiments, it is desired to kill cells, inhibit cell growth, inhibit metastasis, decrease tumor or tissue size, and/or reverse or reduce the malignant or disease phenotype of cells. The routes of administration will vary, naturally, with the location and nature of the lesion or site to be targeted, and include, e.g., intradermal, subcutaneous, regional, parenteral, intravenous, intramuscular, intranasal, systemic, and oral administration and formulation. Direct injection, intratumoral injection, or injection into tumor vasculature is specifically contemplated for discrete, solid, accessible tumors, or other accessible target areas. Local, regional, or systemic administration also may be appropriate. For tumors of >4 cm, the volume to be administered will be about 4-10 ml (preferably 10 ml), while for tumors of <4 cm, a volume of about 1-3 ml will be used (preferably 3 ml).

Multiple injections delivered as a single dose comprise about 0.1 to about 0.5 ml volumes. Compositions of the invention may be administered in multiple injections to a tumor or a targeted site. In certain aspects, injections may be spaced at approximately 1 cm intervals.

In the case of surgical intervention, the present invention may be used preoperatively, to render an inoperable tumor subject to resection. Alternatively, the present invention may be used at the time of surgery, and/or thereafter, to treat residual or metastatic disease. For example, a resected tumor bed may be injected or perfused with a formulation comprising a miRNA or combinations thereof. Administration may be continued post-resection, for example, by leaving a catheter implanted at the site of the surgery. Periodic post-surgical treatment also is envisioned. Continuous perfusion of an expression construct or a viral construct also is contemplated.

Continuous administration also may be applied where appropriate, for example, where a tumor or other undesired affected area is excised and the tumor bed or targeted site is treated to eliminate residual, microscopic disease. Delivery via syringe or catherization is contemplated. Such continuous perfusion may take place for a period from about 1-2 hours, to about 2-6 hours, to about 6-12 hours, to about 12-24 hours, to about 1-2 days, to about 1-2 wk or longer following the initiation of treatment. Generally, the dose of the therapeutic composition via continuous perfusion will be equivalent to that given by a single or multiple injections, adjusted over a period of time during which the perfusion occurs.

Treatment regimens may vary as well and often depend on tumor type, tumor location, immune condition, target site, disease progression, and health and age of the patient. Certain tumor types will require more aggressive treatment. The clinician will be best suited to make such decisions based on the known efficacy and toxicity (if any) of the therapeutic formulations.

In certain embodiments, the tumor or affected area being treated may not, at least initially, be resectable. Treatments with compositions of the invention may increase the resectability of the tumor due to shrinkage at the margins or by elimination of certain particularly invasive portions. Following treatments, resection may be possible. Additional treatments subsequent to resection may serve to eliminate microscopic residual disease at the tumor or targeted site.

Treatments may include various “unit doses.” A unit dose is defined as containing a predetermined quantity of a therapeutic composition(s). The quantity to be administered, and the particular route and formulation, are within the skill of those in the clinical arts. A unit dose need not be administered as a single injection but may comprise continuous infusion over a set period of time. With respect to a viral component of the present invention, a unit dose may conveniently be described in terms of μg or mg of miRNA or miRNA mimetic. Alternatively, the amount specified may be the amount administered as the average daily, average weekly, or average monthly dose.

miRNA can be administered to the patient in a dose or doses of about or of at least about 0.5, 1, 5, 10, 15, 20, 25, 30, 35, 40, 45, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000 μg or mg, or more, or any range derivable therein. Alternatively, the amount specified may be the amount administered as the average daily, average weekly, or average monthly dose, or it may be expressed in terms of mg/kg, where kg refers to the weight of the patient and the mg is specified above. In other embodiments, the amount specified is any number discussed above but expressed as mg/m² (with respect to tumor size or patient surface area).

B. Injectable Compositions and Formulations

In some embodiments, the method for the delivery of a miRNA or an expression construct encoding such or combinations thereof is via systemic administration. However, the pharmaceutical compositions disclosed herein may also be administered parenterally, subcutaneously, directly, intratracheally, intravenously, intradermally, intramuscularly, or even intraperitoneally as described in U.S. Pat. Nos. 5,543,158; 5,641,515 and 5,399,363 (each specifically incorporated herein by reference in its entirety).

Injection of nucleic acids may be delivered by syringe or any other method used for injection of a solution, as long as the nucleic acid and any associated components can pass through the particular gauge of needle required for injection. A syringe system has also been described for use in gene therapy that permits multiple injections of predetermined quantities of a solution precisely at any depth (U.S. Pat. No. 5,846,225).

Solutions of the active compounds as free base or pharmacologically acceptable salts may be prepared in water suitably mixed with a surfactant, such as hydroxypropylcellulose. Dispersions may also be prepared in glycerol, liquid polyethylene glycols, mixtures thereof, and in oils. Under ordinary conditions of storage and use, these preparations contain a preservative to prevent the growth of microorganisms. The pharmaceutical forms suitable for injectable use include sterile aqueous solutions or dispersions and sterile powders for the extemporaneous preparation of sterile injectable solutions or dispersions (U.S. Pat. No. 5,466,468, specifically incorporated herein by reference in its entirety). In all cases the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

In certain formulations, a water-based formulation is employed while in others, it may be lipid-based. In particular embodiments of the invention, a composition comprising a tumor suppressor protein or a nucleic acid encoding the same is in a water-based formulation. In other embodiments, the formulation is lipid based.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous, intratumoral, intralesional, and intraperitoneal administration. In this connection, sterile aqueous media which can be employed will be known to those of skill in the art in light of the present disclosure. For example, one dosage may be dissolved in 1 ml of isotonic NaCl solution and either added to 1000 ml of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics standards.

As used herein, a “carrier” includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions.

The phrase “pharmaceutically acceptable” refers to molecular entities and compositions that do not produce an allergic or similar untoward reaction when administered to a human.

The nucleic acid(s) are administered in a manner compatible with the dosage formulation, and in such amount as will be therapeutically effective. The quantity to be administered depends on the subject to be treated, including, e.g., the aggressiveness of the disease or cancer, the size of any tumor(s) or lesions, the previous or other courses of treatment. Precise amounts of active ingredient required to be administered depend on the judgment of the practitioner. Suitable regimes for initial administration and subsequent administration are also variable, but are typified by an initial administration followed by other administrations. Such administration may be systemic, as a single dose, continuous over a period of time spanning 10, 20, 30, 40, 50, 60 minutes, and/or 1, 2, 3, 4, 5, 6, 7, 8, 9, 10, 11, 12, 13, 14, 15, 16, 17, 18, 19, 20, 21, 22, 23, 24 or more hours, and/or 1, 2, 3, 4, 5, 6, 7, days or more. Moreover, administration may be through a time release or sustained release mechanism, implemented by formulation and/or mode of administration.

Various methods for nucleic acid delivery are described, for example in Sambrook et al., 1989 and Ausubel et al., 1994. Such nucleic acid delivery systems comprise the desired nucleic acid, by way of example and not by limitation, in either “naked” form as a “naked” nucleic acid, or formulated in a vehicle suitable for delivery, such as in a complex with a cationic molecule or a liposome forming lipid, or as a component of a vector, or a component of a pharmaceutical composition. The nucleic acid delivery system can be provided to the cell either directly, such as by contacting it with the cell, or indirectly, such as through the action of any biological process. By way of example, and not by limitation, the nucleic acid delivery system can be provided to the cell by endocytosis; receptor targeting; coupling with native or synthetic cell membrane fragments; physical means such as electroporation; combining the nucleic acid delivery system with a polymeric carrier, such as a controlled release film or nanoparticle or microparticle or biocompatible molecules or biodegradable molecules; with vector. The nucleic acid delivery system can be injected into a tissue or fluid surrounding the cell, or administered by diffusion of the nucleic acid delivery system across the cell membrane, or by any active or passive transport mechanism across the cell membrane. Additionally, the nucleic acid delivery system can be provided to the cell using techniques such as antibody-related targeting and antibody-mediated immobilization of a viral vector.

C. Combination Treatments

In certain embodiments, the compositions and methods of the present invention involve a miRNA, or expression construct encoding such. These miRNA composition can be used in combination with a second therapy to enhance the effect of the miRNA therapy, or increase the therapeutic effect of another therapy being employed. These compositions would be provided in a combined amount effective to achieve the desired effect, such as the killing of a cancer cell and/or the inhibition of cellular hyperproliferation. This process may involve contacting the cells with the miRNA or second therapy at the same or different time. This may be achieved by contacting the cell with one or more compositions or pharmacological formulation that includes or more of the agents, or by contacting the cell with two or more distinct compositions or formulations, wherein one composition provides (1) miRNA; and/or (2) a second therapy. A second composition or method may be administered that includes a chemotherapy, radiotherapy, surgical therapy, immunotherapy or gene therapy.

It is contemplated that one may provide a patient with the miRNA therapy and the second therapy within about 12-24 h of each other and, more preferably, within about 6-12 h of each other. In some situations, it may be desirable to extend the time period for treatment significantly, however, where several days (2, 3, 4, 5, 6 or 7) to several weeks (1, 2, 3, 4, 5, 6, 7 or 8) lapse between the respective administrations.

In certain embodiments, a course of treatment will last 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90 days or more. It is contemplated that one agent may be given on day 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, and/or 90, any combination thereof, and another agent is given on day 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, and/or 90, or any combination thereof. Within a single day (24-hour period), the patient may be given one or multiple administrations of the agent(s). Moreover, after a course of treatment, it is contemplated that there is a period of time at which no treatment is administered. This time period may last 1, 2, 3, 4, 5, 6, 7 days, and/or 1, 2, 3, 4, 5 weeks, and/or 1, 2, 3, 4, 5, 6, 7, 8, 9, 10, 11, 12 months or more, depending on the condition of the patient, such as their prognosis, strength, health, etc.

Various combinations may be employed, for example miRNA therapy is “A” and a second therapy is “B”:

A/B/A B/A/B B/B/A A/A/B A/B/B B/A/A A/B/B/B
B/A/B/B B/B/B/A B/B/A/B A/A/B/B A/B/A/B A/B/B/A
B/B/A/A B/A/B/A B/A/A/B A/A/A/B B/A/A/A A/B/A/A
A/A/B/A

Administration of any compound or therapy of the present invention to a patient will follow general protocols for the administration of such compounds, taking into account the toxicity, if any, of the vector or any protein or other agent. Therefore, in some embodiments there is a step of monitoring toxicity that is attributable to combination therapy. It is expected that the treatment cycles would be repeated as necessary. It also is contemplated that various standard therapies, as well as surgical intervention, may be applied in combination with the described therapy.

In specific aspects, it is contemplated that a second therapy, such as chemotherapy, radiotherapy, immunotherapy, surgical therapy or other gene therapy, is employed in combination with the miRNA therapy, as described herein.

1. Chemotherapy

A wide variety of chemotherapeutic agents may be used in accordance with the present invention. The term “chemotherapy” refers to the use of drugs to treat cancer. A “chemotherapeutic agent” is used to connote a compound or composition that is administered in the treatment of cancer. These agents or drugs are categorized by their mode of activity within a cell, for example, whether and at what stage they affect the cell cycle. Alternatively, an agent may be characterized based on its ability to directly cross-link DNA, to intercalate into DNA, or to induce chromosomal and mitotic aberrations by affecting nucleic acid synthesis. Most chemotherapeutic agents fall into the following categories: alkylating agents, antimetabolites, antitumor antibiotics, mitotic inhibitors, and nitrosoureas.

a. Alkylating Agents

Alkylating agents are drugs that directly interact with genomic DNA to prevent the cancer cell from proliferating. This category of chemotherapeutic drugs represents agents that affect all phases of the cell cycle, that is, they are not phase-specific. Alkylating agents can be implemented to treat chronic leukemia, non-Hodgkin's lymphoma, Hodgkin's disease, multiple myeloma, and particular cancers of the breast, lung, and ovary. They include: busulfan, chlorambucil, cisplatin, cyclophosphamide (cytoxan), dacarbazine, ifosfamide, mechlorethamine (mustargen), and melphalan. Troglitazaone can be used to treat cancer in combination with any one or more of these alkylating agents.

b. Antimetabolites

Antimetabolites disrupt DNA and RNA synthesis. Unlike alkylating agents, they specifically influence the cell cycle during S phase. They have been used to combat chronic leukemias in addition to tumors of breast, ovary and the gastrointestinal tract. Antimetabolites include 5-fluorouracil (5-FU), cytarabine (Ara-C), fludarabine, gemcitabine, and methotrexate.

5-Fluorouracil (5-FU) has the chemical name of 5-fluoro-2,4(1H,3H)-pyrimidinedione. Its mechanism of action is thought to be by blocking the methylation reaction of deoxyuridylic acid to thymidylic acid. Thus, 5-FU interferes with the synthesis of deoxyribonucleic acid (DNA) and to a lesser extent inhibits the formation of ribonucleic acid (RNA). Since DNA and RNA are essential for cell division and proliferation, it is thought that the effect of 5-FU is to create a thymidine deficiency leading to cell death. Thus, the effect of 5-FU is found in cells that rapidly divide, a characteristic of metastatic cancers.

c. Antitumor Antibiotics

Antitumor antibiotics have both antimicrobial and cytotoxic activity. These drugs also interfere with DNA by chemically inhibiting enzymes and mitosis or altering cellular membranes. These agents are not phase specific so they work in all phases of the cell cycle. Thus, they are widely used for a variety of cancers. Examples of antitumor antibiotics include bleomycin, dactinomycin, daunorubicin, doxorubicin (Adriamycin), and idarubicin, some of which are discussed in more detail below. Widely used in clinical setting for the treatment of neoplasms, these compounds are administered through bolus injections intravenously at doses ranging from 25-75 mg/m² at 21 day intervals for adriamycin, to 35-100 mg/m² for etoposide intravenously or orally.

d. Mitotic Inhibitors

Mitotic inhibitors include plant alkaloids and other natural agents that can inhibit either protein synthesis required for cell division or mitosis. They operate during a specific phase during the cell cycle. Mitotic inhibitors comprise docetaxel, etoposide (VP16), paclitaxel, taxol, taxotere, vinblastine, vincristine, and vinorelbine.

e. Nitrosureas

Nitrosureas, like alkylating agents, inhibit DNA repair proteins. They are used to treat non-Hodgkin's lymphomas, multiple myeloma, malignant melanoma, in addition to brain tumors. Examples include carmustine and lomustine.

2. Radiotherapy

Radiotherapy, also called radiation therapy, is the treatment of cancer and other diseases with ionizing radiation. Ionizing radiation deposits energy that injures or destroys cells in the area being treated by damaging their genetic material, making it impossible for these cells to continue to grow. Although radiation damages both cancer cells and normal cells, the latter are able to repair themselves and function properly. Radiotherapy may be used to treat localized solid tumors, such as cancers of the skin, tongue, larynx, brain, breast, or cervix. It can also be used to treat leukemia and lymphoma (cancers of the blood-forming cells and lymphatic system, respectively).

Radiation therapy used according to the present invention may include, but is not limited to, the use of γ-rays, X-rays, and/or the directed delivery of radioisotopes to tumor cells. Other forms of DNA damaging factors are also contemplated such as microwaves, proton beam irradiation (U.S. Pat. Nos. 5,760,395 and 4,870,287) and UV-irradiation. It is most likely that all of these factors effect a broad range of damage on DNA, on the precursors of DNA, on the replication and repair of DNA, and on the assembly and maintenance of chromosomes. Dosage ranges for X-rays range from daily doses of 50 to 200 roentgens for prolonged periods of time (3 to 4 wk), to single doses of 2000 to 6000 roentgens. Dosage ranges for radioisotopes vary widely, and depend on the half-life of the isotope, the strength and type of radiation emitted, and the uptake by the neoplastic cells. Radiotherapy may comprise the use of radiolabeled antibodies to deliver doses of radiation directly to the cancer site (radioimmunotherapy). Once injected into the body, the antibodies actively seek out the cancer cells, which are destroyed by the cell-killing (cytotoxic) action of the radiation. This approach can minimize the risk of radiation damage to healthy cells.

Stereotactic radio-surgery (gamma knife) for brain and other tumors does not use a knife, but very precisely targeted beams of gamma radiotherapy from hundreds of different angles. Only one session of radiotherapy, taking about four to five hours, is needed. For this treatment a specially made metal frame is attached to the head. Then, several scans and x-rays are carried out to find the precise area where the treatment is needed. During the radiotherapy for brain tumors, the patient lies with their head in a large helmet, which has hundreds of holes in it to allow the radiotherapy beams through. Related approaches permit positioning for the treatment of tumors in other areas of the body.

3. Immunotherapy

In the context of cancer treatment, immunotherapeutics, generally, rely on the use of immune effector cells and molecules to target and destroy cancer cells. Trastuzumab (Herceptin™) is such an example. The immune effector may be, for example, an antibody specific for some marker on the surface of a tumor cell. The antibody alone may serve as an effector of therapy or it may recruit other cells to actually affect cell killing. The antibody also may be conjugated to a drug or toxin (chemotherapeutic, radionuclide, ricin A chain, cholera toxin, pertussis toxin, etc.) and serve merely as a targeting agent. Alternatively, the effector may be a lymphocyte carrying a surface molecule that interacts, either directly or indirectly, with a tumor cell target. Various effector cells include cytotoxic T cells and NK cells. The combination of therapeutic modalities, i.e., direct cytotoxic activity and inhibition or reduction of ErbB2 would provide therapeutic benefit in the treatment of ErbB2 overexpressing cancers.

In one aspect of immunotherapy, the tumor or disease cell must bear some marker that is amenable to targeting, i.e., is not present on the majority of other cells. Many tumor markers exist and any of these may be suitable for targeting in the context of the present invention. Common tumor markers include carcinoembryonic antigen, prostate specific antigen, urinary tumor associated antigen, fetal antigen, tyrosinase (p97), gp68, TAG-72, HMFG, Sialyl Lewis Antigen, MucA, MucB, PLAP, estrogen receptor, laminin receptor, erb B and p155. An alternative aspect of immunotherapy is to combine anticancer effects with immune stimulatory effects. Immune stimulating molecules also exist including: cytokines such as IL-2, IL-4, IL-12, GM-CSF, gamma-IFN, chemokines such as MIP-1, MCP-1, IL-8 and growth factors such as FLT3 ligand. Combining immune stimulating molecules, either as proteins or using gene delivery in combination with a tumor suppressor such as MDA-7 has been shown to enhance anti-tumor effects (Ju et al., 2000). Moreover, antibodies against any of these compounds can be used to target the anti-cancer agents discussed herein.

Examples of immunotherapies currently under investigation or in use are immune adjuvants e.g., Mycobacterium bovis, Plasmodium falciparum, dinitrochlorobenzene and aromatic compounds (U.S. Pat. Nos. 5,801,005 and 5,739,169; Hui and Hashimoto, 1998; Christodoulides et al., 1998), cytokine therapy e.g., interferons α, β and γ; IL-1, GM-CSF and TNF (Bukowski et al., 1998; Davidson et al., 1998; Hellstrand et al., 1998) gene therapy e.g., TNF, IL-1, IL-2, p53 (Qin et al., 1998; Austin-Ward and Villaseca, 1998; U.S. Pat. Nos. 5,830,880 and 5,846,945) and monoclonal antibodies e.g., anti-ganglioside GM2, anti-HER-2, anti-p185; Pietras et al., 1998; Hanibuchi et al., 1998; U.S. Pat. No. 5,824,311). Herceptin (trastuzumab) is a chimeric (mouse-human) monoclonal antibody that blocks the HER2-neu receptor. It possesses anti-tumor activity and has been approved for use in the treatment of malignant tumors (Dillman, 1999). Table 6 is a non-limiting list of several known anti-cancer immunotherapeutic agents and their targets. It is contemplated that one or more of these therapies may be employed with the miRNA therapies described herein.

A number of different approaches for passive immunotherapy of cancer exist. They may be broadly categorized into the following: injection of antibodies alone; injection of antibodies couple to toxins or chemotherapeutic agents; injection of antibodies coupled to radioactive isotopes; injection of anti-idiotype antibodies; and finally, purging of tumor cells in bone marrow.

TABLE 6

| Generic Name | Target |
|---|---|
| Cetuximab | EGFR |
| Panitumumab | EGFR |
| Trastuzumab | erbB2 receptor |
| Bevacizumab | VEGF |
| Alemtuzumab | CD52 |
| Gemtuzumab ozogamicin | CD33 |
| Rituximab | CD20 |
| Tositumomab | CD20 |
| Matuzumab | EGFR |
| Ibritumomab tiuxetan | CD20 |
| Tositumomab | CD20 |
| HuPAM4 | MUC1 |
| MORAb-009 | Mesothelin |
| G250 | carbonic anhydrase IX |
| mAb 8H9 | 8H9 antigen |
| M195 | CD33 |
| Ipilimumab | CTLA4 |
| HuLuc63 | CS1 |
| Alemtuzumab | CD53 |
| Epratuzumab | CD22 |
| BC8 | CD45 |
| HuJ591 | Prostate specific membrane antigen |
| hA20 | CD20 |
| Lexatumumab | TRAIL receptor-2 |
| Pertuzumab | HER-2 receptor |
| Mik-beta-1 | IL-2R |
| RAV12 | RAAG12 |
| SGN-30 | CD30 |
| AME-133v | CD20 |
| HeFi-1 | CD30 |
| BMS-663513 | CD137 |
| Volociximab | anti-α5β1 integrin |
| GC1008 | TGFβ |
| HCD122 | CD40 |
| Siplizumab | CD2 |
| MORAb-003 | Folate receptor alpha |
| CNTO 328 | IL-6 |
| MDX-060 | CD30 |
| Ofatumumab | CD20 |
| SGN-33 | CD33 |

4. Gene Therapy

In yet another embodiment, a combination treatment involves gene therapy in which a therapeutic polynucleotide is administered before, after, or at the same time as one or more therapeutic miRNA. Delivery of a therapeutic polypeptide or encoding nucleic acid in conjunction with a miRNA may have a combined therapeutic effect on target tissues. A variety of proteins are encompassed within the invention, some of which are described below. Various genes that may be targeted for gene therapy of some form in combination with the present invention include, but are not limited to inducers of cellular proliferation, inhibitors of cellular proliferation, regulators of programmed cell death, cytokines and other therapeutic nucleic acids or nucleic acid that encode therapeutic proteins.

The tumor suppressor oncogenes function to inhibit excessive cellular proliferation. The inactivation of these genes destroys their inhibitory activity, resulting in unregulated proliferation. The tumor suppressors (e.g., therapeutic polypeptides) p53, FHIT, p16 and C-CAM can be employed.

In addition to p53, another inhibitor of cellular proliferation is p16. The major transitions of the eukaryotic cell cycle are triggered by cyclin-dependent kinases, or CDK's. One CDK, cyclin-dependent kinase 4 (CDK4), regulates progression through the G1. The activity of this enzyme may be to phosphorylate Rb at late G1. The activity of CDK4 is controlled by an activating subunit, D-type cyclin, and by an inhibitory subunit, the p161NK4 has been biochemically characterized as a protein that specifically binds to and inhibits CDK4, and thus may regulate Rb phosphorylation (Serrano et al., 1993; Serrano et al., 1995). Since the p161NK4 protein is a CDK4 inhibitor (Serrano, 1993), deletion of this gene may increase the activity of CDK4, resulting in hyperphosphorylation of the Rb protein. p16 also is known to regulate the function of CDK6.

p161NK4 belongs to a newly described class of CDK-inhibitory proteins that also includes p16B, p19, p21WAF1, and p27KIP1. The p16INK4 gene maps to 9p21, a chromosome region frequently deleted in many tumor types. Homozygous deletions and mutations of the p161NK4 gene are frequent in human tumor cell lines. This evidence suggests that the p161NK4 gene is a tumor suppressor gene. This interpretation has been challenged, however, by the observation that the frequency of the p161NK4 gene alterations is much lower in primary uncultured tumors than in cultured cell lines (Caldas et al., 1994; Cheng et al., 1994; Hussussian et al., 1994; Kamb et al., 1994; Mori et al., 1994; Okamoto et al., 1994; Nobori et al., 1995; Orlow et al., 1994; Arap et al., 1995). Restoration of wild-type p161NK4 function by transfection with a plasmid expression vector reduced colony formation by some human cancer cell lines (Okamoto, 1994; Arap, 1995).

Other genes that may be employed according to the present invention include Rb, APC, DCC, NF-1, NF-2, WT-1, MEN-I, MEN-II, zac1, p73, VHL, MMAC1/PTEN, DBCCR-1, FCC, rsk-3, p27, p27/p16 fusions, p21/p27 fusions, anti-thrombotic genes (e.g., COX-1, TFPI), PGS, Dp, E2F, ras, myc, neu, raf, erb, fms, trk, ret, gsp, hst, abl, E1A, p300, genes involved in angiogenesis (e.g., VEGF, FGF, thrombospondin, BAI-1, GDAIF, or their receptors) and MCC.

5. Surgery

Approximately 60% of persons with cancer will undergo surgery of some type, which includes preventative, diagnostic or staging, curative and palliative surgery. Curative surgery is a cancer treatment that may be used in conjunction with other therapies, such as the treatment of the present invention, chemotherapy, radiotherapy, hormonal therapy, gene therapy, immunotherapy and/or alternative therapies.

Curative surgery includes resection in which all or part of cancerous tissue is physically removed, excised, and/or destroyed. Tumor resection refers to physical removal of at least part of a tumor. In addition to tumor resection, treatment by surgery includes laser surgery, cryosurgery, electrosurgery, and microscopically controlled surgery (Mohs' surgery). It is further contemplated that the present invention may be used in conjunction with removal of superficial cancers, precancers, or incidental amounts of normal tissue.

Upon excision of part of all of cancerous cells, tissue, or tumor, a cavity may be formed in the body. Treatment may be accomplished by perfusion, direct injection or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well.

6. Other Agents

It is contemplated that other agents may be used in combination with the present invention to improve the therapeutic efficacy of treatment. These additional agents include immunomodulatory agents, agents that affect the upregulation of cell surface receptors and GAP junctions, cytostatic and differentiation agents, inhibitors of cell adhesion, agents that increase the sensitivity of the hyperproliferative cells to apoptotic inducers, or other biological agents. Immunomodulatory agents include tumor necrosis factor; interferon alpha, beta, and gamma; IL-2 and other cytokines; F42K and other cytokine analogs; or MIP-1, MIP-1beta, MCP-1, RANTES, and other chemokines. It is further contemplated that the upregulation of cell surface receptors or their ligands such as Fas/Fas ligand, DR4 or DR5/TRAIL (Apo-2 ligand) would potentiate the apoptotic inducing abilities of the present invention by establishment of an autocrine or paracrine effect on hyperproliferative cells. Increases intercellular signaling by elevating the number of GAP junctions would increase the anti-hyperproliferative effects on the neighboring hyperproliferative cell population. In other embodiments, cytostatic or differentiation agents can be used in combination with the present invention to improve the anti-hyperproliferative efficacy of the treatments. Inhibitors of cell adhesion are contemplated to improve the efficacy of the present invention. Examples of cell adhesion inhibitors are focal adhesion kinase (FAKs) inhibitors and Lovastatin. It is further contemplated that other agents that increase the sensitivity of a hyperproliferative cell to apoptosis, such as the antibody c225, could be used in combination with the present invention to improve the treatment efficacy.

Apo2 ligand (Apo2L, also called TRAIL) is a member of the tumor necrosis factor (TNF) cytokine family. TRAIL activates rapid apoptosis in many types of cancer cells, yet is not toxic to normal cells. TRAIL mRNA occurs in a wide variety of tissues. Most normal cells appear to be resistant to TRAIL's cytotoxic action, suggesting the existence of mechanisms that can protect against apoptosis induction by TRAIL. The first receptor described for TRAIL, called death receptor 4 (DR4), contains a cytoplasmic “death domain”; DR4 transmits the apoptosis signal carried by TRAIL. Additional receptors have been identified that bind to TRAIL. One receptor, called DR5, contains a cytoplasmic death domain and signals apoptosis much like DR4. The DR4 and DR5 mRNAs are expressed in many normal tissues and tumor cell lines. Recently, decoy receptors such as DcR1 and DcR2 have been identified that prevent TRAIL from inducing apoptosis through DR4 and DR5. These decoy receptors thus represent a novel mechanism for regulating sensitivity to a pro-apoptotic cytokine directly at the cell's surface. The preferential expression of these inhibitory receptors in normal tissues suggests that TRAIL may be useful as an anticancer agent that induces apoptosis in cancer cells while sparing normal cells. (Marsters et al., 1999).

There have been many advances in the therapy of cancer following the introduction of cytotoxic chemotherapeutic drugs. However, one of the consequences of chemotherapy is the development/acquisition of drug-resistant phenotypes and the development of multiple drug resistance. The development of drug resistance remains a major obstacle in the treatment of such tumors and therefore, there is an obvious need for alternative approaches such as gene therapy.

Another form of therapy for use in conjunction with chemotherapy, radiation therapy or biological therapy includes hyperthermia, which is a procedure in which a patient's tissue is exposed to high temperatures (up to 106° F.). External or internal heating devices may be involved in the application of local, regional, or whole-body hyperthermia. Local hyperthermia involves the application of heat to a small area, such as a tumor. Heat may be generated externally with high-frequency waves targeting a tumor from a device outside the body. Internal heat may involve a sterile probe, including thin, heated wires or hollow tubes filled with warm water, implanted microwave antennae, or radiofrequency electrodes.

A patient's organ or a limb is heated for regional therapy, which is accomplished using devices that produce high energy, such as magnets. Alternatively, some of the patient's blood may be removed and heated before being perfused into an area that will be internally heated. Whole-body heating may also be implemented in cases where cancer has spread throughout the body. Warm-water blankets, hot wax, inductive coils, and thermal chambers may be used for this purpose.

Hormonal therapy may also be used in conjunction with the present invention or in combination with any other cancer therapy previously described. The use of hormones may be employed in the treatment of certain cancers such as breast, prostate, ovarian, or cervical cancer to lower the level or block the effects of certain hormones such as testosterone or estrogen. This treatment is often used in combination with at least one other cancer therapy as a treatment option or to reduce the risk of metastases.

This application incorporates U.S. application Ser. No. 11/349,727 filed on Feb. 8, 2006 claiming priority to U.S. Provisional Application Ser. No. 60/650,807 filed Feb. 8, 2005 herein by references in its entirety.

### III. Mirna Molecules

MicroRNA molecules (“miRNAs”) are generally 21 to 22 nucleotides in length, though lengths of 19 and up to 23 nucleotides have been reported. The miRNAs are each processed from a longer precursor RNA molecule (“precursor miRNA”). Precursor miRNAs are transcribed from non-protein-encoding genes. The precursor miRNAs have two regions of complementarity that enables them to form a stem-loop- or fold-back-like structure, which is cleaved in animals by a ribonuclease III-like nuclease enzyme called Dicer. The processed miRNA is typically a portion of the stem.

The processed miRNA (also referred to as “mature miRNA”) becomes part of a large complex to down-regulate a particular target gene or its gene product. Examples of animal miRNAs include those that imperfectly basepair with the target, which halts translation (Olsen et al., 1999; Seggerson et al., 2002). siRNA molecules also are processed by Dicer, but from a long, double-stranded RNA molecule. siRNAs are not naturally found in animal cells, but they can direct the sequence-specific cleavage of an mRNA target through a RNA-induced silencing complex (RISC) (Denli et al., 2003).

A. Array Preparation

Certain embodiments of the present invention concerns the preparation and use of mRNA or nucleic acid arrays, miRNA or nucleic acid arrays, and/or miRNA or nucleic acid probe arrays, which are macroarrays or microarrays of nucleic acid molecules (probes) that are fully or nearly complementary (over the length of the prove) or identical (over the length of the prove) to a plurality of nucleic acid, mRNA or miRNA molecules, precursor miRNA molecules, or nucleic acids derived from the various genes and gene pathways modulated by miR-200 miRNAs and that are positioned on a support or support material in a spatially separated organization. Macroarrays are typically sheets of nitrocellulose or nylon upon which probes have been spotted. Microarrays position the nucleic acid probes more densely such that up to 10,000 nucleic acid molecules can be fit into a region typically 1 to 4 square centimeters. Microarrays can be fabricated by spotting nucleic acid molecules, e.g., genes, oligonucleotides, etc., onto substrates or fabricating oligonucleotide sequences in situ on a substrate. Spotted or fabricated nucleic acid molecules can be applied in a high density matrix pattern of up to about 30 non-identical nucleic acid molecules per square centimeter or higher, e.g. up to about 100 or even 1000 per square centimeter. Microarrays typically use coated glass as the solid support, in contrast to the nitrocellulose-based material of filter arrays. By having an ordered array of marker RNA and/or miRNA-complementing nucleic acid samples, the position of each sample can be tracked and linked to the original sample.

A variety of different array devices in which a plurality of distinct nucleic acid probes are stably associated with the surface of a solid support are known to those of skill in the art. Useful substrates for arrays include nylon, glass, metal, plastic, latex, and silicon. Such arrays may vary in a number of different ways, including average probe length, sequence or types of probes, nature of bond between the probe and the array surface, e.g. covalent or non-covalent, and the like. The labeling and screening methods of the present invention and the arrays are not limited in its utility with respect to any parameter except that the probes detect miRNA, or genes or nucleic acid representative of genes; consequently, methods and compositions may be used with a variety of different types of nucleic acid arrays.

Representative methods and apparatus for preparing a microarray have been described, for example, in U.S. Pat. Nos. 5,143,854; 5,202,231; 5,242,974; 5,288,644; 5,324,633; 5,384,261; 5,405,783; 5,412,087; 5,424,186; 5,429,807; 5,432,049; 5,436,327; 5,445,934; 5,468,613; 5,470,710; 5,472,672; 5,492,806; 5,525,464; 5,503,980; 5,510,270; 5,525,464; 5,527,681; 5,529,756; 5,532,128; 5,545,531; 5,547,839; 5,554,501; 5,556,752; 5,561,071; 5,571,639; 5,580,726; 5,580,732; 5,593,839; 5,599,695; 5,599,672; 5,610,287; 5,624,711; 5,631,134; 5,639,603; 5,654,413; 5,658,734; 5,661,028; 5,665,547; 5,667,972; 5,695,940; 5,700,637; 5,744,305; 5,800,992; 5,807,522; 5,830,645; 5,837,196; 5,871,928; 5,847,219; 5,876,932; 5,919,626; 6,004,755; 6,087,102; 6,368,799; 6,383,749; 6,617,112; 6,638,717; 6,720,138, as well as WO 93/17126; WO 95/11995; WO 95/21265; WO 95/21944; WO 95/35505; WO 96/31622; WO 97/10365; WO 97/27317; WO 99/35505; WO 09923256; WO 09936760; WO0138580; WO 0168255; WO 03020898; WO 03040410; WO 03053586; WO 03087297; WO 03091426; WO03100012; WO 04020085; WO 04027093; EP 373203; EP 785 280; EP 799 897 and UK 8 803 000; the disclosures of which are all herein incorporated by reference.

It is contemplated that the arrays can be high density arrays, such that they contain 2, 20, 25, 50, 80, 100 or more different probes. It is contemplated that they may contain 1000, 16,000, 65,000, 250,000 or 1,000,000 or more different probes. The probes can be directed to mRNA and/or miRNA targets in one or more different organisms or cell types. The oligonucleotide probes range from 5 to 50, 5 to 45, 10 to 40, 9 to 34, or 15 to 40 nucleotides in length in some embodiments. In certain embodiments, the oligonucleotide probes are 5, 10, 15, to 20, 25, 30, 35, 40 nucleotides in length including all integers and ranges there between.

The location and sequence of each different probe sequence in the array are generally known. Moreover, the large number of different probes can occupy a relatively small area providing a high density array having a probe density of generally greater than about 60, 100, 600, 1000, 5,000, 10,000, 40,000, 100,000, or 400,000 different oligonucleotide probes per cm². The surface area of the array can be about or less than about 1, 1.6, 2, 3, 4, 5, 6, 7, 8, 9, or 10 cm².

Moreover, a person of ordinary skill in the art could readily analyze data generated using an array. Such protocols are disclosed above, and include information found in WO 9743450; WO 03023058; WO 03022421; WO 03029485; WO 03067217; WO 03066906; WO 03076928; WO 03093810; WO 03100448A1, all of which are specifically incorporated by reference.

B. Sample Preparation

It is contemplated that the RNA and/or miRNA of a wide variety of samples can be analyzed using the arrays, index of probes, or array technology of the invention. While endogenous miRNA is contemplated for use with compositions and methods of the invention, recombinant miRNA—including nucleic acids that are complementary or identical to endogenous miRNA or precursor miRNA—can also be handled and analyzed as described herein. Samples may be biological samples, in which case, they can be from biopsy, fine needle aspirates, exfoliates, blood, tissue, organs, semen, saliva, tears, other bodily fluid, hair follicles, skin, or any sample containing or constituting biological cells, particularly cancer or hyperproliferative cells. In certain embodiments, samples may be, but are not limited to, biopsy, or cells purified or enriched to some extent from a biopsy or other bodily fluids or tissues. Alternatively, the sample may not be a biological sample, but be a chemical mixture, such as a cell-free reaction mixture (which may contain one or more biological enzymes).

C. Hybridization

After an array or a set of probes is prepared and/or the nucleic acid in the sample or probe is labeled, the population of target nucleic acids is contacted with the array or probes under hybridization conditions, where such conditions can be adjusted, as desired, to provide for an optimum level of specificity in view of the particular assay being performed. Suitable hybridization conditions are well known to those of skill in the art and reviewed in Sambrook et al. (2001) and WO 95/21944. Of particular interest in many embodiments is the use of stringent conditions during hybridization. Stringent conditions are known to those of skill in the art.

It is specifically contemplated that a single array or set of probes may be contacted with multiple samples. The samples may be labeled with different labels to distinguish the samples. For example, a single array can be contacted with a tumor tissue sample labeled with Cy3, and normal tissue sample labeled with Cy5. Differences between the samples for particular miRNAs corresponding to probes on the array can be readily ascertained and quantified.

The small surface area of the array permits uniform hybridization conditions, such as temperature regulation and salt content. Moreover, because of the small area occupied by the high density arrays, hybridization may be carried out in extremely small fluid volumes (e.g., about 250 μl or less, including volumes of about or less than about 5, 10, 25, 50, 60, 70, 80, 90, 100 μl, or any range derivable therein). In small volumes, hybridization may proceed very rapidly.

D. Differential Expression Analyses

Arrays of the invention can be used to detect differences between two samples. Specifically contemplated applications include identifying and/or quantifying differences between miRNA or gene expression from a sample that is normal and from a sample that is not normal, between a disease or condition and a cell not exhibiting such a disease or condition, or between two differently treated samples. Also, miRNA or gene expression may be compared between a sample believed to be susceptible to a particular disease or condition and one believed to be not susceptible or resistant to that disease or condition. A sample that is not normal is one exhibiting phenotypic or genotypic trait(s) of a disease or condition, or one believed to be not normal with respect to that disease or condition. It may be compared to a cell that is normal with respect to that disease or condition. Phenotypic traits include symptoms of, or susceptibility to, a disease or condition of which a component is or may or may not be genetic, or caused by a hyperproliferative or neoplastic cell or cells.

An array comprises a solid support with nucleic acid probes attached to the support. Arrays typically comprise a plurality of different nucleic acid probes that are coupled to a surface of a substrate in different, known locations. These arrays, also described as “microarrays” or colloquially “chips” have been generally described in the art, for example, U.S. Pat. Nos. 5,143,854, 5,445,934, 5,744,305, 5,677,195, 6,040,193, 5,424,186 and Fodor et al., (1991), each of which is incorporated by reference in its entirety for all purposes. Techniques for the synthesis of these arrays using mechanical synthesis methods are described in, e.g., U.S. Pat. No. 5,384,261, incorporated herein by reference in its entirety for all purposes. Although a planar array surface is used in certain aspects, the array may be fabricated on a surface of virtually any shape or even a multiplicity of surfaces. Arrays may be nucleic acids on beads, gels, polymeric surfaces, fibers such as fiber optics, glass or any other appropriate substrate, see U.S. Pat. Nos. 5,770,358, 5,789,162, 5,708,153, 6,040,193 and 5,800,992, which are hereby incorporated in their entirety for all purposes. Arrays may be packaged in such a manner as to allow for diagnostics or other manipulation of an all inclusive device, see for example, U.S. Pat. Nos. 5,856,174 and 5,922,591 incorporated in their entirety by reference for all purposes. See also U.S. patent application Ser. No. 09/545,207, filed Apr. 7, 2000 for additional information concerning arrays, their manufacture, and their characteristics, which is incorporated by reference in its entirety for all purposes.

Particularly, arrays can be used to evaluate samples with respect to pathological condition such as cancer and related conditions. It is specifically contemplated that the invention can be used to evaluate differences between stages or sub-classifications of disease, such as between benign, cancerous, and metastatic tissues or tumors.

Phenotypic traits to be assessed include characteristics such as longevity, morbidity, expected survival, susceptibility or receptivity to particular drugs or therapeutic treatments (drug efficacy), and risk of drug toxicity. Samples that differ in these phenotypic traits may also be evaluated using the compositions and methods described.

In certain embodiments, miRNA and/or expression profiles may be generated to evaluate and correlate those profiles with pharmacokinetics or therapies. For example, these profiles may be created and evaluated for patient tumor and blood samples prior to the patient's being treated or during treatment to determine if there are miRNA or genes whose expression correlates with the outcome of the patient's treatment. Identification of differential miRNAs or genes can lead to a diagnostic assay for evaluation of tumor and/or blood samples to determine what drug regimen the patient should be provided. In addition, it can be used to identify or select patients suitable for a particular clinical trial. If an expression profile is determined to be correlated with drug efficacy or drug toxicity that profile is relevant to whether that patient is an appropriate patient for receiving a drug, for receiving a combination of drugs, or for a particular dosage of the drug.

In addition to the above prognostic assay, samples from patients with a variety of diseases can be evaluated to determine if different diseases can be identified based on miRNA and/or related gene expression levels. A diagnostic assay can be created based on the profiles that doctors can use to identify individuals with a disease or who are at risk to develop a disease. Alternatively, treatments can be designed based on miRNA profiling. Examples of such methods and compositions are described in the U.S. Provisional Patent Application entitled “Methods and Compositions Involving miRNA and miRNA Inhibitor Molecules” filed on May 23, 2005, which is hereby incorporated by reference in its entirety.

E. Other Assays

In addition to the use of arrays and microarrays, it is contemplated that a number of different assays could be employed to analyze miRNAs or related genes, their activities, and their effects. Such assays include, but are not limited to, nucleic acid amplification, polymerase chain reaction, quantitative PCR, RT-PCR, in situ hybridization, Northern hybridization, hybridization protection assay (HPA)(GenProbe), branched DNA (bDNA) assay (Chiron), rolling circle amplification (RCA), single molecule hybridization detection (US Genomics), Invader assay (ThirdWave Technologies), and/or Bridge Litigation Assay (Genaco).

### IV. Nucleic Acids

The present invention concerns nucleic acids, modified or mimetic nucleic acids, miRNAs, mRNAs, genes, and representative fragments thereof that can be labeled, used in array analysis, or employed in diagnostic, therapeutic, or prognostic applications, particularly those related to pathological conditions such as cancer. The molecules may have been endogenously produced by a cell, or been synthesized or produced chemically or recombinantly. They may be isolated and/or purified. Each of the miRNAs described herein and include the corresponding SEQ ID NO and accession numbers for these miRNA sequences. The name of a miRNA is often abbreviated and referred to without a “hsa-” prefix and will be understood as such, depending on the context. Unless otherwise indicated, miRNAs referred to in the application are human sequences identified as miR-X or let-X, where X is a number and/or letter.

In certain aspects, a miRNA probe designated by a suffix “5P” or “3P” can be used. “5P” indicates that the mature miRNA derives from the 5′ end of the precursor and a corresponding “3P” indicates that it derives from the 3′ end of the precursor, as described on the world wide web at sanger.ac.uk. Moreover, in some embodiments, a miRNA probe is used that does not correspond to a known human miRNA. It is contemplated that these non-human miRNA probes may be used in embodiments of the invention or that there may exist a human miRNA that is homologous to the non-human miRNA. In other embodiments, any mammalian cell, biological sample, or preparation thereof may be employed.

In some embodiments of the invention, methods and compositions involving miRNA may concern miRNA, markers (mRNAs), and/or other nucleic acids. Nucleic acids may be, be at least, or be at most 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, or 1000 nucleotides, or any range derivable therein, in length. Such lengths cover the lengths of processed miRNA, miRNA probes, precursor miRNA, miRNA containing vectors, mRNA, mRNA probes, control nucleic acids, and other probes and primers.

In many embodiments, miRNA are 19-24 nucleotides in length, while miRNA probes are 19-35 nucleotides in length, depending on the length of the processed miRNA and any flanking regions added. miRNA precursors are generally between 62 and 110 nucleotides in humans.

Nucleic acids of the invention may have regions of identity or complementarity to another nucleic acid. It is contemplated that the region of complementarity or identity can be at least 5 contiguous residues, though it is specifically contemplated that the region is, is at least, or is at most 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 441, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, or 1000 contiguous nucleotides. It is further understood that the length of complementarity within a precursor miRNA or other nucleic acid or between a miRNA probe and a miRNA or a miRNA gene are such lengths. Moreover, the complementarity may be expressed as a percentage, meaning that the complementarity between a probe and its target is 90% or greater over the length of the probe. In some embodiments, complementarity is or is at least 90%, 95% or 100%. In particular, such lengths may be applied to any nucleic acid comprising a nucleic acid sequence identified in any of SEQ ID NOs described herein, accession number, or any other sequence disclosed herein. Typically, the commonly used name of the miRNA is given (with its identifying source in the prefix, for example, “hsa” for human sequences) and the processed miRNA sequence. Unless otherwise indicated, a miRNA without a prefix will be understood to refer to a human miRNA. Moreover, a lowercase letter in a miRNA name may or may not be lowercase; for example, hsa-mir-130b can also be referred to as miR-130B. The term “miRNA probe” refers to a nucleic acid probe that can identify a particular miRNA or structurally related miRNAs.

It is understood that some nucleic acids are derived from genomic sequences or a gene. In this respect, the term “gene” is used for simplicity to refer to the genomic sequence encoding the precursor nucleic acid or miRNA for a given miRNA or gene. However, embodiments of the invention may involve genomic sequences of a miRNA that are involved in its expression, such as a promoter or other regulatory sequences.

The term “recombinant” may be used and this generally refers to a molecule that has been manipulated in vitro or that is a replicated or expressed product of such a molecule.

The term “nucleic acid” is well known in the art. A “nucleic acid” as used herein will generally refer to a molecule (one or more strands) of DNA, RNA or a derivative or analog thereof, comprising a nucleobase. A nucleobase includes, for example, a naturally occurring purine or pyrimidine base found in DNA (e.g., an adenine “A,” a guanine “G,” a thymine “T” or a cytosine “C”) or RNA (e.g., an A, a G, an uracil “U” or a C). The term “nucleic acid” encompasses the terms “oligonucleotide” and “polynucleotide,” each as a subgenus of the term “nucleic acid.”

The term “miRNA” generally refers to a single-stranded molecule, but in specific embodiments, molecules implemented in the invention will also encompass a region or an additional strand that is partially (between 10 and 50% complementary across length of strand), substantially (greater than 50% but less than 100% complementary across length of strand) or fully complementary to another region of the same single-stranded molecule or to another nucleic acid. Thus, miRNA may encompass a molecule that comprises one or more complementary or self-complementary strand(s) or “complement(s)” of a particular sequence. For example, precursor miRNA may have a self-complementary region, which is up to 100% complementary. miRNA probes or nucleic acids of the invention can include, can be or can be at least 60, 65, 70, 75, 80, 85, 90, 95, 96, 97, 98, 99 or 100% complementary to their target.

It is understood that a “synthetic nucleic acid” of the invention means that the nucleic acid does not have all or part of a chemical structure or sequence of a naturally occurring nucleic acid. Consequently, it will be understood that the term “synthetic miRNA” refers to a “synthetic nucleic acid” that functions in a cell or under physiological conditions as a naturally occurring miRNA.

While embodiments of the invention may involve synthetic miRNAs or synthetic nucleic acids, in some embodiments of the invention, the nucleic acid molecule(s) need not be “synthetic.” In certain embodiments, a non-synthetic nucleic acid or miRNA employed in methods and compositions of the invention may have the entire sequence and structure of a naturally occurring mRNA or miRNA precursor or the mature mRNA or miRNA. For example, non-synthetic miRNAs used in methods and compositions of the invention may not have one or more modified nucleotides or nucleotide analogs. In these embodiments, the non-synthetic miRNA may or may not be recombinantly produced. In particular embodiments, the nucleic acid in methods and/or compositions of the invention is specifically a synthetic miRNA and not a non-synthetic miRNA (that is, not an miRNA that qualifies as “synthetic”); though in other embodiments, the invention specifically involves a non-synthetic miRNA and not a synthetic miRNA. Any embodiments discussed with respect to the use of synthetic miRNAs can be applied with respect to non-synthetic miRNAs, and vice versa.

It will be understood that the term “naturally occurring” refers to something found in an organism without any intervention by a person; it could refer to a naturally-occurring wildtype or mutant molecule. In some embodiments a synthetic miRNA molecule does not have the sequence of a naturally occurring miRNA molecule. In other embodiments, a synthetic miRNA molecule may have the sequence of a naturally occurring miRNA molecule, but the chemical structure of the molecule, particularly in the part unrelated specifically to the precise sequence (non-sequence chemical structure) differs from chemical structure of the naturally occurring miRNA molecule with that sequence. In some cases, the synthetic miRNA has both a sequence and non-sequence chemical structure that are not found in a naturally-occurring miRNA. Moreover, the sequence of the synthetic molecules will identify which miRNA is effectively being provided or inhibited; the endogenous miRNA will be referred to as the “corresponding miRNA.” Corresponding miRNA sequences that can be used in the context of the invention include, but are not limited to, all or a portion of those sequences in the SEQ IDs provided herein, as well as any other miRNA sequence, miRNA precursor sequence, or any sequence complementary thereof. In some embodiments, the sequence is or is derived from or contains all or part of a sequence identified herein to target a particular miRNA (or set of miRNAs) that can be used with that sequence. Any 1, 2, 3, 4, 5, 6, 7, 8, 9, 10, 11, 12, 13, 14, 15, 16, 17, 18, 19, 20, 30, 40, 50, 60, 70, 80, 90, 100, 110, 120, 130 140, 150, 160, 170, 180, 190, 200, 210, 220, 230, 240, 250, 260 or any number or range of sequences there between may be selected to the exclusion of all non-selected sequences.

As used herein, “hybridization”, “hybridizes” or “capable of hybridizing” is understood to mean the forming of a double or triple stranded molecule or a molecule with partial double or triple stranded nature. The term “anneal” as used herein is synonymous with “hybridize.” The term “hybridization”, “hybridize(s)” or “capable of hybridizing” encompasses the terms “stringent condition(s)” or “high stringency” and the terms “low stringency” or “low stringency condition(s).”

As used herein “stringent condition(s)” or “high stringency” are those conditions that allow hybridization between or within one or more nucleic acid strand(s) containing complementary sequence(s), but preclude hybridization of random sequences. Stringent conditions tolerate little, if any, mismatch between a nucleic acid and a target strand. Such conditions are well known to those of ordinary skill in the art, and are preferred for applications requiring high selectivity. Non-limiting applications include isolating a nucleic acid, such as a gene or a nucleic acid segment thereof, or detecting at least one specific mRNA transcript or a nucleic acid segment thereof, and the like.

Stringent conditions may comprise low salt and/or high temperature conditions, such as provided by about 0.02 M to about 0.5 M NaCl at temperatures of about 42° C. to about 70° C. It is understood that the temperature and ionic strength of a desired stringency are determined in part by the length of the particular nucleic acid(s), the length and nucleobase content of the target sequence(s), the charge composition of the nucleic acid(s), and to the presence or concentration of formamide, tetramethylammonium chloride or other solvent(s) in a hybridization mixture.

It is also understood that these ranges, compositions and conditions for hybridization are mentioned by way of non-limiting examples only, and that the desired stringency for a particular hybridization reaction is often determined empirically by comparison to one or more positive or negative controls. Depending on the application envisioned it is preferred to employ varying conditions of hybridization to achieve varying degrees of selectivity of a nucleic acid towards a target sequence. In a non-limiting example, identification of a related target nucleic acid that does not hybridize to a nucleic acid under stringent conditions may be achieved by hybridization at low temperature and/or high ionic strength. Such conditions are termed “low stringency” or “low stringency conditions,” and non-limiting examples of low stringency include hybridization performed at about 0.15 M to about 0.9 M NaCl at a temperature range of about 20° C. to about 50° C. Of course, it is within the skill of one in the art to further modify the low or high stringency conditions to suite a particular application.

A. Nucleobase, Nucleoside, Nucleotide, and Modified Nucleotides

As used herein a “nucleobase” refers to a heterocyclic base, such as for example a naturally occurring nucleobase (i.e., an A, T, G, C or U) found in at least one naturally occurring nucleic acid (i.e., DNA and RNA), and naturally or non-naturally occurring derivative(s) and analogs of such a nucleobase. A nucleobase generally can form one or more hydrogen bonds (“anneal” or “hybridize”) with at least one naturally occurring nucleobase in a manner that may substitute for naturally occurring nucleobase pairing (e.g., the hydrogen bonding between A and T, G and C, and A and U).

“Purine” and/or “pyrimidine” nucleobase(s) encompass naturally occurring purine and/or pyrimidine nucleobases and also derivative(s) and analog(s) thereof, including but not limited to, those a purine or pyrimidine substituted by one or more of an alkyl, caboxyalkyl, amino, hydroxyl, halogen (i.e., fluoro, chloro, bromo, or iodo), thiol or alkylthiol moiety. Preferred alkyl (e.g., alkyl, caboxyalkyl, etc.) moieties comprise of from about 1, about 2, about 3, about 4, about 5, to about 6 carbon atoms. Other non-limiting examples of a purine or pyrimidine include a deazapurine, a 2,6-diaminopurine, a 5-fluorouracil, a xanthine, a hypoxanthine, a 8-bromoguanine, a 8-chloroguanine, a bromothymine, a 8-aminoguanine, a 8-hydroxyguanine, a 8-methylguanine, a 8-thioguanine, an azaguanine, a 2-aminopurine, a 5-ethylcytosine, a 5-methylcyosine, a 5-bromouracil, a 5-ethyluracil, a 5-iodouracil, a 5-chlorouracil, a 5-propyluracil, a thiouracil, a 2-methyladenine, a methylthioadenine, a N,N-diemethyladenine, an azaadenines, a 8-bromoadenine, a 8-hydroxyadenine, a 6-hydroxyaminopurine, a 6-thiopurine, a 4-(6-aminohexyl/cytosine), and the like. Other examples are well known to those of skill in the art.

As used herein, a “nucleoside” refers to an individual chemical unit comprising a nucleobase covalently attached to a nucleobase linker moiety. A non-limiting example of a “nucleobase linker moiety” is a sugar comprising 5-carbon atoms (i.e., a “5-carbon sugar”), including but not limited to a deoxyribose, a ribose, an arabinose, or a derivative or an analog of a 5-carbon sugar. Non-limiting examples of a derivative or an analog of a 5-carbon sugar include a 2′-fluoro-2′-deoxyribose or a carbocyclic sugar where a carbon is substituted for an oxygen atom in the sugar ring. Different types of covalent attachment(s) of a nucleobase to a nucleobase linker moiety are known in the art (Kornberg and Baker, 1992).

As used herein, a “nucleotide” refers to a nucleoside further comprising a “backbone moiety”. A backbone moiety generally covalently attaches a nucleotide to another molecule comprising a nucleotide, or to another nucleotide to form a nucleic acid. The “backbone moiety” in naturally occurring nucleotides typically comprises a phosphorus moiety, which is covalently attached to a 5-carbon sugar. The attachment of the backbone moiety typically occurs at either the 3′- or 5′-position of the 5-carbon sugar. However, other types of attachments are known in the art, particularly when a nucleotide comprises derivatives or analogs of a naturally occurring 5-carbon sugar or phosphorus moiety.

A nucleic acid may comprise, or be composed entirely of, a derivative or analog of a nucleobase, a nucleobase linker moiety and/or backbone moiety that may be present in a naturally occurring nucleic acid. RNA with nucleic acid analogs may also be labeled according to methods of the invention. As used herein a “derivative” refers to a chemically modified or altered form of a naturally occurring molecule, while the terms “mimic” or “analog” refer to a molecule that may or may not structurally resemble a naturally occurring molecule or moiety, but possesses similar functions. As used herein, a “moiety” generally refers to a smaller chemical or molecular component of a larger chemical or molecular structure. Nucleobase, nucleoside and nucleotide analogs or derivatives are well known in the art, and have been described (see for example, Scheit, 1980, incorporated herein by reference).

Additional non-limiting examples of nucleosides, nucleotides or nucleic acids include those in: U.S. Pat. Nos. 5,681,947, 5,652,099 and 5,763,167, 5,614,617, 5,670,663, 5,872,232, 5,859,221, 5,446,137, 5,886,165, 5,714,606, 5,672,697, 5,466,786, 5,792,847, 5,223,618, 5,470,967, 5,378,825, 5,777,092, 5,623,070, 5,610,289, 5,602,240, 5,858,988, 5,214,136, 5,700,922, 5,708,154, 5,728,525, 5,637,683, 6,251,666, 5,480,980, and 5,728,525, each of which is incorporated herein by reference in its entirety.

Labeling methods and kits of the invention specifically contemplate the use of nucleotides that are both modified for attachment of a label and can be incorporated into a miRNA molecule. Such nucleotides include those that can be labeled with a dye, including a fluorescent dye, or with a molecule such as biotin. Labeled nucleotides are readily available; they can be acquired commercially or they can be synthesized by reactions known to those of skill in the art.

Modified nucleotides for use in the invention are not naturally occurring nucleotides, but instead, refer to prepared nucleotides that have a reactive moiety on them. Specific reactive functionalities of interest include: amino, sulfhydryl, sulfoxyl, aminosulfhydryl, azido, epoxide, isothiocyanate, isocyanate, anhydride, monochlorotriazine, dichlorotriazine, mono- or dihalogen substituted pyridine, mono- or disubstituted diazine, maleimide, epoxide, aziridine, sulfonyl halide, acid halide, alkyl halide, aryl halide, alkylsulfonate, N-hydroxysuccinimide ester, imido ester, hydrazine, azidonitrophenyl, azide, 3-(2-pyridyl dithio)-propionamide, glyoxal, aldehyde, iodoacetyl, cyanomethyl ester, p-nitrophenyl ester, o-nitrophenyl ester, hydroxypyridine ester, carbonyl imidazole, and the other such chemical groups. In some embodiments, the reactive functionality may be bonded directly to a nucleotide, or it may be bonded to the nucleotide through a linking group. The functional moiety and any linker cannot substantially impair the ability of the nucleotide to be added to the miRNA or to be labeled. Representative linking groups include carbon containing linking groups, typically ranging from about 2 to 18, usually from about 2 to 8 carbon atoms, where the carbon containing linking groups may or may not include one or more heteroatoms, e.g. S, O, N etc., and may or may not include one or more sites of unsaturation. Of particular interest in many embodiments are alkyl linking groups, typically lower alkyl linking groups of 1 to 16, usually 1 to 4 carbon atoms, where the linking groups may include one or more sites of unsaturation. The functionalized nucleotides (or primers) used in the above methods of functionalized target generation may be fabricated using known protocols or purchased from commercial vendors, e.g., Sigma, Roche, Ambion, Biosearch Technologies and NEN. Functional groups may be prepared according to ways known to those of skill in the art, including the representative information found in U.S. Pat. Nos. 4,404,289; 4,405,711; 4,337,063 and 5,268,486, and U.K. Patent 1,529,202, which are all incorporated by reference.

Amine-modified nucleotides are used in several embodiments of the invention. The amine-modified nucleotide is a nucleotide that has a reactive amine group for attachment of the label. It is contemplated that any ribonucleotide (G, A, U, or C) or deoxyribonucleotide (G, A, T, or C) can be modified for labeling. Examples include, but are not limited to, the following modified ribo- and deoxyribo-nucleotides: 5-(3-aminoallyl)-UTP; 8-[(4-amino)butyl]-amino-ATP and 8-[(6-amino)butyl]-amino-ATP; N6-(4-amino)butyl-ATP, N6-(6-amino)butyl-ATP, N4-[2,2-oxy-bis-(ethylamine)]-CTP; N6-(6-Amino)hexyl-ATP; 8-[(6-Amino)hexyl]-amino-ATP; 5-propargylamino-CTP, 5-propargylamino-UTP; 5-(3-aminoallyl)-dUTP; 8-[(4-amino)butyl]-amino-dATP and 8-[(6-amino)butyl]-amino-dATP; N6-(4-amino)butyl-dATP, N6-(6-amino)butyl-dATP, N4-[2,2-oxy-bis-(ethylamine)]-dCTP; N6-(6-Amino)hexyl-dATP; 8-[(6-Amino)hexyl]-amino-dATP; 5-propargylamino-dCTP, and 5-propargylamino-dUTP. Such nucleotides can be prepared according to methods known to those of skill in the art. Moreover, a person of ordinary skill in the art could prepare other nucleotide entities with the same amine-modification, such as a 5-(3-aminoallyl)-CTP, GTP, ATP, dCTP, dGTP, dTTP, or dUTP in place of a 5-(3-aminoallyl)-UTP.

B. Preparation of Nucleic Acids

A nucleic acid may be made by any technique known to one of ordinary skill in the art, such as for example, chemical synthesis, enzymatic production, or biological production. It is specifically contemplated that miRNA probes of the invention are chemically synthesized.

In some embodiments of the invention, miRNAs are recovered or isolated from a biological sample. The miRNA may be recombinant or it may be natural or endogenous to the cell (produced from the cell's genome). It is contemplated that a biological sample may be treated in a way so as to enhance the recovery of small RNA molecules such as miRNA. U.S. patent application Ser. No. 10/667,126 describes such methods and it is specifically incorporated by reference herein. Generally, methods involve lysing cells with a solution having guanidinium and a detergent.

Alternatively, nucleic acid synthesis is performed according to standard methods. See, for example, Itakura and Riggs (1980) and U.S. Pat. Nos. 4,704,362, 5,221,619, and 5,583,013, each of which is incorporated herein by reference. Non-limiting examples of a synthetic nucleic acid (e.g., a synthetic oligonucleotide), include a nucleic acid made by in vitro chemically synthesis using phosphotriester, phosphite, or phosphoramidite chemistry and solid phase techniques such as described in EP 266,032, incorporated herein by reference, or via deoxynucleoside H-phosphonate intermediates as described by Froehler et al., 1986 and U.S. Pat. No. 5,705,629, each incorporated herein by reference. Various different mechanisms of oligonucleotide synthesis have been disclosed in for example, U.S. Pat. Nos. 4,659,774, 4,816,571, 5,141,813, 5,264,566, 4,959,463, 5,428,148, 5,554,744, 5,574,146, 5,602,244, each of which is incorporated herein by reference.

A non-limiting example of an enzymatically produced nucleic acid include one produced by enzymes in amplification reactions such as PCR™ (see for example, U.S. Pat. Nos. 4,683,202 and 4,682,195, each incorporated herein by reference), or the synthesis of an oligonucleotide described in U.S. Pat. No. 5,645,897, incorporated herein by reference. See also Sambrook et al., 2001, incorporated herein by reference).

Oligonucleotide synthesis is well known to those of skill in the art. Various different mechanisms of oligonucleotide synthesis have been disclosed in for example, U.S. Pat. Nos. 4,659,774, 4,816,571, 5,141,813, 5,264,566, 4,959,463, 5,428,148, 5,554,744, 5,574,146, 5,602,244, each of which is incorporated herein by reference.

Recombinant methods for producing nucleic acids in a cell are well known to those of skill in the art. These include the use of vectors (viral and non-viral), plasmids, cosmids, and other vehicles for delivering a nucleic acid to a cell, which may be the target cell (e.g., a cancer cell) or simply a host cell (to produce large quantities of the desired RNA molecule). Alternatively, such vehicles can be used in the context of a cell free system so long as the reagents for generating the RNA molecule are present. Such methods include those described in Sambrook, 2003, Sambrook, 2001 and Sambrook, 1989, which are hereby incorporated by reference.

C. Isolation of Nucleic Acids

Nucleic acids may be isolated using techniques well known to those of skill in the art, though in particular embodiments, methods for isolating small nucleic acid molecules, and/or isolating RNA molecules can be employed. Chromatography is a process often used to separate or isolate nucleic acids from protein or from other nucleic acids. Such methods can involve electrophoresis with a gel matrix, filter columns, alcohol precipitation, and/or other chromatography. If miRNA from cells is to be used or evaluated, methods generally involve lysing the cells with a chaotropic (e.g., guanidinium isothiocyanate) and/or detergent (e.g., N-lauroyl sarcosine) prior to implementing processes for isolating particular populations of RNA.

In particular methods for separating miRNA from other nucleic acids, a gel matrix is prepared using polyacrylamide, though agarose can also be used. The gels may be graded by concentration or they may be uniform. Plates or tubing can be used to hold the gel matrix for electrophoresis. Usually one-dimensional electrophoresis is employed for the separation of nucleic acids. Plates are used to prepare a slab gel, while the tubing (glass or rubber, typically) can be used to prepare a tube gel. The phrase “tube electrophoresis” refers to the use of a tube or tubing, instead of plates, to form the gel. Materials for implementing tube electrophoresis can be readily prepared by a person of skill in the art or purchased, such as from C.B.S. Scientific Co., Inc. or Scie-Plas.

Methods may involve the use of organic solvents and/or alcohol to isolate nucleic acids, particularly miRNA used in methods and compositions of the invention. Some embodiments are described in U.S. patent application Ser. No. 10/667,126, which is hereby incorporated by reference. Generally, this disclosure provides methods for efficiently isolating small RNA molecules from cells comprising: adding an alcohol solution to a cell lysate and applying the alcohol/lysate mixture to a solid support before eluting the RNA molecules from the solid support. In some embodiments, the amount of alcohol added to a cell lysate achieves an alcohol concentration of about 55% to 60%. While different alcohols can be employed, ethanol works well. A solid support may be any structure, and it includes beads, filters, and columns, which may include a mineral or polymer support with electronegative groups. A glass fiber filter or column has worked particularly well for such isolation procedures.

In specific embodiments, miRNA isolation processes include: a) lysing cells in the sample with a lysing solution comprising guanidinium, wherein a lysate with a concentration of at least about 1 M guanidinium is produced; b) extracting miRNA molecules from the lysate with an extraction solution comprising phenol; c) adding to the lysate an alcohol solution for forming a lysate/alcohol mixture, wherein the concentration of alcohol in the mixture is between about 35% to about 70%; d) applying the lysate/alcohol mixture to a solid support; e) eluting the miRNA molecules from the solid support with an ionic solution; and, f) capturing the miRNA molecules. Typically the sample is dried and resuspended in a liquid and volume appropriate for subsequent manipulation.

### V. Labels and Labeling Techniques

In some embodiments, the present invention concerns miRNA that are labeled. It is contemplated that miRNA may first be isolated and/or purified prior to labeling. This may achieve a reaction that more efficiently labels the miRNA, as opposed to other RNA in a sample in which the miRNA is not isolated or purified prior to labeling. In many embodiments of the invention, the label is non-radioactive. Generally, nucleic acids may be labeled by adding labeled nucleotides (one-step process) or adding nucleotides and labeling the added nucleotides (two-step process).

A. Labeling Techniques

In some embodiments, nucleic acids are labeled by catalytically adding to the nucleic acid an already labeled nucleotide or nucleotides. One or more labeled nucleotides can be added to miRNA molecules. See U.S. Pat. No. 6,723,509, which is hereby incorporated by reference.

In other embodiments, an unlabeled nucleotide or nucleotides is catalytically added to a miRNA, and the unlabeled nucleotide is modified with a chemical moiety that enables it to be subsequently labeled. In embodiments of the invention, the chemical moiety is a reactive amine such that the nucleotide is an amine-modified nucleotide. Examples of amine-modified nucleotides are well known to those of skill in the art, many being commercially available such as from Ambion, Sigma, Jena Bioscience, and TriLink.

In contrast to labeling of cDNA during its synthesis, the issue for labeling miRNA is how to label the already existing molecule. The present invention concerns the use of an enzyme capable of using a di- or tri-phosphate ribonucleotide or deoxyribonucleotide as a substrate for its addition to a miRNA. Moreover, in specific embodiments, it involves using a modified di- or tri-phosphate ribonucleotide, which is added to the 3′ end of a miRNA. Enzymes capable of adding such nucleotides include, but are not limited to, poly(A) polymerase, terminal transferase, and polynucleotide phosphorylase. In specific embodiments of the invention, a ligase is contemplated as not being the enzyme used to add the label, and instead, a non-ligase enzyme is employed. Terminal transferase catalyzes the addition of nucleotides to the 3′ terminus of a nucleic acid. Polynucleotide phosphorylase can polymerize nucleotide diphosphates without the need for a primer.

B. Labels

Labels on miRNA or miRNA probes may be colorimetric (includes visible and UV spectrum, including fluorescent), luminescent, enzymatic, or positron emitting (including radioactive). The label may be detected directly or indirectly. Radioactive labels include ¹²⁵I, ³²P, ³³P, and ³⁵S. Examples of enzymatic labels include alkaline phosphatase, luciferase, horseradish peroxidase, and β-galactosidase. Labels can also be proteins with luminescent properties, e.g., green fluorescent protein and phycoerythrin.

The colorimetric and fluorescent labels contemplated for use as conjugates include, but are not limited to, Alexa Fluor dyes, BODIPY dyes, such as BODIPY FL; Cascade Blue; Cascade Yellow; coumarin and its derivatives, such as 7-amino-4-methylcoumarin, aminocoumarin and hydroxycoumarin; cyanine dyes, such as Cy3 and Cy5; eosins and erythrosins; fluorescein and its derivatives, such as fluorescein isothiocyanate; macrocyclic chelates of lanthanide ions, such as Quantum Dye™; Marina Blue; Oregon Green; rhodamine dyes, such as rhodamine red, tetramethylrhodamine and rhodamine 6G; Texas Red; fluorescent energy transfer dyes, such as thiazole orange-ethidium heterodimer; and, TOTAB.

Specific examples of dyes include, but are not limited to, those identified above and the following: Alexa Fluor 350, Alexa Fluor 405, Alexa Fluor 430, Alexa Fluor 488, Alexa Fluor 500. Alexa Fluor 514, Alexa Fluor 532, Alexa Fluor 546, Alexa Fluor 555, Alexa Fluor 568, Alexa Fluor 594, Alexa Fluor 610, Alexa Fluor 633, Alexa Fluor 647, Alexa Fluor 660, Alexa Fluor 680, Alexa Fluor 700, and, Alexa Fluor 750; amine-reactive BODIPY dyes, such as BODIPY 493/503, BODIPY 530/550, BODIPY 558/568, BODIPY 564/570, BODIPY 576/589, BODIPY 581/591, BODIPY 630/650, BODIPY 650/655, BODIPY FL, BODIPY R6G, BODIPY TMR, and, BODIPY-TR; Cy3, Cy5, 6-FAM, Fluorescein Isothiocyanate, HEX, 6-JOE, Oregon Green 488, Oregon Green 500, Oregon Green 514, Pacific Blue, REG, Rhodamine Green, Rhodamine Red, Renographin, ROX, SYPRO, TAMRA, 2′,4′,5′,7′-Tetrabromosulfonefluorescein, and TET.

Specific examples of fluorescently labeled ribonucleotides are available from Molecular Probes, and these include, Alexa Fluor 488-5-UTP, Fluorescein-12-UTP, BODIPY FL-14-UTP, BODIPY TMR-14-UTP, Tetramethylrhodamine-6-UTP, Alexa Fluor 546-14-UTP, Texas Red-5-UTP, and BODIPY TR-14-UTP. Other fluorescent ribonucleotides are available from Amersham Biosciences, such as Cy3-UTP and Cy5-UTP.

Examples of fluorescently labeled deoxyribonucleotides include Dinitrophenyl (DNP)-1-dUTP, Cascade Blue-7-dUTP, Alexa Fluor 488-5-dUTP, Fluorescein-12-dUTP, Oregon Green 488-5-dUTP, BODIPY FL-14-dUTP, Rhodamine Green-5-dUTP, Alexa Fluor 532-5-dUTP, BODIPY TMR-14-dUTP, Tetramethylrhodamine-6-dUTP, Alexa Fluor 546-14-dUTP, Alexa Fluor 568-5-dUTP, Texas Red-12-dUTP, Texas Red-5-dUTP, BODIPY TR-14-dUTP, Alexa Fluor 594-5-dUTP, BODIPY 630/650-14-dUTP, BODIPY 650/665-14-dUTP; Alexa Fluor 488-7-OBEA-dCTP, Alexa Fluor 546-16-OBEA-dCTP, Alexa Fluor 594-7-OBEA-dCTP, Alexa Fluor 647-12-OBEA-dCTP.

It is contemplated that nucleic acids may be labeled with two different labels. Furthermore, fluorescence resonance energy transfer (FRET) may be employed in methods of the invention (e.g., Klostermeier et al., 2002; Emptage, 2001; Didenko, 2001, each incorporated by reference).

Alternatively, the label may not be detectable per se, but indirectly detectable or allowing for the isolation or separation of the targeted nucleic acid. For example, the label could be biotin, digoxigenin, polyvalent cations, chelator groups and the other ligands, include ligands for an antibody.

C. Visualization Techniques

A number of techniques for visualizing or detecting labeled nucleic acids are readily available. Such techniques include, microscopy, arrays, Fluorometry, Light cyclers or other real time PCR machines, FACS analysis, scintillation counters, Phosphoimagers, Geiger counters, MRI, CAT, antibody-based detection methods (Westerns, immunofluorescence, immunohistochemistry), histochemical techniques, HPLC (Griffey et al., 1997), spectroscopy, capillary gel electrophoresis (Cummins et al., 1996), spectroscopy; mass spectroscopy; radiological techniques; and mass balance techniques.

When two or more differentially colored labels are employed, fluorescent resonance energy transfer (FRET) techniques may be employed to characterize association of one or more nucleic acid. Furthermore, a person of ordinary skill in the art is well aware of ways of visualizing, identifying, and characterizing labeled nucleic acids, and accordingly, such protocols may be used as part of the invention. Examples of tools that may be used also include fluorescent microscopy, a BioAnalyzer, a plate reader, Storm (Molecular Dynamics), Array Scanner, FACS (fluorescent activated cell sorter), or any instrument that has the ability to excite and detect a fluorescent molecule.

### VI. KITS

Any of the compositions described herein may be comprised in a kit. In a non-limiting example, reagents for isolating miRNA, labeling miRNA, and/or evaluating a miRNA population using an array, nucleic acid amplification, and/or hybridization can be included in a kit, as well reagents for preparation of samples from blood samples. The kit may further include reagents for creating or synthesizing miRNA probes. The kits will thus comprise, in suitable container means, an enzyme for labeling the miRNA by incorporating labeled nucleotide or unlabeled nucleotides that are subsequently labeled. In certain aspects, the kit can include amplification reagents. In other aspects, the kit may include various supports, such as glass, nylon, polymeric beads, and the like, and/or reagents for coupling any probes and/or target nucleic acids. It may also include one or more buffers, such as reaction buffer, labeling buffer, washing buffer, or a hybridization buffer, compounds for preparing the miRNA probes, and components for isolating miRNA. Other kits of the invention may include components for making a nucleic acid array comprising miRNA, and thus, may include, for example, a solid support.

Kits for implementing methods of the invention described herein are specifically contemplated. In some embodiments, there are kits for preparing miRNA for multi-labeling and kits for preparing miRNA probes and/or miRNA arrays. In these embodiments, kit comprise, in suitable container means, 1, 2, 3, 4, 5, 6, 7, 8, 9, 10, 11, 12 or more of the following: (1) poly(A) polymerase; (2) unmodified nucleotides (G, A, T, C, and/or U); (3) a modified nucleotide (labeled or unlabeled); (4) poly(A) polymerase buffer; and, (5) at least one microfilter; (6) label that can be attached to a nucleotide; (7) at least one miRNA probe; (8) reaction buffer; (9) a miRNA array or components for making such an array; (10) acetic acid; (11) alcohol; (12) solutions for preparing, isolating, enriching, and purifying miRNAs or miRNA probes or arrays. Other reagents include those generally used for manipulating RNA, such as formamide, loading dye, ribonuclease inhibitors, and DNase.

In specific embodiments, kits of the invention include an array containing miRNA probes, as described in the application. An array may have probes corresponding to all known miRNAs of an organism or a particular tissue or organ in particular conditions, or to a subset of such probes. The subset of probes on arrays of the invention may be or include those identified as relevant to a particular diagnostic, therapeutic, or prognostic application. For example, the array may contain one or more probes that is indicative or suggestive of (1) a disease or condition (acute myeloid leukemia), (2) susceptibility or resistance to a particular drug or treatment; (3) susceptibility to toxicity from a drug or substance; (4) the stage of development or severity of a disease or condition (prognosis); and (5) genetic predisposition to a disease or condition.

For any kit embodiment, including an array, there can be nucleic acid molecules that contain or can be used to amplify a sequence that is a variant of, identical to or complementary to all or part of any of SEQ IDs described herein. In certain embodiments, a kit or array of the invention can contain one or more probes for the miRNAs identified by the SEQ IDs described herein. Any nucleic acid discussed above may be implemented as part of a kit.

The components of the kits may be packaged either in aqueous media or in lyophilized form. The container means of the kits will generally include at least one vial, test tube, flask, bottle, syringe or other container means, into which a component may be placed, and preferably, suitably aliquoted. Where there is more than one component in the kit (labeling reagent and label may be packaged together), the kit also will generally contain a second, third or other additional container into which the additional components may be separately placed. However, various combinations of components may be comprised in a vial. The kits of the present invention also will typically include a means for containing the nucleic acids, and any other reagent containers in close confinement for commercial sale. Such containers may include injection or blow molded plastic containers into which the desired vials are retained.

When the components of the kit are provided in one and/or more liquid solutions, the liquid solution is an aqueous solution, with a sterile aqueous solution being particularly preferred.

However, the components of the kit may be provided as dried powder(s). When reagents and/or components are provided as a dry powder, the powder can be reconstituted by the addition of a suitable solvent. It is envisioned that the solvent may also be provided in another container means. In some embodiments, labeling dyes are provided as a dried power. It is contemplated that 10, 20, 30, 40, 50, 60, 70, 80, 90, 100, 120, 120, 130, 140, 150, 160, 170, 180, 190, 200, 300, 400, 500, 600, 700, 800, 900, 1000 μg or at least or at most those amounts of dried dye are provided in kits of the invention. The dye may then be resuspended in any suitable solvent, such as DMSO.

Such kits may also include components that facilitate isolation of the labeled miRNA. It may also include components that preserve or maintain the miRNA or that protect against its degradation. Such components may be RNAse-free or protect against RNAses. Such kits generally will comprise, in suitable means, distinct containers for each individual reagent or solution.

A kit will also include instructions for employing the kit components as well the use of any other reagent not included in the kit. Instructions may include variations that can be implemented.

Kits of the invention may also include one or more of the following: Control RNA; nuclease-free water; RNase-free containers, such as 1.5 ml tubes; RNase-free elution tubes; PEG or dextran; ethanol; acetic acid; sodium acetate; ammonium acetate; guanidinium; detergent; nucleic acid size marker; RNase-free tube tips; and RNase or DNase inhibitors.

It is contemplated that such reagents are embodiments of kits of the invention. Such kits, however, are not limited to the particular items identified above and may include any reagent used for the manipulation or characterization of miRNA.

### VII. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Gene Expression Analysis Following Transfection with HSA-miR-200C

miRNAs are believed to regulate gene expression by binding to target mRNA transcripts and (1) initiating transcript degradation or (2) altering protein translation from the transcript. Translational regulation leading to an up or down change in protein expression may lead to changes in activity and expression of downstream gene products and genes that are in turn regulated by those proteins. These numerous regulatory effects may be revealed as changes in the global mRNA expression profile. Microarray gene expression analyses were performed to identify genes that are mis-regulated by hsa-miR-200 expression.

Synthetic Pre-miR-200c (Ambion) or two negative control miRNAs (pre-miR-NC1, Ambion cat. no. AM17110 and pre-miR-NC2, Ambion, cat. no. AM17111) were reverse transfected into quadruplicate samples of A549 cells for each of three time points. Cells were transfected using siPORT NeoFX (Ambion) according to the manufacturer's recommendations using the following parameters: 200,000 cells per well in a 6 well plate, 5.0 μl of NeoFX, 30 nM final concentration of miRNA in 2.5 ml. Cells were harvested at 4 h, 24 h, and 72 h post transfection. Total RNA was extracted using RNAqueous-4PCR (Ambion) according to the manufacturer's recommended protocol.

mRNA array analyses were performed by Asuragen Services (Austin, Tex.), according to the company's standard operating procedures. Using the MessageAMp™ II-96 aRNA Amplification Kit (Ambion, cat #1819) 2 μg of total RNA were used for target preparation and labeling with biotin. cRNA yields were quantified using an Agilent Bioanalyzer 2100 capillary electrophoresis protocol. Labeled target was hybridized to Affymetrix mRNA arrays (Human HG-U133A 2.0 arrays) using the manufacturer's recommendations and the following parameters. Hybridizations were carried out at 45° C. for 16 hr in an Affymetrix Model 640 hybridization oven. Arrays were washed and stained on an Affymetrix FS450 Fluidics station, running the wash script Midi_euk2v3_{—}450. The arrays were scanned on a Affymetrix GeneChip Scanner 3000. Summaries of the image signal data, group mean values, p-values with significance flags, log ratios and gene annotations for every gene on the array were generated using the Affymetrix Statistical Algorithm MAS 5.0 (GCOS v1.3). Data were reported in a file (cabinet) containing the Affymetrix data and result files and in files (.cel) containing the primary image and processed cell intensities of the arrays. Data were normalized for the effect observed by the average of two negative control microRNA sequences and then were averaged together for presentation. A list of genes whose expression levels varied by at least 0.7 log₂ from the average negative control was assembled. Results of the microarray gene expression analysis are shown in Table 1.

Manipulation of the expression levels of the genes listed in Table 1 represents a potentially useful therapy for cancer and other diseases in which increased or reduced expression of hsa-miR-200c has a role in the disease.

### Example 2

### Cellular Pathways Affected by Hsa-miR-200C

The mis-regulation of gene expression by hsa-miR-200c (Table 1) affects many cellular pathways that represent potential therapeutic targets for the control of cancer and other diseases and disorders. The inventors determined the identity and nature of the cellular genetic pathways affected by the regulatory cascade induced by hsa-miR-200c expression. Cellular pathway analyses were performed using Ingenuity Pathways Analysis (Version 4.0, Ingenuity® Systems, Redwood City, Calif.). Alteration of a given pathway was determined by Fisher's Exact test (Fisher, 1922). The most significantly affected pathways following over-expression of hsa-miR-200c in A549 cells are shown in Table 2.

These data demonstrate that hsa-miR-200c directly or indirectly affects the expression of numerous cancer-, cellular proliferation-, cellular development-, cell signaling-, and cell growth-related genes and thus primarily affects functional pathways related to cancer, cellular growth, cell development, and cell proliferation. Those cellular processes all have integral roles in the development and progression of various cancers. Manipulation of the expression levels of genes in the cellular pathways shown in Table 2 represents a potentially useful therapy for cancer and other diseases in which increased or reduced expression of hsa-miR-200c has a role in the disease.

### Example 3

### Predicted Gene Targets of Hsa-miR-200C

Gene targets for binding of and regulation by hsa-miR-200c were predicted using the proprietary algorithm miRNA Target™ (Asuragen), which is an implementation of the method proposed by Krek et al. (2005). Predicted target genes are shown in Table 3.

The predicted gene targets that exhibited altered mRNA expression levels in human cancer cells, following transfection with pre-miR hsa-miR-200c, are shown in Table 4.

The predicted gene targets of hsa-miR-200c whose mRNA expression levels are affected by hsa-miR-200c represent particularly useful candidates for cancer therapy and therapy of other diseases through manipulation of their expression levels.

### Example 4

### Cancer Related Gene Expression Altered by Hsa-miR-200C

Cell proliferation and survival pathways are commonly altered in tumors (Hanahan and Weinberg, 2000). The inventors have shown that hsa-miR-200c directly or indirectly regulates the transcripts of proteins that are critical in the regulation of these pathways. Many of these targets have inherent oncogenic or tumor suppressor activity. Hsa-miR-200c targets that have prognostic and/or therapeutic value for the treatment of various malignancies are shown in Table 5.

Hsa-miR-200c targets of particular interest are genes and their products that function in the regulation of intracellular signal transduction. When deregulated, many of these proteins contribute to the malignant phenotype in vitro and in vivo. Hsa-miR-200c controls the expression of secretory growth factors and transmembrane growth factor receptors. Examples of secreted proteins regulated by hsa-miR-200c are amphiregulin (AREG), fibroblast growth factor-binding protein 1 (FGFBP1), connective tissue growth factor (CTGF), insulin growth factor-binding protein 1 (IGFBP1) and the inflammatory chemokine IL-8 (Firth and Baxter, 2002; Sparmann and Bar-Sagi, 2004). Amphiregulin functions as a ligand to epidermal growth factor receptor (EGFR) and activates EGFR dependent signaling (Hynes and Lane, 2005). Amphiregulin is frequently expressed in ovarian, gastric and pancreatic carcinoma as well as hepatocellular carcinoma tissues and cell lines (Kitadai et al., 1993; Ebert et al., 1994; D'Antonio et al., 2002; Castillo et al., 2006). Amphiregulin acts as a mitogenic and anti-apoptotic growth factor in hepatocarcinoma cells and contributes to the transformed phenotype of liver cancer cells. Inhibition of amphiregulin function by small interfering RNA (siRNA) or neutralizing antibodies diminishes the amphiregulin-mediated autocrine loop and oncogenic properties of hepatocarcinoma cells (Castillo et al., 2006). Amphiregulin expression also progressively increases from benign to malignant stages of prostate cancer and is indicative for poor response to treatment with the FDA-approved drug Iressa (gefitinib) in patients with non-small cell lung cancer (NSCLC) (Bostwick et al., 2004; Ishikawa et al., 2005). FGFBP1 is a secretory protein stored in an inactive form on heparin sulfate proteoglycans in the extracellular matrix (Tassi et al., 2001; Abuharbeid et al., 2006). It has high affinity for FGF-1 and FGF-2 and functions as chaperone to mobilize locally stored FGF. Thus, FGFBP1 is a positive regulator of FGFs enhancing FGF signaling and angiogenesis (Tassi et al., 2001). FGFBP1 expression is highly tissue specific and absent in most normal adult tissues. Yet, FGFBP1 is overexpressed in various types of cancer, including cancers of the breast, colon and prostate (Abuharbeid et al., 2006). High FGFBP1 expression is associated with early stages of tumor development, contributing to tumor angiogenesis. CTGF (also referred to as insulin-like growth factor binding protein 8; IGFBP8) was originally described as a mitogen produced by umbilical vein endothelial cells (Bradham et al., 1991). Similar to FGFBP1, it functions as a modulator of growth factor activity and is overexpressed in various tumors (Hishikawa et al., 1999; Shimo et al., 2001; Lin et al., 2005; Yang et al., 2005). CTGF is induced by hypoxia and enhances angiogenesis as well as the growth of tumor xenografts (Shimo et al., 2001; Yang et al., 2005). However, a coherent role for CTGF in cancer remains elusive and may depend on the cellular context (Hishikawa et al., 1999; Lin et al., 2005). Transmembrane receptors targeted by hsa-miR-200c include retinoic acid receptor responder 1 (RARRES1) and fibroblast growth factor receptor 4 (FGFR4). FGFR-4 is commonly overexpressed in multiple cancer types and appears to have angiogenic activity (Chandler et al., 1999). In contrast, RARRES1 is a putative tumor suppressor that is lost or shows decreased expression levels in several types of cancer (Wu et al., 2006 and references therein).

Hsa-miR-200c also governs the expression of Fas and MCL1, both of which are functionally linked to the apoptotic pathway. MCL1 is a member of the anti-apoptotic BCL-2 (B cell lymphoma 2) gene family that give rise to two alternatively spliced gene products with opposing functions (Boise et al., 1993; Bae et al., 2000). High levels of MCL1 are correlated with poor prognosis of patients with ovarian carcinoma and is indicative for leukemic relapse (Kaufmann et al., 1998; Shigemasa et al., 2002). RNA interference against MCL1 induces a therapeutic response in gastric and hepatocellular carcinoma cells (Schulze-Bergkamen et al., 2006; Zangemeister-Wittke and Huwiler, 2006). Fas, also known as CD95 or APO-1, is a transmembrane cell surface receptor that functions in the transduction of apoptotic signals in response to its ligand FasL (Houston and O'Connell, 2004). Reduced Fas expression is a common mechanism of cells to decrease the sensitivity to FasL-mediated cell death. Similarly, many different cancer types show lost or decreased Fas expression levels (Table 5). In colorectal carcinoma, Fas expression is progressively reduced in the transformation of normal epithelium to benign neoplasm, adenocarcinomas and metastases (Moller et al., 1994). Thus, despite expression of FasL, tumor cells may escape the FasL induced apoptotic signal. Transient transfection of hsa-miR-200c results in an increase of Fas transcripts and therefore may restore sensitivity to FasL in cancer cells.

Another class of genes regulated by hsa-miR-200c encodes proteins that function in the progression of the cell cycle. Among these are retinoblastoma-like 1 protein (RBL1) as well as cyclin G1 (CCNG1). RBL1, also known as p107, is a member of the retinoblastoma tumor suppressor protein family that includes the pocket proteins p107, p130 and pRb. Similar to the pRb prototype, RBL1 interacts with the E2F family of transcription factors and blocks cell cycle progression and DNA replication (Sherr and McCormick, 2002). A subset of cancers show deregulated expression of RBL1 (Takimoto et al., 1998; Claudio et al., 2002; Wu et al., 2002; Ito et al., 2003). Cyclins are co-factors of cyclin-dependent kinases (CDKs) necessary in the progression of the cell cycle. In contrast to most cyclins, however, cyclin G1 has growth inhibitory activity (Zhao et al., 2003).

Further growth-related genes regulated by hsa-miR-200c include thioredoxin (TXN), a 12-kDa thiol reductase targeting various proteins and multiple pathways. Thioredoxin modulates the activity of transcription factors, induces the expression of angiogenic Hif-1α (hypoxia induced factor 1α) as well as VEGF (vascular endothelial growth factor) and can act as a proliferative and anti-apoptotic agent (Marks, 2006). In accord, carcinomas of the lung, pancreas, cervix and liver show increased levels of thioredoxin. Thioredoxin expression is also correlated with aggressive tumor growth, poor prognosis and chemoresistance (Marks, 2006).

In summary, hsa-miR-200c governs the activity of proteins that are critical regulators of cell proliferation and survival. These targets are frequently deregulated in human cancer. Based on this review of the genes and related pathways that are regulated by miR-200c, introduction of hsa-miR-200c or an anti-hsa-miR-200c into a variety of cancer cell types would likely result in a therapeutic response.

### REFERENCES

The following references, to the extent that they provide exemplary procedural or other details supplementary to those set forth herein, are specifically incorporated herein by reference.
U.S. Pat. No. 4,337,063
U.S. Pat. No. 4,404,289
U.S. Pat. No. 4,405,711
U.S. Pat. No. 4,659,774
U.S. Pat. No. 4,682,195
U.S. Pat. No. 4,683,202
U.S. Pat. No. 4,704,362
U.S. Pat. No. 4,816,571
U.S. Pat. No. 4,870,287
U.S. Pat. No. 4,959,463
U.S. Pat. No. 5,141,813
U.S. Pat. No. 5,143,854
U.S. Pat. No. 5,202,231
U.S. Pat. No. 5,214,136
U.S. Pat. No. 5,221,619
U.S. Pat. No. 5,223,618
U.S. Pat. No. 5,242,974
U.S. Pat. No. 5,264,566
U.S. Pat. No. 5,264,566
U.S. Pat. No. 5,268,486
U.S. Pat. No. 5,288,644
U.S. Pat. No. 5,324,633
U.S. Pat. No. 5,378,825
U.S. Pat. No. 5,384,261
U.S. Pat. No. 5,399,363
U.S. Pat. No. 5,405,783
U.S. Pat. No. 5,412,087
U.S. Pat. No. 5,424,186
U.S. Pat. No. 5,428,148
U.S. Pat. No. 5,429,807
U.S. Pat. No. 5,432,049
U.S. Pat. No. 5,436,327
U.S. Pat. No. 5,445,934
U.S. Pat. No. 5,446,137
U.S. Pat. No. 5,466,468
U.S. Pat. No. 5,466,786
U.S. Pat. No. 5,468,613
U.S. Pat. No. 5,470,710
U.S. Pat. No. 5,470,967
U.S. Pat. No. 5,472,672
U.S. Pat. No. 5,480,980
U.S. Pat. No. 5,492,806
U.S. Pat. No. 5,503,980
U.S. Pat. No. 5,510,270
U.S. Pat. No. 5,525,464
U.S. Pat. No. 5,525,464
U.S. Pat. No. 5,527,681
U.S. Pat. No. 5,529,756
U.S. Pat. No. 5,532,128
U.S. Pat. No. 5,543,158
U.S. Pat. No. 5,545,531
U.S. Pat. No. 5,547,839
U.S. Pat. No. 5,554,501
U.S. Pat. No. 5,554,744
U.S. Pat. No. 5,556,752
U.S. Pat. No. 5,561,071
U.S. Pat. No. 5,571,639
U.S. Pat. No. 5,574,146
U.S. Pat. No. 5,580,726
U.S. Pat. No. 5,580,732
U.S. Pat. No. 5,583,013
U.S. Pat. No. 5,593,839
U.S. Pat. No. 5,599,672
U.S. Pat. No. 5,599,695
U.S. Pat. No. 5,602,240
U.S. Pat. No. 5,602,244
U.S. Pat. No. 5,610,289
U.S. Pat. No. 5,610,287
U.S. Pat. No. 5,614,617
U.S. Pat. No. 5,623,070
U.S. Pat. No. 5,624,711
U.S. Pat. No. 5,631,134
U.S. Pat. No. 5,637,683
U.S. Pat. No. 5,639,603
U.S. Pat. No. 5,641,515
U.S. Pat. No. 5,645,897
U.S. Pat. No. 5,652,099
U.S. Pat. No. 5,654,413
U.S. Pat. No. 5,658,734
U.S. Pat. No. 5,661,028
U.S. Pat. No. 5,665,547
U.S. Pat. No. 5,667,972
U.S. Pat. No. 5,670,663
U.S. Pat. No. 5,672,697
U.S. Pat. No. 5,677,195
U.S. Pat. No. 5,681,947
U.S. Pat. No. 5,695,940
U.S. Pat. No. 5,700,637
U.S. Pat. No. 5,700,922
U.S. Pat. No. 5,705,629
U.S. Pat. No. 5,708,153
U.S. Pat. No. 5,708,154
U.S. Pat. No. 5,714,606
U.S. Pat. No. 5,728,525
U.S. Pat. No. 5,739,169
U.S. Pat. No. 5,744,305
U.S. Pat. No. 5,760,395
U.S. Pat. No. 5,763,167
U.S. Pat. No. 5,770,358
U.S. Pat. No. 5,777,092
U.S. Pat. No. 5,789,162
U.S. Pat. No. 5,792,847
U.S. Pat. No. 5,800,992
U.S. Pat. No. 5,801,005
U.S. Pat. No. 5,807,522
U.S. Pat. No. 5,824,311
U.S. Pat. No. 5,830,645
U.S. Pat. No. 5,830,880
U.S. Pat. No. 5,837,196
U.S. Pat. No. 5,846,225
U.S. Pat. No. 5,846,945
U.S. Pat. No. 5,847,219
U.S. Pat. No. 5,856,174
U.S. Pat. No. 5,858,988
U.S. Pat. No. 5,859,221
U.S. Pat. No. 5,871,928
U.S. Pat. No. 5,872,232
U.S. Pat. No. 5,876,932
U.S. Pat. No. 5,886,165
U.S. Pat. No. 5,919,626
U.S. Pat. No. 5,922,591
U.S. Pat. No. 6,004,755
U.S. Pat. No. 6,040,193
U.S. Pat. No. 6,087,102
U.S. Pat. No. 6,251,666
U.S. Pat. No. 6,368,799
U.S. Pat. No. 6,383,749
U.S. Pat. No. 6,617,112
U.S. Pat. No. 6,638,717
U.S. Pat. No. 6,720,138
U.S. Pat. No. 6,723,509
U.S. application Ser. No. 09/545,207
U.S. application Ser. No. 10/667,126
U.S. application Ser. No. 11/141,707
U.S. application Ser. No. 11/141,707
U.S. application Ser. No. 11/141,707
U.S. application Ser. No. 11/273,640
U.S. application Ser. No. 11/273,640,
U.S. application Ser. No. 11/273,640,
U.S. application Ser. No. 11/349,727
U.S. Prov. Appln. Ser. 60/575,743
U.S. Prov. Appln. Ser. 60/649,584
U.S. Prov. Appln. Ser. 60/650,807
Abuharbeid et al., Int. J. Biochem. Cell Biol., 38(9):1463-1468, 2006.
Akiba et al., Int. J. Oncol., 18(2):257-264, 2001.
Arap et al., Cancer Res., 55(6):1351-1354, 1995.
Austin-Ward and Villaseca, Revista Medica de Chile, 126(7):838-845, 1998.
Bae et al., J. Biol. Chem., 275(33):25255-25261, 2000.
Bagga et al., Cell, 122(4):553-563, 2005.
Bartsch and Tschesche, FEBS Lett., 357(3):255-259, 1995.
Boise et al., Cell, 74(4):597-608, 1993.
Bostwick et al., Prostate, 58(2):164-168, 2004.
Bradham et al., J. Cell Biol., 114(6):1285-1294, 1991.
Bukowski et al., Clinical Cancer Res., 4(10):2337-2347, 1998.
Caldas et al., Cancer Res., 54:3568-3573, 1994.
Calin and Croce, Nat. Rev. Cancer, 6(11):857-866, 2006.
Carrington and Ambros, Science, 301(5631):336-338, 2003.
Castillo et al., Cancer Res., 66(12):6129-6138, 2006.
Chandler et al., Int. J. Cancer, 81(3):451-458, 1999.
Cheng et al., Cancer Res., 54(21):5547-5551, 1994.
Cho-Vega et al., Hum. Pathol., 35(9):1095-1100, 2004.
Christodoulides et al., Microbiology, 144(Pt 11):3027-3037, 1998.
Claudio et al., Clin. Cancer Res., 8(6):1808-1815, 2002.
Croci et al., Cancer Res., 64(5):1730-1736, 2004.
Cummins et al., In: IRT: Nucleosides and nucleosides, La Jolla Calif., 72, 1996.
D'Antonio et al., Int. J. Oncol., 21(5):941-948, 2002.
Davidson et al., J. Immunother., 21(5):389-398, 1998.
Denli et al., Trends Biochem. Sci., 28:196, 2003.
Didenko, Biotechniques, 31(5):1106-1116, 1118, 1120-1121, 2001.
Dillman, Cancer Biother. Radiopharm., 14(1):5-10, 1999.
Dong et al., Mol. Endocrinol., 20(10):2315-2325, 2006.
Ebert et al., Cancer Res., 54(15):3959-3962, 1994.
Emptage et al., Neuron, 29(1):197-208, 2001.
EP 266,032
EP 373 203
EP 785 280
EP 799 897
Esquela-Kerscher and Slack, Nat. Rev. Cancer, 6(4):259-269, 2006.
Ezzat et al., Clin. Cancer Res., 11(3):1336-1341, 2005.
Fernandez et al., Clin. Cancer Res., 11(15):5390-5395, 2005.
Firth and Baxter, Endocr. Rev., 23(6):824-854, 2002.
Fisher, J. Royal Statistical. Soc. 85(1):87-94, 1922.
Fleischer et al., Int. J. Oncol., 28(1):25-32, 2006.
Fodor et al., Biochemistry, 30(33):8102-8108, 1991.
Froehler et al., Nucleic Acids Res., 14(13):5399-5407, 1986.
Furutani et al., Cancer Lett., 122(1-2):209-214, 1998.
Gratas et al., Cancer Res., 58(10):2057-2062, 1998.
Griffey et al., J. Mass Spectrom, 32(3):305-13, 1997.
Hanibuchi et al., Int. J. Cancer, 78(4):480-485, 1998.
Hellstrand et al., Acta Oncologica, 37(4):347-353, 1998.
Hishikawa et al., J. Biol. Chem., 274(52):37461-37466, 1999.
Hooi et al., Oncogene, 25(28):3924-3933, 2006.
Houston and O'Connell, Curr. Opin. Pharmacol., 4(4):321-326, 2004.
Hui and Hashimoto, Infection Immun., 66(11):5329-5336, 1998.
Hussussian et al., Nat. Genet., 8(1):15-21, 1994.
Hynes and Lane, Nat. Rev. Cancer, 5(5):341-354, 2005.
Ishikawa et al., Cancer Res., 65(20):9176-9184, 2005.
Itakura and Riggs, Science, 209:1401-1405, 1980.
Ito et al., Anticancer Res., 23(5A):3819-3824, 2003.
Jaakkola et al., Int. J. Cancer, 54(3):378-382, 1993.
Ju et al., Gene Ther., 7(19):1672-1679, 2000.
Kamb et al., Science, 2674:436-440, 1994.
Kaufmann et al., Blood, 91(3):991-1000, 1998.
Kitada et al., Blood, 91(9):3379-3389, 1998.
Kitadai et al., Jpn. J. Cancer Res., 84(8):879-884, 1993.
Klostermeier and Millar, Biopolymers, 61(3):159-79, 2001-2002.
Koliopanos et al., World J. Surg., 26(4):420-427, 2002.
Krajewska et al., Am. J. Pathol., 148(5):1567-1576, 1996.
Krek et al., Nature Genet. 37:495-500, 2005.
Lagos-Quintana et al., Science, 294(5543):853-858, 2001.
Lau et al., Science, 294(5543):858-862, 2001.
Lee and Ambros, Science, 294(5543):862-864, 2001.
Lee et al., Int. J. Cancer, 118(10):2490-2497, 2006.
Lee et al., Oncogene, 23(39):6672-6676, 2004.
Lin et al., Gastroenterology, 128(1):9-23, 2005.
Liu and Matsuura, Cell Cycle, 4(1):63-66, 2005.
Liu et al., Cancer Res., 66(2):653-658, 2006.
Mahtouk et al., Oncogene, 24(21):3512-3524, 2005.
Marks, Semin. Cancer Biol., 16(6):436-443, 2006.
Marsit et al., Int. J. Cancer, 1119(8):1761-1766, 2006.
Marsters et al., Recent Prog. Horm. Res., 54:225-234, 1999.
Martinez-Lorenzo et al., Int. J. Cancer, 75(3):473-481, 1998.
Meng et al., Gastroenterology, 130:2113-2129, 2006.
Moller et al., Int J Cancer, 57(3):371-377, 1994.
Mori et al., Cancer Res., 54(13):3396-3397, 1994.
Nobri et al., Nature (London), 368:753-756, 1995.
Okamoto et al., Proc. Natl. Acad. Sci. USA, 91(23):11045-11049, 1994.
Olsen et al., Dev. Biol., 216:671, 1999.
Orlow et al., Cancer Res, 54(11):2848-2851, 1994.
Pan et al., Neurol. Res., 24(7):677-683, 2002.
PCT Appln. WO 0138580
PCT Appln. WO 0168255
PCT Appln. WO 03020898
PCT Appln. WO 03022421
PCT Appln. WO 03023058
PCT Appln. WO 03029485
PCT Appln. WO 03040410
PCT Appln. WO 03053586
PCT Appln. WO 03066906
PCT Appln. WO 03067217
PCT Appln. WO 03076928
PCT Appln. WO 03087297
PCT Appln. WO 03091426
PCT Appln. WO 03093810
PCT Appln. WO 03100012
PCT Appln. WO 03100448A1
PCT Appln. WO 04020085
PCT Appln. WO 04027093
PCT Appln. WO 09923256
PCT Appln. WO 09936760
PCT Appln. WO 93/17126
PCT Appln. WO 95/11995
PCT Appln. WO 95/21265
PCT Appln. WO 95/21944
PCT Appln. WO 95/21944
PCT Appln. WO 95/35505
PCT Appln. WO 96/31622
PCT Appln. WO 97/10365
PCT Appln. WO 97/27317
PCT Appln. WO 9743450
PCT Appln. WO 99/35505
Petit et al., Genomics, 57(3):438-441, 1999.
Pietras et al., Oncogene, 17(17):2235-2249, 1998.
Pruitt et al., Nucleic Acids Res., 33(1):D501-D504, 2005.
Qin et al., Proc. Natl. Acad. Sci. USA, 95(24):14411-14416, 1998.
Reimer et al., J. Biol. Chem., 274(16):11022-11029, 1999.
Remington's Pharmaceutical Sciences” 15th Edition, pages 1035-1038 and 1570-1580, 1990.
Rust et al., J. Clin. Pathol., 58(5):520-524, 2005.
Sambrook and Russell, Molecular Cloning: A Laboratory Manual 3^{rd} Ed., Cold Spring Harbor Laboratory Press, 2001.
Sambrook et al., In: DNA microarrays: a molecular cloning manual, Cold Spring Harbor Laboratory Press, Cold Spring Harbor, N.Y., 2003.
Sambrook et al., In: Molecular cloning: a laboratory manual, 2^{nd} Ed., Cold Spring Harbor Laboratory Press, Cold Spring Harbor, N.Y., 1989.
Sano et al., Histopathology, 46(5):532-539, 2005.
Schulze-Bergkamen et al., BMC Cancer, 6:232, 2006.
Seggerson et al., Dev. Biol., 243:215, 2002.
Serrano et al., Nature, 366:704-707, 1993.
Serrano et al., Science, 267(5195):249-252, 1995.
Shah et al., Oncogene, 21(54):8251-8261, 2002.
Sherr and McCormick, Cancer Cell, 2(2):103-112, 2002.
Shigemasa et al., Jpn. J. Cancer Res, 93(5):542-550, 2002.
Shimo et al., Cancer Lett., 174(1):57-64, 2001.
Shinoura et al., Cancer Gene Ther., 7(2):224-232, 2000.
Sieghart et al., J. Hepatol., 44(1):151-157, 2006.
Skotzko et al., Cancer Res., 55(23):5493-5498, 1995.
Solic and Davies, Exp. Cell Res., 234(2):465-476, 1997.
Sparmann and Bar-Sagi, Cancer Cell, 6(5):447-458, 2004.
Takimoto et al., Biochem. Biophys. Res. Commun., 251(1):264-268, 1998.
Tassi et al., Cancer Res., 66(2):1191-1198, 2006.
Tassi et al., J. Biol. Chem., 276(43):40247-40253, 2001.
UK Appln. 8 803 000
UK Patent 1,529,202
Viard-Leveugle et al., J. Pathol., 201(2):268-277, 2003.
Wu et al., Eur. J. Cancer, 38(14):1838-1848, 2002.
Wu et al., Eur. J. Cancer, 42(4):557-565, 2006.
Wuilleme-Toumi et al., Leukemia, 19(7):1248-1252, 2005.
Xi et al., Clin. Cancer Res., 12(8):2484-2491, 2006a.
Xi et al., Clin. Chem., 52(3):520-523, 2006b.
Yang et al., Cancer Res., 65(19):8887-8895, 2005.
Zangemeister-Wittke and Huwiler, Cancer Biol. Ther., 5(10):1355-1356, 2006.
Zhang et al., Oncogene, 23(12):2241-2249, 2004.
Zhao et al., Mol. Cancer. Res., 1(3):195-206, 2003.

1. A method of modulating gene expression in a cell comprising administering to the cell an amount of an isolated nucleic acid comprising a miR-200 nucleic acid sequence in an amount sufficient to modulate the expression of one or more genes identified in Table 1, 3, 4, or 5.

2. The method of claim 1, wherein the cell is in a subject having, suspected of having, or at risk of developing a metabolic, an immunologic, an infectious, a cardiovascular, a digestive, an endocrine, an ocular, a genitourinary, a blood, a musculoskeletal, a nervous system, a congenital, a respiratory, a skin, or a cancerous disease or condition.

3. (canceled)

4. The method of claim 2, wherein the cancerous condition is anaplastic large cell lymphoma, breast carcinoma, B-cell lymphoma, cervical carcinoma, chronic lymphoblastic leukemia, colorectal carcinoma, glioma, glioblastoma, gastric carcinoma, hepatocellular carcinoma, leukemia, lung carcinoma, lipoma, multiple myeloma, mesothelioma, non-small cell lung carcinoma, ovarian carcinoma, oesophageal carcinoma, osteosarcoma, pancreatic carcinoma, prostate carcinoma, rhabdomyosarcoma, squamous cell carcinoma of the head and neck, thyroid carcinoma, or testicular tumor wherein the modulation of one or more gene is sufficient for a therapeutic response.

5. The method of claim 1, wherein the expression of a gene is down-regulated.

6. The method of claim 1 wherein the expression of a gene is up-regulated.

7. (canceled)

8. (canceled)

9. The method of claim 1, wherein the cell is a cancer cell.

10. The method of claim 9, wherein the cancer cell is a colorectal, neuronal, glial, lung, liver, brain, breast, bladder, blood, leukemic, colon, endometrial, epithelial, intestinal, mesothelial, stomach, skin, ovarian, fat, bone, cervical, esophageal, pancreatic, prostate, kidney, testicular, or thyroid cell.

11. The method of claim 1, wherein the isolated miR-200 nucleic acid is a recombinant nucleic acid.

12.-16. (canceled)

17. The method of claim 1, wherein the miR-200 nucleic acid is a synthetic nucleic acid.

18. The method of claim 17, wherein the nucleic acid is administered at a dose of 0.01 mg/kg of body weight to 10 mg/kg of body weight.

19. The method of claim 1, wherein the miR-200 is a hsa-miR-200.

20. The method of claim 1, wherein the miR-200 is miR-200a, miR-200b, or miR-200c.

21. The method of claim 1, wherein the nucleic acid is administered enterally.

22. (canceled)

23. (canceled)

24. The method of claim 1, wherein the nucleic acid is comprised in a pharmaceutical formulation.

25. The method of claim 24, wherein the pharmaceutical formulation is a lipid composition.

26. The method of claim 24 wherein the pharmaceutical formulation is a nanoparticle composition.

27. The method of claim 24 wherein the pharmaceutical formulation consists of biocompatible and biodegradable molecules.

28.-43. (canceled)

44. A method of treating a patient diagnosed with or suspected of having or suspected of developing a pathological condition or disease related to a gene modulated by a miRNA comprising the steps of:
(a) administering to the patient an amount of an isolated nucleic acid comprising a miR-200 nucleic acid sequence in an amount sufficient to modulate a cellular pathway or a physiologic pathway; and
(b) administering a second therapy, wherein the modulation of the cellular pathway or physiologic pathway sensitizes the patient to the second therapy.

45. The method of claim 44, wherein one or more cellular pathway or physiologic pathway includes one or more genes identified in Table 1, 3, 4, or 5.

46.-50. (canceled)

51. A method of assessing miR-200 status in a sample comprising the steps of:
(a) assessing expression of one or more genes from Table 1, 3, 4, or 5 in a sample; and
(b) determining miR-200 status based on level of miR-200 expression in the sample.
